SEQUENCE LISTING (44 sequences)

SEQ ID NO: 1 - protein, 120 aa, synthetic construct
QVQLVQSGAEVKKPGASVKVSCKASGYTFTSYVMHWVRQAPGQRLEWMGYINSYSDGTKYNEKFKGRVTITSDTSASTAYMELSSLRSEDTAVYYCARWGGLGNGAMDYWGQGTLVTVSS

SEQ ID NO: 2 - protein, 120 aa, synthetic construct
QVQLVQSGAEVKKPGASVKVSCKASGYTFTSYVMHWVRQAPGQRLEWMGYINSYSDGTKYNEKFKGRATITRDTSASTAYMELSSLRSEDTAVYYCARWGGLGNGAMDYWGQGTLVTVSS

SEQ ID NO: 3 - protein, 120 aa, synthetic construct
QVQLVQSGAEVKKPGASVKVSCKASGYTFTSYVMHWVRQAPGQRLEWMGYINSYSDGTKYNEKFKGRVTITRDTSASTAYMELSSLRSEDTAVYYCARWGGLGNGAMDYWGQGTLVTVSS

SEQ ID NO: 4 - protein, 106 aa, synthetic construct
EIVLTQSPATLSLSPGERATLSCSVSSSVNYMHWYQQKPGQAPRRLIYDTSKLASGIPARFSGSGSGTDYTLTISSLEPEDFAVYYCQQWTSNPLTFGQGTKLEIK

SEQ ID NO: 5 - DNA, 1413 bp, synthetic construct
  source 1..1413: mol_type = other DNA
atgggctggtcatgcattattctgtttctggtcgcaactgctacaggcgtgcatagtgaggtgcagctgcagcagagcggacctgagctggtcaagcccggcgcttctgtgaaaatgagctgtaaagccagcggctacaccttcacctcttatgtgatgcactgggtgcggcagtctcctggacaaggcctggaaggcatcggctacatcaacagctacagcgacggcacaaagtacaacgagaagttcaagggcaaggctacactgacctccgataagtccagctctacagcctacatggaactgagcggcctgaccagcgaggacagcgccgtgtactactgcgccagatggggaggactgggcaatggcgccatggactattggggccagggcaccagcgtgaccgtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 6 - DNA, 702 bp, synthetic construct
  source 1..702: mol_type = other DNA
atgggctggtcatgtattattctgtttctggtcgcaactgctacaggggtccatagtcagatcgtgctgacccagagccctgccatcatgagcgcctcccctggcgagaaggtgaccatgacctgtagcgtgtccagctctgtgaactacatgcactggtatcagcagaagagcggcacatctccaaagcggtggatctacgatacctctaaactggccagcggcgtccccgctagattcagtgcctctggaagcggaaccagctacagcctgacaatcagcagcatggaagccgaggacgccgctacatactactgccagcaatggacaagcaaccctctgaccttcggcgccggcaccaagctggaactgaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 7 - DNA, 1413 bp, synthetic construct
  source 1..1413: mol_type = other DNA
atgggctggtcatgcattattctgtttctggtcgcaactgctacaggcgtgcatagtcaggtgcagctggtgcagagcggcgctgaggtgaagaagcccggcgccagcgtgaaagtgtcttgtaaagcctctggctacaccttcaccagctacgtgatgcactgggtcagacaggcccctggacagcggctggaatggatgggctatatcaacagctacagcgacggcaccaagtacaacgagaagttcaagggcagagtgacaatcacccgggacaccagcgccagcacagcctacatggaactgtctagcctgagaagcgaggataccgccgtgtactactgcgccagatggggcggcctgggcaatggcgctatggactattggggacaaggaaccctggttacagtgtcctccgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 8 - DNA, 702 bp, synthetic construct
  source 1..702: mol_type = other DNA
atgggctggtcatgtattattctgtttctggtcgcaactgctacaggggtccatagtgagatcgtgctgacccagagcccagccacactgagcctgagccctggcgagcgggccaccctctcctgcagcgtgtccagctccgtcaactacatgcactggtatcagcagaaacctggccaggcccctagactgctgatctacgacaccagcaagctggcctctggaatccctgctagattcagcggatctggcagcggcaccgattttaccctgacaatctctagcctggaacccgaggacttcgccgtgtactactgtcagcaatggaccagcaaccccctgacattcggccagggcaccaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 9 - protein, 12 aa, synthetic construct
IMGTGYTFTSYV

SEQ ID NO: 10 - protein, 8 aa, synthetic construct
INSYSDGT

SEQ ID NO: 11 - protein, 13 aa, synthetic construct
ARWGGLGNGAMDY

SEQ ID NO: 12 - protein, 5 aa, synthetic construct
SSVNY

SEQ ID NO: 13 - skipped

SEQ ID NO: 14 - protein, 9 aa, synthetic construct
QQWTSNPLT

SEQ ID NO: 15 - protein, 9 aa, synthetic construct
YTFTSYVMH

SEQ ID NO: 16 - protein, 14 aa, synthetic construct
GIGYINSYSDGTKY

SEQ ID NO: 17 - protein, 12 aa, synthetic construct
RWGGLGNGAMDY

SEQ ID NO: 18 - protein, 7 aa, synthetic construct
SSVNYMH

SEQ ID NO: 19 - protein, 11 aa, synthetic construct
RWIYDTSKLAS

SEQ ID NO: 20 - protein, 8 aa, synthetic construct
QQWTSNPL

SEQ ID NO: 21 - protein, 7 aa, synthetic construct
GYTFTSY

SEQ ID NO: 22 - protein, 6 aa, synthetic construct
NSYSDG

SEQ ID NO: 23 - protein, 10 aa, synthetic construct
WGGLGNGAMD

SEQ ID NO: 24 - protein, 10 aa, synthetic construct
SVSSSVNYMH

SEQ ID NO: 25 - protein, 7 aa, synthetic construct
DTSKLAS

SEQ ID NO: 26 - protein, 9 aa, synthetic construct
QQWTSNPLT

SEQ ID NO: 27 - DNA, 1413 bp, synthetic construct
  source 1..1413: mol_type = other DNA
atgggctggtcatgcattattctgtttctggtcgcaactgctacaggcgtgcatagtcaagttcagctggtgcagagcggcgccgaggtgaaaaagcccggcgctagcgtgaaagtgtcctgcaaggccagcggatacaccttcacctcttatgtgatgcactgggtgcggcaggcccctggccagcggctggaatggatgggctacatcaacagctacagcgacggcaccaagtacaacgagaagttcaagggcagagtgaccatcacctccgatacaagcgcctctaccgcctacatggaactgagcagcctgagaagcgaggacacagccgtgtactactgtgccagatggggaggcctgggaaatggcgctatggactactggggccagggcacactggtcaccgtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 28 - DNA, 1413 bp, synthetic construct
  source 1..1413: mol_type = other DNA
atgggctggtcatgcattattctgtttctggtcgcaactgctacaggcgtgcatagtcaagtgcagctggtccagagcggcgccgaggtgaaaaagcccggcgcttctgttaaagtgtcctgcaaggcctctggatatacattcacctcctacgtgatgcactgggtgcggcaggcccctggccagcggctggaatggatgggctacatcaacagctacagcgacggcacaaagtacaacgagaagttcaagggcagagccacaatcaccagagataccagcgccagcaccgcctacatggaactgagcagcctgagaagcgaggacaccgccgtgtactactgtgccagatggggcggactgggcaatggcgctatggactactggggacagggcaccctggtgaccgtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 29 - DNA, 1413 bp, synthetic construct
  source 1..1413: mol_type = other DNA
atgggctggtcatgcattattctgtttctggtcgcaactgctacaggcgtgcatagtcaagtgcagctggttcagagcggcgccgaggtgaaaaagcccggagctagcgtgaaagtgtcctgcaaggcctctggctacaccttcacctcctacgtgatgcactgggtcagacaggcccctggccagcggctggaatggatgggctatatcaacagctacagcgacggcacaaagtacaacgagaagttcaagggcagagtgaccatcaccagagataccagcgcctctacagcctacatggaactgagcagcctgcggagcgaggacaccgccgtgtactactgtgccagatggggaggactgggcaatggcgctatggactactggggccagggcaccctggtgacagtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 30 - DNA, 702 bp, synthetic construct
  source 1..702: mol_type = other DNA
atgggctggtcatgtattattctgtttctggtcgcaactgctacaggggtccatagtgagatcgtgctgacacagagccctgctacactgagcctgagccccggcgagcgggccacactctcctgcagcgtgtccagctctgtcaactacatgcactggtatcagcagaaacctggccaggcccctagaagactgatctacgacaccagcaagctggcctctggaatcccagccagattcagcggatctggcagcggcaccgattacaccctgaccatcagcagcctggaacctgaggacttcgccgtgtactactgtcagcaatggacctccaaccccctgacctttggccagggcaccaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 31 - protein, 469 aa, synthetic construct
MGWSCIILFLVATATGVHSEVQLQQSGPELVKPGASVKMSCKASGYTFTSYVMHWVRQSPGQGLEGIGYINSYSDGTKYNEKFKGKATLTSDKSSSTAYMELSGLTSEDSAVYYCARWGGLGNGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 469 aa, synthetic construct
MGWSCIILFLVATATGVHSQVQLVQSGAEVKKPGASVKVSCKASGYTFTSYVMHWVRQAPGQRLEWMGYINSYSDGTKYNEKFKGRVTITRDTSASTAYMELSSLRSEDTAVYYCARWGGLGNGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 232 aa, synthetic construct
MGWSCIILFLVATATGVHSQIVLTQSPAIMSASPGEKVTMTCSVSSSVNYMHWYQQKSGTSPKRWIYDTSKLASGVPARFSASGSGTSYSLTISSMEAEDAATYYCQQWTSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 232 aa, synthetic construct
MGWSCIILFLVATATGVHSEIVLTQSPATLSLSPGERATLSCSVSSSVNYMHWYQQKPGQAPRLLIYDTSKLASGIPARFSGSGSGTDFTLTISSLEPEDFAVYYCQQWTSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 8 aa, synthetic construct
DYKDDDDK

SEQ ID NO: 36 - protein, 6 aa, synthetic construct
HHHHHH

SEQ ID NO: 37 - protein, 6 aa, synthetic construct
HEHEHE

SEQ ID NO: 38 - protein, 9 aa, synthetic construct
YPYDVPDYA

SEQ ID NO: 39 - protein, 45 aa, synthetic construct
TTTPAPRPPTPAPTIASQPLSLRPEACRPAAGGAVHTRGLDFACD

SEQ ID NO: 40 - protein, 24 aa, synthetic construct
IYIWAPLAGTCGVLLLSLVITLYC

SEQ ID NO: 41 - protein, 42 aa, synthetic construct
KRGRKKLLYIFKQPFMRPVQTTQEEDGCSCRFPEEEEGGCEL

SEQ ID NO: 42 - protein, 242 aa, synthetic construct
EIVLTQSPATLSLSPGERATLSCSVSSSVNYMHWYQQKPGQAPRRLIYDTSKLASGIPARFSGSGSGTDYTLTISSLEPEDFAVYYCQQWTSNPLTFGQGTKLEIKGSGSGSGSGSGSGSGSQVQLVQSGAEVKKPGASVKVSCKASGYTFTSYVMHWVRQAPGQRLEWMGYINSYSDGTKYNEKFKGRVTITRDTSASTAYMELSSLRSEDTAVYYCARWGGLGNGAMDYWGQGTLVTVSS

SEQ ID NO: 43 - protein, 15 aa, synthetic construct
GGGGSGGGGSGGGGS

SEQ ID NO: 44 - protein, 16 aa, synthetic construct
GSGSGSGSGSGSGSGS

(19) United States
(12) Patent Application Publication
(10) Pub. No.: US 2026/0224701 A1
(43) Pub. Date: Aug. 6, 2026

(54) ANTIBODIES WITH SELECTIVITY FOR THE 4IG ISOFORM OF B7-H3 AND METHODS OF USE THEREOF

(71) Applicant: BOARD OF REGENTS, THE UNIVERSITY OF TEXAS SYSTEM, Austin, TX (US)

(72) Inventors: Seth T. GAMMON, Houston, TX (US); David PIWNICA-WORMS, Houston, TX (US)

(21) Appl. No.: 19/152,298

(22) PCT Filed: Jan. 30, 2024
(86) PCT No.: PCT/US2024/013584

§ 371 Date: Jul. 30, 2025

Related U.S. Application Data
(60) Provisional application No. 63/482,201, filed on Jan. 30, 2023.

(51) Int. Cl. A61K 40/31; A61K 9/00; A61K 38/00; A61K 40/42; A61P 35/00; C07K 14/705; C07K 16/28; C12N 5/16; C12N 15/11; G01N 33/575

(52) U.S. Cl. CPC A61K 40/31; A61K 9/0019; A61K 38/00; A61K 40/421; A61P 35/00; C07K 14/70503; C07K 16/2827; C12N 5/163; C12N 15/11; G01N 33/5758; C07K 2317/14; C07K 2317/24; C07K 2317/565; C07K 2317/92; C12N 2510/00

(57) ABSTRACT

Provided herein are antibodies and antigen binding portions thereof that specifically bind the 4Ig isoform of B7 homolog 3 protein (B7-H3), various compositions of such antibodies or antigen binding portions thereof, and recombinant nucleic acids encoding the antibodies and antigen binding portions thereof. Also provided are methods of using the antibodies or antigen binding portions thereof in therapeutics and diagnostics for cancer or inflammation.

### CROSS REFERENCE TO RELATED APPLICATIONS

This application claims priority to and the benefit of U.S. Provisional Patent Application Ser. No. 63/482,201, filed Jan. 30, 2023, the contents of which are incorporated herein by this reference as if fully set forth herein.

### REFERENCE TO A SEQUENCE LISTING SUBMITTED AS XML VIA EFS-WEB

The instant application contains a Sequence Listing in XML format. The Sequence Listing, named 090723-1424932-22-107PCT-SequenceListing.xml was created on Oct. 24, 2025, is 49,765 bytes in size, and is hereby incorporated by reference in its entirety.

### BACKGROUND

Immunotherapy aimed at suppressing immune checkpoints on effector T cells has enabled dramatic and sustained tumor response for selected solid tumors. Antibody-based immunotherapies have prompted the identification of clinically relevant tumor antigens that can serve as targets in solid tumors. Among them, the B7 ligand family, represents an attractive target for antibody-based immunotherapy as it is overexpressed on differentiated malignant cells and cancer-initiating cells, with limited heterogeneity and high frequency and in many different cancer types, with limited to low expression levels in normal tissue. However, because antigens, such as the B7 family, have different isoforms that function in distinctly different roles, an antibody with high specificity for a selected isoform is necessary. Different isoforms of the B7 family of antigens also have been identified in different inflammatory conditions. An antibody having high specificity for a select isoform is critical for maximizing the therapeutic and/or diagnostic efficacy. Thus, novel antibodies for immunotherapy approaches are needed to further improve clinical outcome in patients.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, provided herein are isolated antibodies or antibody fragments that specifically bind to B7-H3. In some embodiments, the isolated antibody or antibody fragment is a humanized antibody or antibody fragment. In some embodiments, the isolated antibody or antibody fragment comprises a heavy chain variable region (VH) having at least 90% identity to SEQ ID NO: 3 and comprising a VHCDR1 amino acid sequence comprising SEQ ID NO: 9, 15, or 21, a VHCDR2 amino acid sequence comprising SEQ ID NO: 10, 16, or 22, and a VHCDR3 amino acid sequence comprising SEQ ID NO: 11, 17, or 23, and comprising a tryptophan at position 47, a methionine at position 48, a valine at position 68, and an arginine at position 72 of SEQ ID NO: 3; and a light chain variable region (VL) having at least 90% identity to SEQ ID NO. 4 and comprising a VLCDR1 amino acid sequence comprising SEQ ID NO: 12, 18, or 24, a VLCDR2 amino acid sequence comprising SEQ ID NO: 13, 19 or 25, and a VLCDR3 amino acid sequence comprising SEQ ID NO. 14, 20, or 26, and having an arginine at position 45 and a tyrosine at position 70 of SEQ ID NO: 4.

In some embodiments, the isolated antibody or antibody fragment thereof comprises a light chain variable sequence as set forth in SEQ ID NO: 4. In some embodiments, the isolated antibody or antibody fragment thereof comprises a heavy chain variable sequence as set forth in SEQ ID NO: 3. In some embodiments, the humanized monoclonal antibody or antibody fragment comprises a heavy chain variable sequence as set forth in SEQ ID NO: 3 and a light chain variable sequence as set forth in SEQ ID NO: 4.

In some embodiments, the isolated antibody or antibody fragment comprises a heavy chain variable region (NH) having at least 95% identity to SEQ ID NO: 1 or SEQ ID NO: 2, and comprising a VHCDR1 amino acid sequence comprising SEQ ID NO: 9, 15, or 21, a VHCDR2 amino acid sequence comprising SEQ ID NO: 10, 16, or 22, and a VHCDR3 amino acid sequence comprising SEQ ID NO: 11, 17, or 23; and a light chain variable region (VL) having at least 95% identity to SEQ ID NO: 4, and comprising a VLCDR1 amino acid sequence comprising SEQ ID NO: 12, 18, or 24, a VLCDR2 amino acid sequence comprising SEQ ID NO: 13, 19 or 25, and a VLCDR3 amino acid sequence comprising SEQ ID NO: 14, 20, or 26, and having an arginine at position 45 and a tyrosine at position 70 of SEQ ID NO: 4. In some embodiments, the isolated antibody or antibody fragment comprises a heavy chain variable sequence as set forth in SEQ ID NO: 1 and a light chain variable sequence as set forth in SEQ ID NO: 4. In other embodiments, the isolated antibody or fragment thereof comprises a heavy chain variable sequence as set forth in SEQ ID NO: 2 and a light chain variable sequence as set forth in SEQ ID NO: 4.

In some embodiments, the isolated antibody or antibody fragment thereof is a monovalent scFv (single chain fragment variable) antibody, divalent scFv, Fab fragment, F(ab′)2 fragment, F(ab′)3 fragrant, Fv fragment, or single chain antibody. In some embodiments, the isolated antibody or antibody fragment thereof is a chimeric antibody, bispecific antibody, trispecific or multispecific antibody, or BiTE. In some embodiments, the isolated antibody or antibody fragment thereof is an IgG antibody or a recombinant IgG antibody or antibody fragment.

In some embodiments, the isolated antibody or antibody fragment competes for binding to the same epitope as the humanized antibody or an antibody fragment disclosed herein. In some embodiments, the isolated antibody or antibody fragment binds to an epitope on B7-H3 recognized by the isolated antibody or antibody fragment disclosed herein. In some embodiments, the isolated antibody or antibody fragment has increased binding affinity for the 4Ig isoform of B7-H3. In some embodiments, the isolated antibody or antibody fragment has increased specificity for the 4Ig isoform of B7-H3 over the 2Ig isoform of B7-H3. In certain embodiments, the isolated antibody or antibody fragment has at least 350-fold increased selectivity for the 4Ig isoform of B7-H3 over the 2Ig isoform of B7-H3.

In some embodiments, the isolated antibody or antibody fragment are conjugated or fused to an imaging agent, a cytotoxic agent, a metal, or a radioactive moiety. In some embodiments, the imaging agent is a fluorophore. In some embodiments, the radioactive moiety is Zr-89, Cu-64, F-18, Y-90, Lu-177, Tb-161, At-211, Ac-225, or Pb-212. In some embodiments, the antibodies or antibody fragments are immune conjugates or radio-immune conjugates. In some embodiments, the antibodies or antibody fragments are conjugated to flagellin or a flagellin derivative.

In some embodiments, the isolated antibodies or antibody fragments are antibody-drug conjugates.

In another aspect, provided herein are isolated nucleic acids encoding the antibody heavy and/or light chain variable regions of the isolated antibody of any of the disclosed embodiments. In some embodiments, the nucleic acids comprise a nucleotide sequence that is at least 90% identical to SEQ ID NO: 30. In some embodiments, the nucleic acids comprise a nucleotide sequence that is at least 90% identical to SEQ ID NO: 29. In some embodiments the nucleic acid sequence encoding the heavy chain variable region comprises SEQ ID NO: 29. In some embodiments, the nucleic acid sequence encoding the light chain variable region comprises SEQ ID NO: 30.

In some embodiments, provided herein are expression vectors comprising the nucleic acids of any one of the disclosed embodiments.

In some embodiments, provided herein are hybridoma or engineered cells comprising the nucleic acids encoding the antibodies or antibody fragments of any one of the disclosed embodiments.

In another aspect, provided herein are methods of making humanized monoclonal antibodies or antibody fragments of any one of the present embodiments, the methods comprising culturing the hybridomas or engineered cells of the present embodiments under conditions that allow expression of the antibody and optionally isolating the antibody from the culture.

In a further aspect, provided herein are chimeric antigen receptor (CAR) proteins comprising an antigen binding domain comprising a heavy chain variable region (VH) comprising VHCDR1, VHCDR2, and VHCDR3 amino acid sequences from any isolated antibody or antibody fragment disclosed herein; and a light chain variable region (VL) comprising VLCDR1, VLCDR2, and VLCDR3 amino acid sequences from any isolated antibody or antibody fragment disclosed herein.

In some embodiments, the antigen binding domain comprises heavy and light chain CDR sequences as follows: a heavy chain variable region sequence as set forth in SEQ ID NO: 3 or a heavy chain variable region having a VHCDR1 amino acid sequence comprising SEQ ID NO: 9, 15, or 21, a VHCDR2 amino acid sequence comprising SEQ ID NO: 10, 16, or 22, and a VHCDR3 amino acid sequence comprising SEQ ID NO: 11, 17, or 23; and comprising a tryptophan at position 47, a methionine at position 48, a valine at position 68, and an arginine at position 72 of SEQ ID NO: 3; and a light chain variable sequence as set forth in SEQ ID NO: 4 or a light chain variable region having a VLCDR1 amino acid sequence comprising SEQ ID NO: 12, 18, or 24, a VLCDR2 amino acid sequence comprising SEQ ID NO: 13, 19 or 25, and a VLCDR3 amino acid sequence comprising SEQ ID NO: 14, 20, or 26, and having an arginine at position 45 and a tyrosine at position 70 of SEQ ID NO: 4. In some embodiments, the antigen binding domain comprises a heavy chain variable sequence having at least 90% identity to SEQ ID NO: 1, SEQ ID NO: 2, or SEQ ID NO: 3, and a light chain variable sequence having at least 90% identity to SEQ ID NO: 4, and having an arginine at position 45 and a tyrosine at position 70 of SEQ ID NO: 4. In some embodiments, the antigen binding domain comprises a heavy chain variable sequence having at least 95% identity to SEQ ID NO: 1, SEQ ID NO: 2, or SEQ ID NO: 3, and a light chain variable sequence having at least 95% identity to SEQ ID NO: 4, and having an arginine at position 45 and a tyrosine at position 70 of SEQ ID NO: 4. In some embodiments, the antigen binding domain comprises a heavy chain variable sequence of SEQ ID NO: 1, SEQ ID NO: 2, or SEQ ID NO: 3 and a light chain variable sequence of SEQ ID NO: 4, and having an arginine at position 45 and a tyrosine at position 70 of SEQ ID NO: 4. In some embodiments, the antigen binding domain comprises a heavy chain variable sequence of SEQ ID NO: 3 and a light chain variable sequence of SEQ ID NO: 4.

In some embodiments, the antigen binding domain specifically binds to B7-H3. In some embodiments, the antigen binding domain specifically binds to the 4Ig isoform of B7-H3. In some embodiments, the antigen binding domain is a humanized antigen-binding domain.

In some embodiments, the CAR proteins further comprise a hinge domain, a transmembrane domain, and an intracellular signaling domain. In some embodiments, the CAR proteins further comprise the hinge domain wherein the hinge domain is a CD8a hinge domain or an IgG4 hinge domain. In some embodiments, the transmembrane domain is a CD8a transmembrane domain or a CD28 transmembrane domain. In some embodiments, the intracellular signaling domain comprises a CD3ζ intracellular signaling domain.

In another aspect, provided herein are nucleic acid molecules encoding a CAR of any of the present embodiments. In some embodiments, the sequence encoding the CAR is operatively linked to expression control sequences. In some embodiments, the nucleic acids are further defined as expression vectors.

In some embodiments, provided herein are engineered cells comprising a nucleic acid molecule encoding a CAR of any one of the disclosed embodiments. In some embodiments, the cell is a T cell. In some embodiments, the cell is an NK cell. In some embodiments, the nucleic acid is integrated into a genome of the cell. In some embodiments, the cell is a human cell.

In another aspect, provided herein are pharmaceutical compositions comprising a population of cells in accordance with any one of the present embodiments in a pharmaceutically acceptable carrier. In other aspects, provided herein are pharmaceutical compositions comprising an isolated antibody or antibody fragment as disclosed herein in a pharmaceutically acceptable carrier. In some embodiments, the antibody or antibody fragment is conjugated or fused to a cytotoxic agent, a metal, a radioactive moiety, or a drug.

In a further aspect, provided herein are methods of treating cancer in a human patient in need thereof. In some embodiments, the methods comprise administering to the patient an anti-tumor effective amount of a cell therapy comprising one or more cells in accordance with any one of the disclosed embodiments. In some embodiments, the cells are allogeneic cells. In some embodiments, the cells are autologous cells. In some embodiments, the cells are HLA matched to the human subject. In other embodiments, the methods comprise administering to the patient an anti-tumor effective amount of an isolated antibody or antibody fragment as disclosed herein. In some embodiments, the antibody or antibody fragment is conjugated or fused to a cytotoxic agent, a metal, a radioactive moiety, or a drug. In some embodiments, the cancer has been determined to express an elevated level of B7-H3 relative to a healthy tissue. In some embodiments, the cancer is a renal cancer, a pancreatic cancer, a colorectal cancer, a non-small cell lung cancer, an ovarian cancer, a bladder cancer, a melanoma, a prostate cancer, a breast cancer, a glioma, a lymphoma, or a neuroectodermal cancer. In some embodiments, the patient has previously failed to respond to an immune checkpoint inhibitor. In some embodiments, the patient has relapsed.

In some embodiments, the method further comprises administering at least a second anti-cancer therapy. In some embodiments, the second anti-cancer therapy is a chemotherapy, molecular targeted therapy, immunotherapy, radiotherapy, radioimmunotherapy, phototherapy, gene therapy, surgery, hormonal therapy, epigenetic modulation, anti-angiogenic therapy or cytokine therapy.

In another aspect, provided herein are methods for diagnosing a patient having cancer. In some embodiments, the methods comprise the detection and/or quantitation of B7-H3 using a B7-H3 antibody or antigen binding fragment as disclosed herein. In some embodiments, the B7-H3 antibody or antigen binding fragment specifically binds the 4Ig isoform of B7-H3. In some embodiments, the B7-H3 antibody or antigen binding fragment is conjugated to a bioluminescent or chemiluminescent label, a metal, or a radioisotope. In some embodiments, the methods are used for selecting a patient having cancer for treatment if the 4Ig isoform of B7-H3 is expressed by the cancer.

In yet another aspect, provided herein are methods of treating inflammation in a human patient in need thereof. In some embodiments, the methods comprise treating a patient with a therapeutically effective amount of a cell therapy or an antibody therapy as disclosed herein. In some embodiments, the antibody is a B7-H3 antibody as described herein. In some embodiments, the method further comprises administering at least a second therapeutically effective treatment for inflammation. In some embodiments, the second therapeutically effective therapy is a molecular targeted therapy, such as a drug, or other anti-inflammatory agents. In some embodiments, the disclosed B7-H3 antibodies and fragments thereof can be combined with corticosteroids, DMARDs, anti-cytokine therapies, or a combination thereof. In some embodiments, the disclosed B7-H3 antibodies and fragments thereof can be combined with immuno-inhibitory doses of radioisotopes. For example, the radioisotopes can include Zr-89, Cu-64, F-18, Y-90, Lu-177, Tb-161, At-211, Ac-225, or Pb-212. In some embodiments, immune inhibitory Fc domains may be coupled to CDRs described herein. In certain embodiments, the enhanced Fc domains may enhance killing of target immune cells.

In another aspect, provided herein are methods for diagnosing a patient having inflammation. In some embodiments, the methods comprise the detection and/or quantitation of B7-H3 using a B7-H3 antibody or antigen binding fragment as disclosed herein. In some embodiments, the B7-H3 antibody or antigen binding fragment specifically binds the 4Ig isoform of B7-H3. In some embodiments, the B7-H3 antibody or antigen binding fragment is conjugated to a bioluminescent or chemiluminescent label, a metal, or a radioisotope. In some embodiments, the methods are used for selecting a patient having inflammation for treatment if the 4Ig isoform of B7-H3 is expressed by the sample.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a panel of exemplary surface plasmon resonance (SPR) sensor-grams for binding affinity of 11 clones preselected from fast screening for expression, biophysical-properties, and affinity (Fab-SASA) screening and sensor-grams for relative controls.

FIG. 2 is an image of a Western blot analysis of reduced and non-reduced pre-selected antibodies as described herein.

FIG. 3 is panel of exemplary sensor-grams from the preselected humanized antibody clones.

FIG. 4 is a sequence alignment showing the framework mutations present in MIL33B-H1, MIL33B-H2, and MIL33B-H3 humanized antibodies. The sequence of the MIL33B-H3 humanized antibody is shown with a box.

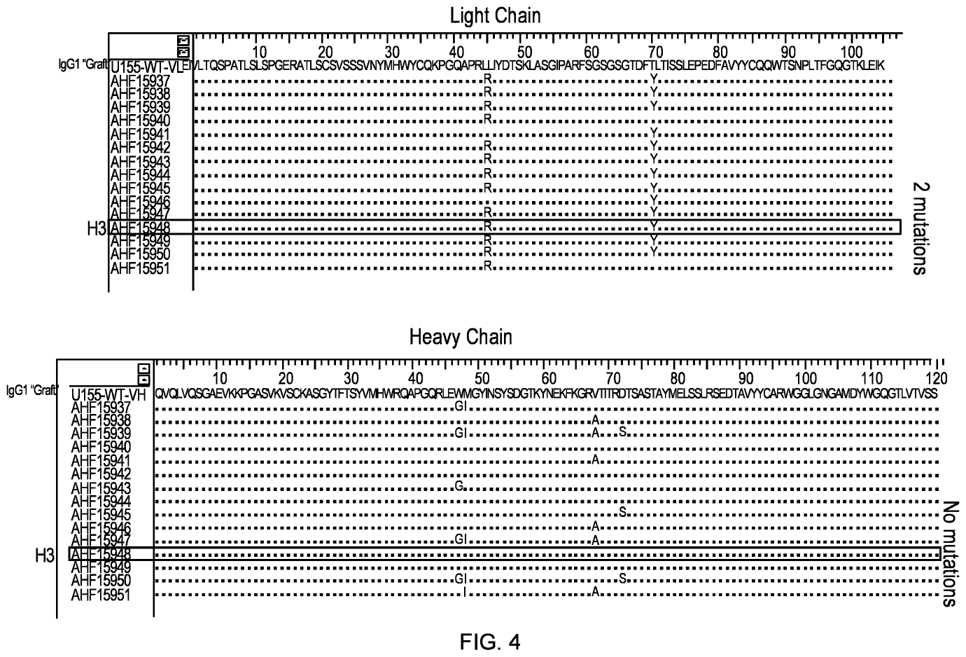

FIG. 5A is an image of Western blot analysis of 4Ig-B7-H3 and 2Ig-B7-H3 expression in non-activated leukemia monocytic cells (THP-1) in M0, M1, M2a, and M2c-like states and phorbol myristate acetate (PMA) activated THP-1 cells in M0, M1, M2a, and M2c-like states. FIG. 5B is a panel of microscopy images of immunofluorescence staining of THP-1 cells in M0, M1, M2a, and M2c-like states and treated with (bottom panels) or without (top panels) PMA and further incubated with MIL33B antibody.

FIG. 6 provides a panel of images of THP-1 cells in an undifferentiated state, treated with lipopolysaccharide (LPS) and Interferon-γ (INF-γ) to induce an M1-differentiated state, and THP-1 cells in an M1-differentiated state treated with PMA.

FIG. 7A provides a panel of images of SGM3 mice intraperitoneally injected with L-012 sodium salt in saline and pre-imaged (Day 0) and subsequently imaged following 3 days of incubation (day 3). FIG. 7B is a graph of the head reactive oxygen and nitrogen species (RONS) bursts following 5 days of treatment in SGM3 mice injected intraperitoneally with L-012 sodium salt in saline. M1-M4 represent the values for the four mice.

FIG. 8A provides a panel of SGM3 mice injected intraperitoneally with 200 μg of humanized hMIL33B-H3 in phosphate buffer saline (PBS) two times per week for 14 days or SGM3 mice injected intraperitoneally with PBS buffer as a control group. FIG. 8B provides a graph of the head RONS bursts in SGM3 mice at Day 0, Day 7, and Day 14. The slope of the line for mice treated with hMIL33B-H3 is −4823 while in the control group the slope is −201.

FIG. 9A is a graph of the head RONS bursts in SGM3 mice post treatment with the hMIL33B-H3 antibody or PBS for 14-days and imaged via L-012. Treatment ended on day 16 and SGM3 mice were imaged on days 21 and 28. The slope of the line for mice treated with hMIL33B-H3 is 8771 while in the control group the slope is −1043. FIG. 9B is a graph of the head RONS bursts following re-treatment with the hMIL33B-H3 antibody (triangles) and with PBS (circles). The slope of the line for mice treated with hMIL33B-H3 antibody is −15143 while in the control group the slope is −749.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Terminology

Unless otherwise defined, all terms of art, notations, and other scientific or medical terms or terminology used herein are intended to have the meanings commonly understood by those of ordinary skill in the art. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e., at least one) of the grammatical object of the article. By way of example, “an element” means at least one element and can include more than one element.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of those certain elements.” As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, for example, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Numerical quantities given herein are approximate unless stated otherwise, meaning that the term “about” or “approximately” can be inferred when not expressly stated. When “about” is applied to the beginning of a numerical range, it applies to both ends of the range.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L and D amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

Other terms used in the fields of recombinant nucleic acid technology, microbiology, immunology, antibody engineering, and molecular and cell biology as used herein will be generally understood by one of ordinary skill in the applicable arts.

### II. Introduction

B7-H3 (also referred to as CD276) is a co-inhibitory ligand expressed on the surface of many tumor cells as well as in the tumor microvasculature (Suh et al., Nat. Immunol., 4:899-906, (2003); Zang et al., Proc. Natl. Acad. Sci. U.S.A., 104:19458-63, (2007); Wang et al., Islets, 4:284-95 (2012)). It is thought to actively inhibit the effector functions of cytotoxic T lymphocytes (CTLs) or induce the generation of regulatory T cells, all of which down-regulate immune responses (Pardall, Nat. Rev. Cancer, 12:252-64 (2012)). Although CTLA-4 and B7-H3 are both members of the extended CD28/B7 family, CTLA-4 and B7-H3 have non-overlapping functions, and studies conducted in animal models suggest that the two pathways play distinct roles in immune regulation (Zang et al., 2007; Wang et al., 2012).

B7-H3 protein is expressed on most tumor cell types as well as tumor-associated vasculature (Seaman et al., Cancer Cell, 31:501-515 (2017)). For example, B7-H3 is overexpressed on renal, pancreatic, colorectal, non-small cell lung, ovarian, bladder, melanoma, and neuroectodermal cancers (Loo et al., Clin. Cancer Res., 18:3834-45 (2012)), as well as prostate cancer cells (Zang et al., 2007; Koenig, Medicographia, 36:285-92 (2014)), pointing to the broad applicability of targeting B7-H3 for therapy and imaging. For example, in prostatectomy specimens from 803 patients with localized disease, the vast majority (93%) of the prostate tumors expressed B7-H3 (Zang et al., 2007). Furthermore, high level expression of B7-H3 (and/or B7-H4, another co-inhibitory ligand) was associated with higher risk of clinical failure (metastases) and of death within 7 years, implicating these molecules as inhibitory immune checkpoints that act to suppress anti-tumor immune responses (Zang et al., 2007; Zang et al., Proc. Natl. Acad. Sci. U.S.A., 100:10388-92 (2003)). In addition, renal, melanoma, glioblastoma, thyroid, and pancreatic cancers show up to 99% positive staining for B7-H3 by IHC (Koenig, 2014). Most significantly, there is limited B7-H3 protein in normal human tissues (Koenig, 2014; Zang et al., 2003). Because B7-H3 is highly expressed on the surface of cancer cells as well as cancer vasculature, but not normal tissues, B7-H3 provides an excellent target for anti-cancer immuno-therapy, positron emission tomography (PET) and immuno-PET imaging, and bifunctional-conjugate drug therapy. B7-H3 also has been identified on cells in inflammation (See, e.g., Chen et al., Cell. Immunol. 352:104077 (2020); Sun et al., J. Immunol. Res., 2017, Article ID 5728512 (2017); Hashimoto et al., Bone Marrow Transplantation 56:2336-54 (2021))

B7-H3 is a type 1 membrane protein based upon the existence of an extracellular N-terminal signal sequence, and there is one predicted alpha helix consistent with a transmembrane protein. Both the 4Ig isoform and the 2Ig isoform of B7H3 are type 1 proteins (Zhou & Jin, Front Immunol 12:701006 (2021)). Published localization and staining with a variety of antibodies is consistent with a membrane protein and is consistent with other paralogues, including PD-L1.

The extracellular domain of many type 1 membrane proteins is frequently the target of therapeutic antibodies as it is the most accessible from the blood and interstitial spaces, but extracellular domains are susceptible to further processing, including proteolytic cleavage and shedding (Tsumagari et al., iScience, 24(4): 102259 (2021); Lichtenthaler et al., EMBO J, 37(15) (2018)). This presents two challenges: (1) antigen loss, which is difficult to address, and (2) the shed extracellular domain is now released into extracellular spaces and potentially into the circulation. In the case of B7-H3, there is a clearly reported circulating protein that is most consistent with the extracellular domain of the 2Ig isoform (Zhang et al., Immunology 123(4):538-46 (2008); Baral et al., Oncol Lett 8(3):1195-1201 (2014)). This circulation fragment increases in both inflammation and dramatically increases when tumors are present. Furthermore, it has been hypothesized that the 4Ig isoform of B7-H3, primarily found on tumor cells, may be resistant to proteolytic cleavage (Zhang et al., Bioengineered 12(2):11987-12002 (2021); Sun et al., PLoS One 6(9):e24751 (2011); Digregorio et al., Acta Neuropathol Commun, 9(1):59 (2021)).

Tumor-targeting antibodies that also target circulating proteins and protein isoforms have significant challenges due to a decrease in overall efficacy and may have an increase in side effects. The first challenge is delivery of the antibody and the antibody payload to the tumor target. Whether delivered by intravenous or subcutaneous routes, the blood pool provides the primary route of delivery of antibodies to the tumor compartment. Thus, if the antibody binds tightly to a circulating antigen, it is effectively “blocked” from immediate delivery to the tumor target. If the off-rate of the tumor/circulating antigen complex is faster than the excretion rate of the complex, then there could be some ability to target the tumor compartment. However, if the off-rate of the complex is slower than the complex clearance rate, then this effectively represents a “sink” for the antibody, and it will never be able to target the tumor compartment. Furthermore, small antibody-antigen complexes (~1:1 ratios) are cleared faster than typical non-complexed antibodies due to the engagement of “low affinity” FcRγ receptors in the liver and possibly in the kidney (Schlondorff & Banas, J. Am. Soc. Nephrol. 20(6):1179-87 (2009); Ganesan et al., J. Immunol., 189(10):4981-8 (2012)). This “sink” problem is particularly relevant for low dose, high efficacy ADCs, large circulating antigen loads, and high molar activity radiotracers and radio-therapeutics (low mass tracers). One of the first generation antibodies targeting B7-H3, known as 8H9, preferentially binds the extracellular domain of the 2Ig isoform of B7-H3, the most compatible with the circulating isoform (Ahmed et al., J. Biol. Chem. 290(50):30018-29; 2015)). 8H9 has failed in whole body systemic treatment models but has been successful as a therapeutic by direct injection of tumors in the brain or CNS wherein the blood pool can be bypassed (Kramer et al., J. Neurooncol. 97(3):409-18 (2010)).

The second challenge is the potential for increased toxicity. For antibodies injected at significant mass, the first consideration is the formation, tissue deposition, and reactivity toward medium and larger antibody-immune complexes. This occurs when antibodies bind antigen in excess of 1:1, e.g., 2:1 or higher. These larger complexes can exit circulation and generate local reactive immune responses, typically initiated by innate immune response downstream of FcRγ engagement, and can damage local tissues and in extrema cause significant adverse events (Rojko et al., Toxicol. Pathol. 42(4):725-64 (2014); Mayadas et al., Circulation 120(20):2012-24 (2009). With the advent of immune checkpoint therapy that enhances immune responses, this may be even more important. Indeed, it is interesting to note that the ADCC- or FcRγ-enhanced commercial antibody from Macrogenics, Inc., that targets the extracellular domain of B7-H3 (4Ig vs 2Ig selectivity not publicly available), demonstrated large therapeutic index as a single agent, but in the context of combined PD1 therapy that enhances immune response, had significant adverse events. Thus, it is important to maximize the selectivity between the tumor antigen and the circulating antigen isoform, if possible.

Known anti-B7-H3 antibodies demonstrate modest nanomolar affinity for only human or only mouse B7-H3 with limited specificity for the 4Ig as compared to the 2Ig isoform. The structure of the two isoforms are extremely similar with the exception that 4Ig B7-H3 has an extra IgC-IgV immunoglobulin domain in the extracellular region. The extracellular domain of the 2Ig isoform is an excellent model for the circulating form of B7-H3 found in the blood of patients with solid tumors and to a lesser extent the cerebral spinal fluid of patients with active CNS tumors/inflammation. For both radioisotope applications and antibody-drug conjugates (ADCs), binding of an antibody to the circulating form of B7-H3 can lead to less antibody payload delivered to the tumor target through compartmental binding competition and small immune complex clearance. In addition, for the reagent masses typically injected for ADC therapy, binding to soluble isoforms could further form medium to large immune complexes, leading to delivery of ADC payloads to cells expressing low affinity Fc receptors in the body, particularly in the liver, kidney and bone marrow, and general immuno-toxicity due to tissue deposition of medium to large immune complexes, including type III hypersensitivity reactions and coagulopathies. The combination of high affinity to the human 4Ig-B7-H3 target and low affinity to 2Ig-B7-H3 may therefore be important for maximizing the therapeutic index during systemic treatment of solid tumors with this humanized antibody, antibody conjugates, or fragments thereof, particularly when later combined with anti-PD1 or other immune stimulating immunotherapies. Preferred humanized monoclonal antibodies would display high specificity for the 4Ig isoform over the 2Ig isoform.

MIL33B is a murine antibody that specifically binds B7-H3 (described in PCT Publication No. WO 2021/101991). The CDRs of MIL33B yielded sub-nanomolar affinity toward the extracellular domain of the 4Ig isoform of B7-H3, the isoform most associated with tumor cells. The initial selectivity toward the extracellular domain of the 4Ig isoform over the 2Ig isoform, a model of the circulating form, was about 10-fold. This was indeed sufficient selectivity to enable tumor and antigen-specific targeting in murine tumors. During the humanization process of antibodies, the affinity toward the primary target can drop, and an affinity maturation process is then performed. Given that this process may involve the generation of a random library in and around the CDR, there are opportunities to screen for additional properties besides rescue of affinity (Liang et al., Sci Rep. 11(1):22365 (2021); Fujiwara et al., Biochem. Biophys. Res. Commun. 527(2):350-357 (2020)). Indeed, during the engraftment of the previously identified CDRs for MIL33B onto the human IgG1 ortholog, primary affinity for human B7-H3 was lost. As discussed in the examples, a library of point mutations was generated that included the framework region around the CDR that targeted the least conserved mutations from mouse framework to the human framework human. In selecting an antibody for further analysis and use, two additional criteria were used: (1) point mutations in the framework region were selected that provided maximum affinity to the extracellular domain of the human 4Ig isoform, and (2) in the case of a statistical tie, a point mutation in the framework region was selected that maximized the selectivity of human 4Ig over human 2Ig extracellular domain, in order to minimize the targeting of the circulating component. Surprisingly, as shown in the examples below, without changing the CDR sequence, but with changing from a murine antibody and framework to an optimized human framework, the selectivity for human 4Ig over human 2Ig extracellular domain increased from 10-fold selectivity to greater than 350-fold selectivity while maintaining sub nanomolar affinity to the 4Ig isoform. Consistent with this hypothesis, data for binding in HeLa cells, which express human B7-H3, improved. The humanized MIL33B-H3 antibody (also referred to herein as hMIL33B-H3) was superior to murine MIL33B for binding to live cells in binding assays despite the mild loss of receptor-binding affinity from 70 pM to 120 pM for the tumor surface 4Ig isoform.

As such, provided herein are antibodies and antigen binding portions thereof that specifically bind the 4Ig isoform of B7-H3 (or alternatively referred to as CD276). Also provided are various compositions of such antibodies or antigen binding portions thereof, recombinant nucleic acids encoding the antibodies and antigen binding portions thereof, and associated methods of use. The disclosed humanized MIL33B antibody has higher affinity than any commercialized antibody for human 4Ig-B7-H3. For moderate abundant targets, such as CD276, found on the tumor-immune microenvironment, high affinity is crucial for maximizing target binding. Furthermore, this humanized antibody has first-in-class selectivity for the 4Ig-B7-H3 extracellular domain found on human tumors as compared to the 2Ig-B7-H3 extracellular domain. This selectivity may be critical for systemic therapy, whereby the antibody must first bypass circulating soluble 2Ig-B7-H3 in order to target solid tumors, potentially maximizing payload delivery to the tumor while minimizing possible toxicity of immune complex formation, which includes coagulopathies. This selectivity also may be critical for targeting areas of inflammation in a subject.

### III. Antibodies

In one aspect, the present disclosure provides antibodies and antigen binding portions thereof that bind specifically to B7-H3. As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. As used herein, the term antibody also encompasses an antibody fragment, for example, an antigen binding fragment. Antigen binding fragments comprise at least one antigen binding domain. One example of an antigen binding domain is an antigen binding domain formed by a VH-VL dimer. Antibodies and antigen binding fragments can be described by the antigen to which they specifically bind.

The term “variable” is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework regions (FRs). The variable domains of native heavy and light chains each comprise four FRs, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity. Each VH and VL generally comprises three CDRs and four FRs, arranged in the following order (from N-terminus to C-terminus): FR1-CDR1-FR2-CDR2-FR3-CDR3-FR4. The CDRs are involved in antigen binding, and are generally thought to confer antigen specificity and binding affinity to the antibody. (See Kabat et al. (1991) Sequences of Proteins of Immunological Interest 5th ed., Public Health Service, NIH, Bethesda, MD.) CDR sequences on the heavy chain (VH) may be designated as CDRH1, 2, 3, while CDR sequences on the light chain (VL) may be designated as CDRL1, 2, 3.

The term “epitope,” as used herein, means a component of an antigen capable of specific binding to an antibody or antigen binding fragment thereof. Such components optionally comprise one or more contiguous amino acid residues and/or one or more non-contiguous amino acid residues. Epitopes frequently consist of surface-accessible amino acid residues and/or sugar side chains and can have specific three-dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an antigen binding protein binds can be determined using known techniques for epitope determination such as, for example, testing for antigen binding protein binding to antigen variants with different point mutations.

As used herein, the terms “binds specifically to,” “specific for,” “binds selectively to” and “selective for B7-H3 or an isoform or an epitope of a B7-H3 protein,” and the like, mean binding that is measurably different from a non-specific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

Provided herein are antibodies and antigen binding portions thereof that bind specifically to B7-H3. In some embodiments, the disclosed antibodies and fragments thereof bind specifically to the 4Ig isoform of B7-H3. The B7-H3 antibodies and antigen binding portions thereof are polypeptides. As used herein, the terms “antigen binding portion” and “fragment” are used interchangeably to refer to a portion of an antibody polypeptide sequence that binds specifically to B7-H3. B7-H3-specific antibodies were identified and tested as described in the Examples below. In some embodiments, the antibodies and antigen binding portions thereof provided herein are humanized antibody and antigen binding portions thereof. As discussed above, the antibodies and antigen binding portions thereof provided herein comprise specific mutations relative to MIL33B as described in PCT Publication No. WO 2021/101991 that result in increased selectivity for the 4Ig isoform of B7-H3 while maintaining sub-nanomolar affinity.

In one aspect, provided herein is an isolated antibody or antibody fragment, wherein the antibody or antibody fragment comprises: a heavy chain variable region (VH) having at least 90% identity (for example, at least 90%, 91%, 92%, 93%, 94%, 95%, 96%, 97%, 98%, or 99% identical) to SEQ ID NO: 3 and comprising a VHCDR1 amino acid sequence comprising SEQ ID NO: 9, 15, or 21, a VHCDR2 amino acid sequence comprising SEQ ID NO: 10, 16, or 22, and a VHCDR3 amino acid sequence comprising SEQ ID NO: 11, 17, or 23 and having a tryptophan at position 47, a methionine at position 48, a valine at position 68, and an arginine at position 72 of SEQ ID NO: 3; and a light chain variable region (VL) having at least 90% identity (for example, at least 90%, 91%, 92%, 93%, 94%, 95%, 96%, 97%, 98% or 99% identical) to SEQ ID NO: 4 and comprising a VLCDR1 amino acid sequence comprising SEQ ID NO: 12, 18, or 24, a VLCDR2 amino acid sequence comprising SEQ ID NO: 13, 19 or 25, and a VLCDR3 amino acid sequence comprising SEQ ID NO: 14, 20, or 26 and having an arginine at position 45 and a tyrosine at position 70 of SEQ ID NO: 4. The framework substitutions identified herein unexpectedly are shown to confer critical properties to the antibodies.

In some embodiments, heavy chain variable region sequences and light chain variable region sequences encompassed by this disclosure are set forth in Table 1. The CDR sequences in the variable domains listed in Table 1 are bold and underlined text. In some embodiments, the heavy chain variable region is encoded by a nucleotide sequence having at least 90% identity to any one of SEQ ID NOs: 27-29. In some embodiments, the light chain variable region is encoded by a nucleotide sequence having at least 90% identity to SEQ ID NO: 30.

TABLE 1
Antibody VH and VL amino acid sequences of selected clones.

| Antibody ID | VH sequence | VL sequence |
|---|---|---|
| MIL33B-H1 | SEQ ID NO: 1 | SEQ ID NO: 4 |
| (AHF 15945) | QVQLVQSGAEVKKPGASVKVSCKA | EIVLTQSPATLSLSPGERATLSCSVSS |
| | SGYTFTSYVMHWVRQAPGQRLEW | SVNYMHWYQQKPGQAPRRLIYDTS |
| | MGYINSYSDGTKYNEKFKGRVTITS | KLASGIPARFSGSGSGTDYTLTISSLE |
| | DTSASTAYMELSSLRSEDTAVYYCA | PEDFAVYYCQQWTSNPLTFGQGTK |
| | RWGGLGNGAMDYWGQGTLVTVS | LEIK |
| | S | |
| MIL33B-H2 | SEQ ID NO: 2 | SEQ ID NO: 4 |
| | QVQLVQSGAEVKKPGASVKVSCKA | EIVLTQSPATLSLSPGERATLSCSVSS |
| | SGYTFTSYVMHWVRQAPGQRLEW | SVNYMHWYQQKPGQAPRRLIYDTS |
| | MGYINSYSDGTKYNEKFKGRATITR | KLASGIPARFSGSGSGTDYTLTISSLE |
| | DTSASTAYMELSSLRSEDTAVYYCA | PEDFAVYYCQQWTSNPLTFGQGTK |
| | RWGGLGNGAMDYWGQGTLVTVS | LEIK |
| | S | |
| MIL33B-H3 | SEQ ID NO: 3 | SEQ ID NO: 4 |
| | QVQLVQSGAEVKKPGASVKVSCKA | EIVLTQSPATLSLSPGERATLSCSVSS |
| | SGYTFTSYVMHWVRQAPGQRLEW | SVNYMHWYQQKPGQAPRRLIYDTS |
| | MGYINSYSDGTKYNEKFKGRVTITR | KLASGIPARFSGSGSGTDYTLTISSLE |
| | DTSASTAYMELSSLRSEDTAVYYCA | PEDFAVYYCQQWTSNPLTFGQGTK |
| | RWGGLGNGAMDYWGQGTLVTVS | LEIK |
| | S | |

There are various software programs that can be used to identify predicted CDRs in a given antibody polypeptide sequence, including IMGT/DomainGapAlign (as described in Ehrenmann et al., Nucleic Acids Res. 38(Database issue):D301-D307 (2010); Ehrenmann & Lefranc, Cold Spring Harbor Protoc. 2011(6):737-49 (2011); Paratome (as described by Kunik et al., PLoS Comput Biok, 8(2):e1002388 (2012); Kunik et al., Nucleic Acids Res., 40 (Web Server issue):W521-4 (2012)); and Chothia. In certain embodiments, heavy chain CDR sequences encompassed by this disclosure are set forth in Tables 2-4. In certain embodiments, light chain CDR sequences encompassed by this disclosure are set forth in Tables 2-4.

TABLE 2
CDRs of variable sequences of the CD276 antibody as predicted by
IMGT/DomainGapAlign.

| Chain | CDR1 | CDR2 | CDR3 |
|---|---|---|---|
| Heavy | GYTFTSYV | INSYSDGT | ARWGGLGNGAMDY |
| | (SEQ ID NO: 9) | (SEQ ID NO: 10) | (SEQ ID NO: 11) |
| Light | SSVNY | DTS | QQWTSNPLT |
| | (SEQ ID NO: 12) | (SEQ ID NO: 13) | (SEQ ID NO: 14) |

TABLE 3
CDRs of variable sequences of the CD276 antibody as predicted by Paratome.

| Chain | CDR1 | CDR2 | CDR3 |
|---|---|---|---|
| Heavy | YTFTSYVMH | GIGYINSYSDGTKY | RWGGLGNGAMDY |
| | (SEQ ID NO: 15) | (SEQ ID NO: 16) | (SEQ ID NO: 17) |
| Light | SSVNYMH | RLIYDTSKLAS | QQWTSNPL |
| | (SEQ ID NO: 18) | (SEQ ID NO: 19) | (SEQ ID NO: 20) |

TABLE 4
CDRs of variable sequences of the CD276 antibody as predicted by Chothia.

| Chain | CDR1 | CDR2 | CDR3 |
|---|---|---|---|
| Heavy | GYTFTSY | NSYSDG | WGGLGNGAMD |
| | (SEQ ID NO: 21) | (SEQ ID NO: 22) | (SEQ ID NO: 23) |
| Light | SVSSSVNYMH | DTSKLAS | QQWTSNPLT |
| | (SEQ ID NO: 24) | (SEQ ID NO: 25) | (SEQ ID NO: 26) |

As used herein, the term chimeric antibody refers to an antibody that includes the heavy chain and light chain variable regions from one antibody (e.g., a murine antibody) and the remaining antibody sequence from a second antibody (e.g., human antibody). In some embodiments, the antibody or antigen binding fragment thereof may be generated from a chimeric antibody that has a heavy chain variable region comprising an amino acid sequence that is at least 90% identical (for example, at least 90%, 91%, 92%, 93%, 94%, 95%, 96%, 97%, 98% or 99% identical) to SEQ ID NOs: 31 and a light chain variable region that includes an amino acid sequence that is at least 90% identical (for example, at least 90%, 91%, 92%, 93%, 94%, 95%, 96%, 97%, 98% or 99% identical) to SEQ ID NO: 33.

As used herein, the term grafted antibody refers to a humanized antibody that includes the CDR sequences from one antibody (e.g., a murine antibody) and the remaining framework and remaining antibody sequence from a human antibody. In some embodiments, the antibody or antigen binding fragment thereof may be generated from a grafted antibody that has a heavy chain variable region comprising an amino acid sequence that is at least 90% identical (for example, at least 90%, 91%, 92%, 93%, 94%, 95%, 96%, 97%, 98% or 99% identical) to SEQ ID NOs: 32; and a light chain variable region that includes an amino acid sequence that is at least 90% identical (for example, at least 90%, 91%, 92%, 93%, 94%, 95%, 96%, 97%, 98% or 99% identical) to SEQ ID NOs: 34.

In some embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NOs: 1, 2, or 3; and a light chain variable region that includes an amino acid sequence that is at least 90% identical (for example, at least 90%, 91%, 92%, 93%, 94%, 95%, 96%, 97%, 98% or 99% identical) to SEQ ID NO: 4. In further embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NO: 3 and that comprises a tryptophan at position 47, a methionine at position 48, a valine at position 68, and an arginine at position 72 of SEQ ID NO: 3; and a light chain variable region that includes an amino acid sequence that is at least 90% identical (for example, at least 90%, 91%, 92%, 93%, 94%, 95%, 96%, 97%, 98% or 99% identical) to SEQ ID NO: 4 and that has an arginine substitution at position 45 and a tyrosine substitution at position 70 of SEQ ID NO: 4.

In some embodiments, the antibody or antigen binding fragment thereof has a heavy chain variable region comprising a CDRH3 comprising any one of SEQ ID NOs: 11, 17, or 23. In some embodiments, the antibody or antigen binding fragment thereof has a heavy chain variable region comprising a CDRH3 comprising any one of SEQ ID NOs: 11, 17, or 23 and comprising a tryptophan corresponding to position 47, a methionine corresponding to position 48, a valine corresponding to position 68, and an arginine corresponding to position 72 of SEQ ID NO: 3.

In some embodiments, the antibody or antigen binding fragment thereof has a heavy chain variable region comprising any of the CDR1, CDR2, or CDR3 sequences listed in Tables 2-4. In some embodiments, the antibody or antigen binding fragment thereof has a heavy chain variable region comprising any of the CDR1, CDR2, or CDR3 sequences listed in Tables 2-4 and has a tryptophan corresponding to position 47, a methionine corresponding to position 48, a valine corresponding to position 68, and an arginine corresponding to position 72 of SEQ ID NO: 3. In some embodiments, the antibody or antigen binding fragment thereof has a light chain variable region comprising any of the CDR1, CDR2, or CDR3 sequences listed in Tables 2-4. In some embodiments, the antibody or antigen binding fragment thereof has a light chain variable region comprising any of the CDR-1, CDR-2, or CDR-3 sequences listed in Tables 2-4 and has an arginine substitution corresponding to position 45 and a tyrosine substitution corresponding to position 70 of SEQ ID NO: 4.

In some embodiments, the antibody or antigen binding fragment thereof includes a heavy chain variable region comprising an amino acid sequence that is at least 90% identical (for example, at least 90%, 91%, 92%, 93%, 94%, 95%, 96%, 97%, 98% or 99% identical) to any of SEQ ID NOs: 1-3. In some embodiments, the antibody or antigen binding fragment thereof has a heavy chain variable region comprising an amino acid sequence that is at least 90% identical (for example, at least 90%, 91%, 92%, 93%, 94%, 95%, 96%, 97%, 98% or 99% identical) to SEQ ID NO: 3 and that comprises a tryptophan corresponding to position 47, a methionine corresponding to position 48, a valine corresponding to position 68, and an arginine corresponding to position 72.

The disclosure also provides an antibody or antigen binding portion thereof that specifically binds to B7-H3, wherein the antibody or antigen binding portion thereof comprises a heavy chain variable region comprising an amino acid sequence that is at least 90% identical (for example, at least 90%, 91%, 92%, 93%, 94%, 95%, 96%, 97%, 98% or 99% identical) to any of SEQ ID NOs: 1-3 and a light chain variable region comprising an amino acid sequence that is at least 90% identical (for example, at least 90%, 91%, 92%, 93%, 94%, 95%, 96%, 97%, 98% or 99% identical) to SEQ ID NO: 4. Table 1 provides the sequences for SEQ ID Nos: 1-4. In some embodiments, the antibody or antigen binding fragment thereof that specifically binds to B7-H3 has a heavy chain variable region comprising an amino acid sequence that is at least 90% identical (for example, at least 90%, 91%, 92%, 93%, 94%, 95%, 96%, 97%, 98% or 99% identical) to SEQ ID NOs: 3 and that comprises a tryptophan at position 47, a methionine at position 48, a valine at position 68, and an arginine at position 72 of SEQ ID NO: 3; and a light chain variable region that includes an amino acid sequence that is at least 90% identical (for example, at least 90%, 91%, 92%, 93%, 94%, 95%, 96%, 97%, 98% or 99% identical) to SEQ ID NOs: 4 and that has an arginine at position 45 and a tyrosine substitution at position 70 of SEQ ID NO: 4.

In some embodiments, the antibody or antigen binding portion thereof comprises a heavy chain variable region or a light chain variable region comprising an amino acid sequence that is at least 90% identical (for example, at least 90%, 91%, 92%, 93%, 94%, 95%, 96%, 97%, 98% or 99% identical) to any of the sequences in Tables 2-4 and wherein the heavy chain variable region comprises a tryptophan corresponding to position 47, a methionine corresponding to position 48, a valine corresponding to position 68, and an arginine corresponding to position 72; and the light chain variable region comprises an arginine corresponding to position 45 and a tyrosine corresponding to position 70 of SEQ ID NO: 3.

In some embodiments, the antibody or antigen binding portion thereof comprises a heavy chain variable region or a light chain variable region comprising an amino acid sequence that is at least 90% identical (for example, at least 90%, 91%, 92%, 93%, 94%, 95%, 96%, 97%, 98% or 99% identical) to any of the sequences in Tables 2-4 and wherein the light chain variable region comprises an arginine corresponding to position 45 and a tyrosine substitution corresponding to position 70 of SEQ ID NO: 4.

In each case, where a specific amino acid sequence is recited, embodiments comprising a sequence having at least 90% (e.g., 90%, 91%, 92%, 93%, 94%, 95%, 96%, 97%, 98%, or 99%) identity to the recited sequence are also provided.

The amino acid residue sequences provided herein are set forth in single-letter amino acid code which can be used interchangeably with three-letter amino acid code. An amino acid refers to any monomer unit that can be incorporated into a peptide, polypeptide, or protein. The twenty natural or genetically encoded alpha-amino acids are as follows: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). The structures of these twenty natural amino acids are shown in, e.g., Stryer et al., Biochemistry, 5^{th} ed., Freeman and Company (2002). The term amino acid also includes unnatural amino acids, modified amino acids (e.g., having modified side chains and/or backbones), and amino acid analogs.

The terms identical or percent identity, in the context of two or more nucleic acids or polypeptide sequences, refer to two or more sequences or subsequences that are the same or have a specified percentage of nucleotides or amino acid residues that are the same (e.g., at least 60%, at least 65%, at least 70%, at least 75%, at least 80%, at least 85%, at least 90%, or at least 95% or greater identity over a specified region), when compared and aligned for maximum correspondence over a comparison window, or designated region as measured using one of the following sequence comparison algorithms or by manual alignment and visual inspection.

As with all peptides, polypeptides, and proteins, including fragments thereof, it is understood that additional modifications in the amino acid sequence of the B7-H3-specific antibodies or antigen binding fragments thereof described herein, for example, in the heavy chain variable region and/or light chain variable region, can occur that do not alter the nature or function of the antibodies or antigen binding fragments thereof. Such modifications include conservative amino acids substitutions, such that each recited sequence optionally contains one or more conservative amino acid substitutions. The list provided below identifies groups that contain amino acids that are conservative substitutions for one another; these groups are exemplary as other conservative substitutions are known to those of skill in the art:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an aspartic acid at a specific residue is mentioned, also contemplated is a conservative substitution at the residue, for example, glutamic acid. Non-conservative substitutions, for example, substituting a proline with glycine or substituting a lysine with an asparagine, are also contemplated.

In some instances, the affinity of B7-H3-specific antibodies or antigen binding fragments thereof may be optimized through mutations to increase or decrease affinity as desired based on one or more of the known characteristics of the binding interaction with B7-H3, the structure of either or both of the antibodies or fragments thereof, or the B7-H3 protein. For example, in some embodiments, the antibodies or antigen binding fragments disclosed herein, that include certain conserved amino acids and certain amino acid substitutions, have increased affinity and specificity for the 4Ig isoform of B7-H3 as compared to the 2Ig isoform. In some instances, the mutations permit facile elution of purified antibodies or fragments thereof under desirable elution conditions during isolation and purification.

Methods of generating and screening for antibodies and antigen binding fragments thereof as provided in this disclosure are described in the Examples and are well-known in the art. Methods of further modifying antibodies for enhanced properties (e.g., enhanced affinity, chimerization, humanization) as well as generating antigen binding fragments, as described herein, are also well-known in the art.

In some embodiments, the heavy chain variable region and/or the light chain variable region of the isolated antibody or antibody fragment has an identical sequence to the heavy chain variable region and/or the light chain variable region of the antibody produced by the methods described herein and, in the Examples, below. In some embodiments, the heavy chain variable region and/or the light chain variable region of the isolated antibody comprises one or more modifications, e.g., amino acid substitutions, deletions, or insertions.

The heavy chain variable region sequence and/or light chain variable region sequence of an antibody described herein can be engineered to comprise one or more variations in the heavy chain variable region sequence and/or light chain variable region sequence. In some embodiments, the engineered variation(s) improves the binding affinity of the antibody for B7-H3. In some embodiments, the engineered variation(s) improves the binding affinity of the antibody for 4Ig isoform of B7-H3. In some embodiments, the engineered variation(s) decreases the cross-reactivity of the antibody for a second antigen.

In some embodiments, the engineered variation is a variation in one or more CDRs, e.g., an amino acid substitution in a heavy chain CDR and/or a light chain CDR as described herein. In some embodiments, the engineered variation is a variation in one or more framework regions, e.g., an amino acid substitution in a heavy chain framework region and/or a light chain framework region. In some embodiments, the engineered variation is a reversion of a region of the heavy chain and/or light chain sequence to the inferred naïve sequence. Methods for determining an inferred naïve immunoglobulin sequence are described in the art. See, e.g., Magnani et al., PLoS Negl Trop Dis, 2017, 11:e0005655, doi:10.1371/journal.pntd.0005655.

In some embodiments, affinity maturation is used to engineer further mutations that enhance the binding affinity of the antibody for B7-H3 or enhance the cross-reactivity of the antibody for a second antigen. Methods for performing affinity maturation are known in the art. See, e.g., Renaut et al., Methods Mol Biol, 2012, 907:451-61.

The present disclosure also encompasses antibodies or fragments thereof that bind to the same epitope of B7-H3 or the 4Ig isoform of B7-H3 as the antibodies disclosed herein. Such antibodies can be identified using routine techniques known in the art, including, for example, competitive binding assays.

The present disclosure also provides chimeric antibodies. The term chimeric antibody refers to an antibody in which a component of the heavy and/or light chain is derived from a particular source or species, while the remainder of the heavy and/or light chain is derived from a different source or species.

A human antibody is one that possesses an amino acid sequence corresponding to that of an antibody produced by a human or a human cell, or derived from a non-human source that utilizes a human antibody repertoire or human antibody-encoding sequences (e.g., obtained from human sources, genetically modified non-human sources, or designed de novo). Human antibodies specifically exclude humanized antibodies.

Humanized forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies can also comprise residues that are not found in either the recipient antibody or the donor antibody. Such modifications can be made to further refine antibody function. (See Jones et al. (1986) Nature, 321:522-525; Riechmann et al. (1988) Nature, 332:323-329; and Presta, (1992) Curr Op Struct Biol., 2:593-596). In some embodiments, the antibody or fragment thereof is a chimeric or grafted antibody encoded by nucleotide sequences set forth in Table 6.

In some embodiments, the antibody or antigen binding fragment thereof provided herein can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to as a half antibody). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv, for example), single variable domain antibodies, diabodies (Dab) (bivalent and bispecific), and chimeric (e.g., humanized) antibodies, which may be produced by the modification of whole antibodies or synthesized de novo using recombinant DNA technologies. These functional antibody fragments retain the ability to bind specifically to their respective antigen. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or an in vitro generated antibody. The antibody can have a heavy chain constant region chosen from, e.g., IgG1, IgG2, IgG3, or IgG4. The antibody can also have a light chain chosen from either kappa or lambda light chains.

As used herein, the term monoclonal antibody refers to an antibody from a population of substantially homogeneous antibodies. A population of substantially homogeneous antibodies comprises antibodies that are the same or substantially similar and that bind the same epitope(s), except for variants that can normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of yeast clones, phage clones, bacterial clones, mammalian cell clones, hybridoma clones, or other recombinant DNA clones. The selected antibody can be further altered, for example, to improve affinity for the target, for example, by affinity maturation, to humanize the antibody, to improve its production in cell culture, and/or to reduce its immunogenicity in a subject.

Antigen binding fragments of an antibody molecule are well known in the art, and include, for example, (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv) (see e.g., Bird et al. (1988) Science 242:423-426; Huston et al. (1988) Proc. Natl. Acad. Sci. USA 85:5879-5883); (viii) a single domain antibody. These antibody fragments are obtained using conventional techniques known to those skilled in the art, and the fragments are screened for utility in the same manner as are intact antibodies.

In some embodiments, the humanized monoclonal antibody comprises a heavy chain variable region sequence and a light chain variable region sequence that are derived from an immunoglobulin producing human B cell, and further comprises a kappa or lambda light chain constant region. In some embodiments, the light chain constant region (kappa or lambda) is from the same type of light chain (i.e., kappa or lambda) as the light chain variable region that was derived from the immunoglobulin producing human B cell; as a non-limiting example, if an IgE-producing human B cell comprises a kappa light chain, then the monoclonal antibody that is produced can comprise the light chain variable region from the IgE-producing B cell and further comprises a kappa light chain constant region.

In some embodiments, the humanized monoclonal antibody comprises a heavy chain variable region sequence and a light chain variable region sequence that are derived from an immunoglobulin-producing human B cell, and further comprises a heavy chain constant region having an IgG isotype (e.g., IgG4), an IgA isotype (e.g., IgA1), an IgM isotype, an IgD isotype, or that is derived from an IgG, IgA, IgM, or IgD isotype (e.g., is a modified IgG4 constant region). It will be appreciated by a person of ordinary skill in the art that the different heavy chain isotypes (IgA, IgD, IgE, IgG, and IgM) have different effector functions that are mediated by the heavy chain constant region, and that for certain uses it may be desirable to have an antibody that has the effector function of a particular isotype (e.g., IgG).

In some embodiments, the humanized monoclonal antibody comprises a native (i.e., wild-type) human IgG, IgA, IgM, or IgD constant region. In some embodiments, the monoclonal antibody comprises a native human IgG1 constant region, a native human IgG2 constant region, a native human IgG3 constant region, a native human IgG4 constant region, a native human IgA1 constant region, a native human IgA2 constant region, a native human IgM constant region, or a native human IgD constant region. In some embodiments, the monoclonal antibody comprises a heavy chain constant region that comprises one or more modifications. It will be appreciated by a person of ordinary skill in the art that modifications such as amino acid substitutions can be made at one or more residues within the heavy chain constant region that modulate effector function. In some embodiments, the modification reduces effector function, e.g., results in a reduced ability to induce certain biological functions upon binding to an Fc receptor expressed on an effector cell that mediates the effector function. In some embodiments, the modification (e.g., amino acid substitution) prevents in vivo Fab arm exchange, which can introduce undesirable effects and reduce the therapeutic efficacy of the antibody. See, e.g., Silva et al., J Biol Chem, 2015, 280:5462-5469.

In some embodiments, the humanized monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one or more modifications that modulate effector function. In some embodiments the monoclonal antibody comprises a human IgM constant region, human IgD constant region, human IgG constant region that is derived from IgG1, IgG2, IgG3, or IgG4, or human IgA constant region that is derived from IgA1 or IgA2. In some embodiments, the monoclonal antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one, two, three, four, five, six, seven, eight, nine, ten or more modifications (e.g., amino acid substitutions). In some embodiments the constant regions includes variations (e.g., one, two, three, four, five, six, seven, eight, nine, ten or more amino acid substitutions) that affect effector function.

In some embodiments the antibody with specified CDRs is an allotype other than the allotype(s) found associated with the antibodies produced by the methods described herein and, in the Examples, below. The antibody may comprise an allotype selected from those listed in Table 5 below, which is different from an allotype of antibodies produced by the methods described herein and, in the Examples, below. In some embodiments, the antibody may comprise any individual allotype selected from those listed in Table 5, with the proviso that the allotype differs from the corresponding allotype of antibodies produced by the methods described herein and, in the Examples, below.

TABLE 5
Human immunoglobulin allotypes.

| | Heavy chains | | | | |
|---|---|---|---|---|---|
| Isotype/type | IgG1 | IgG2 | IgG3 | IgA | Light chains |
| Allotypes | G1m | G2m | G3m | A2m | Km |
| | 1(a) | 23(n) | 21(g1) | 1 | 1 |
| | 2(x) | | 28(g5) | 2 | 2 |
| | 3(f) | | 11(b0) | | 3 |
| | 17(z) | | 5(b1) | | |
| | | | 13 (b3) | | |
| | | | 14 (b4) | | |
| | | | 10 (b5) | | |
| | | | 15(s) | | |
| | | | 16(t) | | |
| | | | 6(c3) | | |
| | | | 24(c5) | | |
| | | | 26(u) | | |
| | | | 27 (v) | | |

NB: Alphabetical notation given within brackets. From: Jefferis and Marie-Paule Lefranc, 2009, “Human immunoglobulin allotypes: Possible implications for immunogenicity” mAbs 1(4): 332-338, incorporated herein by reference.

In some embodiments, a humanized monoclonal antibody comprises CDR sequences, a heavy chain variable region, and/or a light chain variable region as described herein (e.g., as disclosed in Table 1) and further comprises a heavy chain constant region and/or a light chain constant region that is heterologous to the antibody produced by the methods described herein and, in the Examples, below from which the CDR sequences and/or variable region sequences are derived. For example, in some embodiments, the monoclonal antibody comprises the CDR sequences and/or variable region sequences of an antibody produced by the methods described herein and in the Examples below, and further comprises a heavy chain constant region and a light chain constant region that is heterologous to the antibody produced by the methods described herein and in the Examples below (e.g., the heavy chain constant region and/or light chain constant region is a wild-type or modified IgG1, IgG2, IgG3, or IgG4 constant region), or the heavy chain constant region and/or light chain constant region comprises one or more modifications (e.g., amino acid substitutions) relative to the native constant region of the antibodies produced by the methods described herein and in the Examples below.

The antibodies and fragments thereof of this disclosure may comprise variations in heavy chain constant regions to change the properties of the synthetic antibody relative to the corresponding naturally occurring antibody. Exemplary changes include mutations to modulate antibody effector function (e.g., complement-based effector function or FcγR-based effector function), alter half-life, modulate co-engagement of antigen and FcγRs, introduce or remove glycosylation motifs (glyco-engineering). See Fonseca et al., 2018, “Boosting half-life and effector functions of therapeutic antibodies by Fc-engineering: An interaction-function review” Int J Biol Macromol. 19:306-311; Wang et al., 2018, “IgG Fc engineering to modulate antibody effector functions” Protein Cell 2018, 9(1):63-73; Schlothauer, 2016, “Novel human IgG1 and IgG4 Fc-engineered antibodies with completely abolished immune effector functions,” Protein Engineering, Design and Selection 29(10):457-466; Tam et al., 2017, “Functional, Biophysical, and Structural Characterization of Human IgG1 and IgG4 Fc Variants with Ablated Immune Functionality” Antibodies 6, 12, each incorporated herein by reference for all purposes.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, rat, guinea, pig, human, camel, llama, fish, shark, goat, rabbit, and bovine. Single domain antibodies are described, for example, in International Application Publication No. WO 94/04678. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species (e.g., camel, llama, dromedary, alpaca, and guanaco) or other species besides Camelidae.

In some embodiments, an antigen binding fragment can also be or can also comprise, e.g., a non-antibody, scaffold protein. These proteins are generally obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is screened, and in accordance with the methods described herein, against a target antigen of interest to identify those variants having optimal selectivity and affinity for the target antigen. See, e.g., Hey et al. (2005) TRENDS Biotechnol. 23(10):514-522.

One of ordinary skill in the art would appreciate that the scaffold portion of the non-antibody scaffold protein can include, e.g., all or part of the Z domain of S. aureus protein A, human transferrin, human tenth fibronectin type III domain, kunitz domain of a human trypsin inhibitor, human CTLA-4, an ankyrin repeat protein, a human lipocalin (e.g., anticalins, such as those described in, e.g., International Application Publication No. WO2015/104406), human crystallin, human ubiquitin, or a trypsin inhibitor from E. elaterium.

Any of the B7-H3-specific antibodies or antigen binding fragments thereof described herein can be modified with covalent and/or non-covalent modifications. Such modifications can be introduced into the antibodies or antigen binding fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments. Recombinant techniques can be used to modify antibodies or antigen binding fragments thereof. For example, amino acids found to not contribute to either the activity or the binding specificity or affinity of the antibody can be deleted without a loss in the respective activity. Insertions, deletions, substitutions, or other selected modifications of particular regions or specific amino acids residues, provided the activity of the fragment is not significantly altered or impaired compared to the non-modified antibody, or antigen binding fragment thereof can be made. Such methods are readily apparent to a skilled practitioner in the art and can include site specific mutagenesis of the nucleic acid encoding the antibody or fragment thereof. (Zoller et al., Nucl. Acids Res. 10:6487-500 (1982)). In some instances, the B7-H3-specific antibodies or antigen binding fragments may be labeled by a variety of means for use in diagnostic and/or pharmaceutical applications.

In some embodiments, the antibodies or antigen binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. In some embodiments, the heterologous moiety is an antibody or antigen binding fragment thereof that specifically binds to a different target, and such a conjugated antibody is referred to as a bispecific antibody. Additional suitable heterologous polypeptides include, e.g., an antigenic tag (e.g., FLAG (DYKDDDDK) (SEQ ID NO: 35), polyhistidine (6-His; HHHHHH (SEQ ID NO: 36) or HEHEHE (SEQ ID NO: 37)), hemagglutinin (HA; YPYDVPDYA (SEQ ID NO: 38)), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase. Another labeling technique which may result in greater sensitivity consists of coupling the antibodies to low molecular weight haptens. These haptens can then be specifically altered by means of a second reaction. For example, it is common to use haptens such as biotin, which reacts with avidin, or dinitrophenol, pyridoxal, or fluorescein, which can react with specific antihapten antibodies. Additional acceptable heterologous moieties are described below in Section VIII.

In some instances, the B7-H3 antibody or antigen-binding fragment thereof may be conjugated to an imaging agent or radiotherapeutic agent. For example, the B7-H3 antibody or antigen-binding fragment thereof may be labelled for use in radionuclide imaging or radioligand therapy. In particular, the agent may be directly or indirectly labelled with a radioisotope. Examples of radioisotopes that may be used are: ²²⁵Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁴I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ²¹²Pb, ³²P, ³³P ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵S, ¹⁰⁵Ag, ²²Na, ²⁴Na, ⁸⁹Sr, ³⁵S, ³⁸S, ¹⁷⁷Ta, ¹⁶¹Tb, ⁹⁶Tc, ^{99m}Tc, ^{94m}Tc, ²⁰¹Tl, ²⁰²Tl, 113Sn, ^{117m}Sn, ¹²¹Sn, ¹⁶⁶Yb, ¹⁶⁹Yb, ¹⁷⁵Yb, ⁸⁸Y, ⁹⁰Y, ⁸⁶Zr, ⁸⁹Zr, ⁶²Zn and ⁶⁵Zn. Preferably the radioisotope is ²²⁵Ac, ²¹¹At, ¹³¹I, ¹²⁵I, ¹²⁴I, ¹²³I, ¹¹¹I, ¹⁷⁷Lu, ^{99m}Tc, ⁹⁰Y, ¹⁸⁶Re, ¹⁸⁸Re, ³²P, ¹⁵³Sm, ⁸⁹Zr, ⁶⁸Ga, ⁷⁷Br, or ¹⁸F. In some embodiments, the imaging agent or radiotherapeutic agent is administered to a patient and imaged with a photoscanning device or used for the treatment of disease, typically cancer. In some embodiments, the imaging agent or radiotherapeutic agent is administered to a patient and imaged with a photoscanning device or used for the treatment of inflammation. In certain embodiments, ¹⁸F, ⁸⁹Zr, or ⁶⁴Cu are used for positron emission tomography (PET) imaging. In certain embodiments, ¹⁶¹Tb, ¹⁷⁷Lu, ²¹¹At, or ⁹⁰Y are used for radiotherapy. Procedures for labeling biological agents with the radioactive isotopes are generally known in the art.

Two proteins (e.g., an antibody and a heterologous moiety) can be cross-linked using any of a number of known chemical cross linkers. Examples of such cross linkers are those that link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

Techniques for conjugating a therapeutic moiety (e.g., any of those discussed in Section VII) to a B7-H3-specific antibody or antigen binding fragment thereof as described herein are well known, see, for example, Arnon et al., Monoclonal Antibodies And Cancer Therapy, Reisfeld et al. (eds.), pp. 243-56 (1985); Hellstrom et al., Controlled Drug Delivery (2nd Ed.), Robinson et al. (eds.), pp. 623-53 (1987); Thorpe, Monoclonal Antibodies '84:Biological And Clinical Applications, Pinchera et al. (eds.), pp. 475-506 (1985); “Analysis, Results, And Future Prospective Of The Therapeutic Use Of Radiolabeled Antibody In Cancer Therapy” In: Monoclonal Antibodies For Cancer Detection And Therapy, (Baldwin et al. eds.), pp. 303-316 (1985), and Thorpe et al., Immunol. Rev. 62:119-158 (1982). Alternatively, an antibody can be conjugated to a second antibody to form an antibody heteroconjugate (e.g., a bispecific antibody) as described in U.S. Pat. No. 4,676,980.

In some embodiments, a radioactive label can be directly conjugated to the amino acid backbone of the antibody. Alternatively, the radioactive label can be included as part of a larger molecule (e.g., ¹²⁵I in meta-[¹²⁵I]iodophenyl-N-hydroxysuccinimide ([¹²⁵I]mIPNHS), which binds to free amino groups to form meta-iodophenyl (mIP) derivatives of relevant proteins (see, e.g., Rogers et al. (1997) J Nucl Med 38:1221-1229) or chelate (e.g., to DOTA or DTPA), which is in turn bound to the protein backbone. Methods of conjugating the radioactive labels or larger molecules/chelates containing them to the antibodies or antigen binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a fluorophore) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein or fragment thereof with the fluorophore under conditions that facilitate binding of the fluorophore to the protein. See, e.g., Welch and Redvanly (2003) Handbook of Radiopharmaceuticals: Radiochemistry and Applications, John Wiley and Sons.

In some embodiments, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476, or HESylated (Fresenius Kabi, Germany) (see, e.g., Pavisić et al. (2010) Int J Pharm 387(1-2):110-119). The stabilization moiety can improve the stability, or retention of, the antibody (or fragment) by at least 1.5 (e.g., at least 2, 5, 10, 15, 20, 25, 30, 40, or 50 or more) fold.

The present disclosure also includes isotype modification of the antibodies described herein. By modifying the Fc region to have a different isotype, different functionalities can be achieved. For example, changing to IgG1 can increase antibody dependent cell cytotoxicity, switching to class A can improve tissue distribution, and switching to class M can improve valency.

In some embodiments, the antibodies or fragments thereof can be modified to include an Fc region with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be mutated or combined with a binding domain (e.g., an antibody variable domain) and can be assessed using various assays (e.g., Fc binding assays, ADCC assays, CDC assays, etc.).

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J 10(10):2717-2723; and Co et al. (1993) Mol Immunol 30:1361.

### IV. Chimeric Antigen Receptors

Also provided herein are chimeric antigen receptors comprising any of the antibodies or antigen-binding fragments described above. Chimeric antigen receptors (CARs, also known as chimeric T cell receptors) are designed to be expressed in host effector cells, e.g., T cells or NK cells, and to induce an immune response against a specific target antigen and cells expressing that antigen. Adoptive T cell immunotherapy, in which a patient's own T lymphocytes are engineered to express CARs, has shown great promise in treating hematological malignancies. CARs can be engineered and used as described, for example, in Sadelain et al., 2013, Cancer Discov. 3:388-398. A CAR typically comprises an extracellular target-binding module, a transmembrane (TM) domain, and an intracellular signaling domain (ICD). The CAR domains can be joined via flexible hinge and/or spacer regions. The extracellular target-binding module generally comprises an antibody or antigen binding fragment thereof. In some instances, multiple binding specificities can be included in the extracellular target-binding module. For example, multiple antibodies or antigen binding fragments thereof that target different antigens can be included to produce bi-specific, tri-specific, or quad-specific CARs. In some embodiments, the CAR antigen binding domain comprises a heavy chain variable region (VH) having a VHCDR1 amino acid sequence comprising SEQ ID NO: 9, 15, or 21, a VHCDR2 amino acid sequence comprising SEQ ID NO. 10, 16, or 22, and a VHCDR3 amino acid sequence comprising SEQ ID NO. 11, 17, or 23; and a light chain variable region (VL) having a VLCDR1 amino acid sequence comprising SEQ ID NO. 12, 18, or 24, a VLCDR2 amino acid sequence comprising SEQ ID NO. 13, 19 or 25, and a VLCDR3 amino acid sequence comprising SEQ ID NO. 14, 20, or 26, and having an arginine corresponding to position 45 and a tyrosine corresponding to position 70 of SEQ ID NO: 4. TM domains are primarily considered a structural requirement, anchoring the CAR in the cell membrane, and are most commonly derived from molecules regulating T cell function, such as CD8 and CD28. The intracellular module typically consists of the T cell receptor CD3ζ chain and one or more costimulatory domains from either the Ig (CD28-like) or TNF receptor (TNFR) superfamilies. CARs containing either CD28 or 4-1BB costimulatory domains have been the most widely used, to date, and both of them have yielded dramatic responses in clinical trials. CAR domains are discussed in more detail below.

Provided herein are chimeric antigen receptors comprising: (a) an extracellular target-binding domain comprising a B7-H3-specific antibody or antigen binding portion thereof; (b) a transmembrane domain; and (c) a signaling domain.

The extracellular target-binding module of a CAR may comprise an antibody or an antigen-binding fragment thereof that specifically binds a target antigen (e.g., B7-H3). In certain embodiments, the extracellular target-binding domain can be a single-chain variable fragment derived from an antibody (scFv), a tandem scFv, a single-domain antibody fragment (V_{H}Hs or sdAbs), a single domain bispecific antibody (BsAbs), an intrabody, a nanobody, an immunokine in a single chain format, and Fab, Fab′, or (Fab′)₂ in a single chain format. In other embodiments, the extracellular target-binding domain can be an antibody moiety that comprises covalently bound multiple chains of variable fragments. In some embodiments, the extracellular target-binding domain comprises any of the antibodies or antigen-binding portions thereof described above. In some embodiments, the extracellular target-binding domain comprises a scFv comprising a heavy chain variable region comprising an amino acid sequence that is at least 90% identical (for example, at least 90%, 91%, 92%, 93%, 94%, 95%, 96%, 97%, 98% or 99% identical) to any of SEQ ID NOs: 1-3 and a light chain variable region comprising an amino acid sequence that is at least 90% identical (for example, at least 90%, 91%, 92%, 93%, 94%, 95%, 96%, 97%, 98% or 99% identical) to SEQ ID NO: 4. In some embodiments, the extracellular target-binding domain comprises a scFv comprising a heavy chain variable region (VH) having at least 90% identity to SEQ ID NO: 3 and comprising a VHCDR1 amino acid sequence comprising SEQ ID NO: 9, 15, or 21, a VHCDR2 amino acid sequence comprising SEQ ID NO: 10, 16, or 22, and a VHCDR3 amino acid sequence comprising SEQ ID NO: 11, 17, or 23 and comprising a tryptophan at position 47, a methionine at position 48, a valine at position 68, and an arginine at position 72 of SEQ ID NO: 3; and a light chain variable region (VL) having at least 90% identity to SEQ ID NO: 4 and comprising a VLCDR1 amino acid sequence comprising SEQ ID NO: 12, 18, or 24, a VLCDR2 amino acid sequence comprising SEQ ID NO: 13, 19 or 25, and a VLCDR3 amino acid sequence comprising SEQ ID NO: 14, 20, or 26 and comprising an arginine at position 45 and a tyrosine at position 70. In some embodiments, the scFv comprises a linker polypeptide between the heavy chain and light chain sequences (e.g., SEQ ID NO: 43 or SEQ ID NO: 44 or any of the other linkers described herein). In some embodiments, the CAR comprises a scFv comprising an amino acid sequence that is at least 90% identical to SEQ ID NO: 42.

In some embodiments, the extracellular target-binding domains of the CARs provided herein further comprise one or more additional antigen-binding domains (i.e., in addition to the B7-H3-specific antibody or antigen binding portion thereof, as described above). In some embodiments, the extracellular target-binding domain comprises one additional antigen-binding domain. CARs comprising such an extracellular target-binding domain can be referred to as bi-specific CARs. In some embodiments, the extracellular target-binding domain comprises two additional antigen-binding domains. CARs comprising such an extracellular target-binding domain can be referred to as tri-specific CARs. In some embodiments, the extracellular target-binding domain comprises three additional antigen-binding domain. CARs comprising such an extracellular target-binding domain can be referred to as quad-specific CARs. Each of the one or more additional antigen-binding domains may comprise an antibody or antigen binding portion thereof. In some embodiments, the one or more additional antigen-binding domains specifically bind to CD19, CD20, CD22, CD79a, CD79b, or any combination thereof.

The transmembrane domain of a CAR provided herein may be derived from either a natural or from a synthetic source. Where the source is natural, the domain may be derived from any membrane-bound or transmembrane protein. In some embodiments, the transmembrane domain is derived from (i.e., comprises at least the transmembrane region(s) of) the α, β, δ, γ, or ζ chain of the T-cell receptor, CD28, CD3ε, CD3ζ, CD45, CD4, CD5, CD8, CD9, CD16, CD22, CD30, CD33, CD37, CD64, CD80, CD86, CD134, CD137, or CD154. In some embodiments, a transmembrane domain can be chosen based on, for example, the nature of the various other proteins or trans-elements that bind the transmembrane domain or the cytokines induced by the transmembrane domain. In some embodiments, the transmembrane domain comprises a transmembrane domain (e.g., a CD8α transmembrane domain). When a transmembrane domain is synthetic, it may comprise predominantly hydrophobic residues such as leucine and valine. In some embodiments, a triplet of phenylalanine, tryptophan, and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain of a CAR described herein. In some embodiments, the linker is a glycine-serine doublet.

The intracellular signaling domain of the CAR is responsible for activation of at least one of the normal effector functions of the immune cell in which the CAR has been placed in or is designed to be placed in. An effector function of a T cell may be, for example, cytolytic activity or helper activity, including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CARs provided herein include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or costimulatory signal is also required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or costimulatory signal (costimulatory signaling sequences).

Primary signaling sequences regulate primary activation of the TCR complex either in a stimulatory way, or in an inhibitory way. Primary signaling sequences that act in a stimulatory manner may contain signaling motifs which are known as immunoreceptor tyrosine-based activation motifs or ITAMs. In some embodiments, the CARs described herein comprise one or more ITAMs.

Examples of ITAM containing primary signaling sequences that are of particular use in the invention include those derived from TCRζ, FcRγ, FcRβ, CD3γ, CD3δ, CD3ε, CD3ζ, CD5, CD22, CD79a, CD79b, and CD66d. In some embodiments, an ITAM containing primary signaling sequence is derived from CD3ζ.

In some embodiments, the CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR of the invention. In some embodiments, the intracellular signaling domain of a CAR provided herein comprises a CD3ζ primary intracellular signaling sequence and a 4-1BB costimulatory signaling sequence (e.g., the amino acid sequence of SEQ ID NO: 41).

The CARs provided herein may include additional elements, such a signal peptide to ensure proper export of the fusion protein to the cells surface, a leader sequence, and a hinge domain that imparts flexibility to the recognition region and allows strong binding to the targeted moiety. In some embodiments, a spacer domain may be present between any of the domains of the CAR. The spacer domain can be any polypeptide that functions to link two parts of the CAR. A spacer domain may comprise up to about 300 amino acids, including for example about 10 to about 100, or about 25 to about 50 amino acids. Methods of identifying and selecting suitable spacer domains are known.

### V. Antibody Expression and Purification, Nucleic Acids, Vectors, and Cells

The B7-H3 antibodies and antigen binding fragments thereof and molecules comprising such antibodies and antigen binding fragments thereof discussed above (e.g., CARs) may be produced by recombinant expression in a human or non-human cell. Antibody-producing cells include non-human cells expressing heavy chains, light chains, or both heavy and light chains; human cells that are not immune cells expressing heavy chains, light chains, or both heavy and light chains; and human B cells that produce heavy chains or light chains, but not both heavy and light chains. The antibodies and antigen binding fragments thereof of this disclosure may be heterologously expressed, in vitro or in vivo, in cells other than human B cells, such as non-human cells and human cells other than B cells, optionally other than immune cells, and optionally in cells other than cells in a B cell lineage.

The B7-H3 antibodies and antigen binding fragments thereof and molecules comprising them described herein can be produced using a variety of techniques known in the art of molecular biology and protein chemistry. For example, a nucleic acid encoding the antibody or antigen binding fragment thereof can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan & Berg (1981) Proc. Natl. Acad. Sci. USA 78:2072) or Tn5 neo (Southern and Berg (1982)Mol. Appl. Genet. 1:327). The selectable marker gene can be either linked to the DNA gene sequences to be expressed or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc. Natl. Acad. Sci. USA, 79:7147), CMV, polyoma virus (Deans et al. (1984) Proc. Natl. Acad. Sci. USA 81:1292), or SV40 virus (Lusky & Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection.

Appropriate host cells for the expression of antibodies or antigen binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines and hamster cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr. Opin. Biotechnol. 13(6):625-629; van Kuik-Romeijn et al. (2000) Transgenic Res. 9(2):155-59; and Pollock et al. (1999) J. Immunol. Methods 231(1-2):147-57.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression vary with the choice of the expression vector and the host cell and are easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are known in the art (see Ausubel et al. (1988) Current Protocols in Molecular Biology, Wiley & Sons; and Green and Sambrook (2012) Molecular Cloning-A Laboratory Manual, 4th Ed., Cold Spring Harbor Laboratory Press, New York (2001)). The choice of codons, suitable expression vectors and suitable host cells vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220). Additional discussion of expression vectors for use in eukaryotic cells (e.g., for treating a subject with cancer or inflammation), along with suitable delivery systems, is provided in Section VIII.A below.

Also provided herein are nucleic acid molecules encoding a B7-H3 antibody or antigen binding portion thereof that binds specifically to B7-H3 as described in this disclosure. In some embodiments, the nucleic acid encodes a chimeric or grafted antibody as disclosed herein.

In some embodiments, provided are nucleic acid molecules encoding a B7-H3 antibody or antigen binding fragment thereof comprising a heavy chain variable comprising an amino acid sequence that is at least 90% identical (for example, at least 90%, 91%, 92%, 93%, 94%, 95%, 96%, 97%, 98% or 99% identical) to any of SEQ ID NOs: 1-3 and a light chain variable region comprising an amino acid sequence that is at least 90% identical to SEQ ID NO: 4. In some embodiments, the nucleic acid encodes an isolated antibody or antibody fragment comprising a heavy chain variable region (VH) having at least 90% identity to SEQ ID NO: 3 and comprising a VHCDR1 amino acid sequence comprising SEQ ID NO: 9, 15, or 21, a VHCDR2 amino acid sequence comprising SEQ ID NO: 10, 16, or 22, and a VHCDR3 amino acid sequence comprising SEQ ID NO: 11, 17, or 23, and comprising a tryptophan corresponding to position 47, a methionine corresponding to position 48, a valine corresponding to position 68, and an arginine corresponding to position 72 of SEQ ID NO: 3; and a light chain variable region (VL) having at least 90% identity to SEQ ID NO: 4 and comprising a VLCDR1 amino acid sequence comprising SEQ ID NO: 12, 18, or 24, a VLCDR2 amino acid sequence comprising SEQ ID NO: 13, 19 or 25, and a VLCDR3 amino acid sequence comprising SEQ ID NO: 14, 20, or 26 and having an arginine corresponding to position 45 and a tyrosine corresponding to position 70 of SEQ ID NO: 4. In some embodiments, provided are nucleic acid molecules encoding a B7-H3 antibody or antigen binding fragment thereof comprising a heavy chain variable comprising an amino acid sequence that is at least 90% identical to any of SEQ ID NOs: 1-3. In some embodiments, the nucleic acid encodes a humanized monoclonal antibody or antibody fragment comprising a heavy chain variable region (VH) having at least 90% identity to any of SEQ ID NOs: 1-3 and comprising a tryptophan corresponding to position 47, a methionine corresponding to position 48, a valine corresponding to position 68, and an arginine corresponding to position 72 of SEQ ID NO: 3. In some embodiments, provided are nucleic acid molecules encoding a B7-H3 antibody or antigen binding fragment thereof comprising a light chain variable region comprising an amino acid sequence that is at least 90% identical to SEQ ID NO: 4. In some embodiments, the nucleic acid encodes a humanized monoclonal antibody or antibody fragment comprising a light chain variable region (VL) having at least 90% identity to SEQ ID NO: 4 and comprising an arginine corresponding to position 45 and a tyrosine corresponding to position 70 of SEQ ID NO: 4. In some embodiments, provided are nucleic acid molecules encoding a B7-H3 antibody or antigen binding fragment thereof comprising one or more heavy chain CDRs comprising an amino acid sequence that is at least 90% identical to any one of SEQ ID NOs: 9-11, 15-17, and 21-23 and comprising a tryptophan corresponding to position 47, a methionine corresponding to position 48, a valine corresponding to position 68, and an arginine corresponding to position 72 of SEQ ID NO: 3. In some embodiments, provided are nucleic acid molecules encoding a B7-H3 antibody or antigen binding fragment thereof comprising one or more light chain CDRs comprising an amino acid sequence that is at least 90% identical to any one of SEQ ID NOs: 12-14, 18-20, and 24-26 and comprising an corresponding to position 45 and a tyrosine corresponding to position 70 of SEQ ID NO. 4.

In some embodiments, provided are nucleic acid molecules encoding antibodies or antigen binding fragments thereof that bind specifically to B7-H3, wherein the nucleic acid sequences comprise sequences encoding an amino acid sequence that is at least 90% identical (for example, at least 90%, 91%, 92%, 93%, 94%, 95%, 96%, 97%, 98% or 99% identical) to any of the sequences in Table 1. In some embodiments, the nucleic acid molecules comprise sequences encoding an amino acid sequence that is at least 90% identical (for example, at least 90%, 91%, 92%, 93%, 94%, 95%, 96%, 97%, 98% or 99% identical) to any of the sequences in Table 1 and that comprises a heavy chain variable region having a tryptophan corresponding to position 47, a methionine corresponding to position 48, a valine corresponding to position 68, and an arginine corresponding to position 72 of SEQ ID NO: 3, and a light chain variable region (VL) having an arginine corresponding to position 45 and a tyrosine corresponding to position 70 of SEQ ID NO: 4.

In some embodiments, provided are nucleic acid molecules comprising a nucleotide sequence that is at least 90% identical (for example, at least 90%, 91%, 92%, 93%, 94%, 95%, 96%, 97%, 98% or 99% identical) to any of SEQ ID NOs: 27-30. In some embodiments, the nucleic acid molecules comprise sequences that are at least 90% identical (for example, at least 90%, 91%, 92%, 93%, 94%, 95%, 96%, 97%, 98% or 99% identical) to any of SEQ ID NOs: 27-30 and that encode an antibody or antibody fragment that comprises a heavy chain variable region having a tryptophan corresponding to position 47, a methionine corresponding to position 48, a valine corresponding to position 68, and an arginine corresponding to position 72 of SEQ ID NO: 3; and a light chain variable region having an arginine corresponding to position 45 and a tyrosine corresponding to position 70 of SEQ ID NO: 4.

In some embodiments, the nucleic acid molecules encoding the B7-H3 antibodies or antigen binding fragments thereof are synthetic sequences designed for expression in a host cell (for example, a human cell). In some embodiments, the nucleic acid molecule comprises a nucleotide sequence that is at least 90% identical to SEQ ID NOs: 27-30. In some embodiments, the nucleic acid molecules are at least 90% identical (for example, at least 90%, 91%, 92%, 93%, 94%, 95%, 96%, 97%, 98% or 99% identical) to any of SEQ ID NOs: 27-30 and that encode an antibody or antibody fragment that comprises a heavy chain variable region having a tryptophan corresponding to position 47, a methionine corresponding to position 48, a valine corresponding to position 68, and an arginine corresponding to position 72 of SEQ ID NO: 3; and a light chain variable region having an arginine corresponding to position 45 and a tyrosine corresponding to position 70 of SEQ ID NO: 4.

In some embodiments, the nucleic acid molecules encoding the B7-H3 antibodies or antigen binding fragments thereof are operably linked to a promoter capable of directing expression in a bacterial cell or a eukaryotic cell.

The amino acid sequences of the CDRs and framework regions can be determined using various well-known definitions in the art, e.g., Kabat, Chothia, international ImMunoGeneTics database (IMGT), AbM, and observed antigen contacts (“Contact”). In some embodiments, CDRs are determined according to the IMGT definition. See, Brochet et al., 2008, Nucl. Acids Rex. 36:W503-508. In some embodiments, CDRs are determined by a combination of Kabat, Chothia, and/or Contact CDR definitions.

Also provided herein are DNA constructs comprising a promoter that drives expression in a host cell operably linked to a recombinant nucleic acid molecule comprising a nucleotide sequence that encodes a B7-H3 specific antibody or antigen binding fragment thereof.

Also provided herein are vectors, comprising a DNA construct comprising a promoter that drives expression in a host cell operably linked to a recombinant nucleic acid molecule comprising a nucleotide sequence that encodes a B7-H3 specific antibody or antigen binding fragment thereof.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and most preferably cytomegalovirus (CMV), or from heterologous mammalian promoters (e.g., β-actin promoter or EF1α promoter), or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g., chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the beta-actin promoter, the EF1A promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, CT) sequences typically are expressed as a fusion with the encoded polypeptide. Such tags can be inserted anywhere within the polypeptide including at either the carboxyl or amino terminus.

Also provided herein are host cells, including bacterial host cells and eukaryotic host cells, comprising a recombinant nucleic acid molecule encoding a B7-H3 antibody or antigen binding fragment thereof as described in this disclosure. In some embodiments, the nucleic acid molecule comprises a nucleotide sequence that is at least 90% identical to any of SEQ ID NOs: 27-30. In some embodiments, the nucleic acid molecules are at least 90% identical (for example, at least 90%, 91%, 92%, 93%, 94%, 95%, 96%, 97%, 98% or 99% identical) to any of SEQ ID NOs: 27-30 and encode an antibody or antibody fragment that comprises a heavy chain variable region having a tryptophan corresponding to position 47, a methionine corresponding to position 48, a valine corresponding to position 68, and an arginine corresponding to position 72 of SEQ ID NO: 3; and a light chain variable region having an arginine corresponding to position 45 and a tyrosine corresponding to position 70 of SEQ ID NO: 4.

Also provided herein are host cells that have been engineered to express and secrete a B7-H3 antibody or antigen binding fragment thereof as described in this disclosure. In some embodiments, the cells are suitable for implanting in a patient with cancer. In some embodiments, the cells are suitable for implanting in a patient with inflammation. In some embodiments, the cells are animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by a subject's immune system or by other detrimental factors from the surrounding tissues.

Also provided herein are immune cells (e.g., T cells) expressing any of the CARs described herein. In some embodiments, the immune cell expresses the CAR on its surface. In some embodiments, the immune cell comprises a nucleic acid encoding the CAR, wherein the CAR is expressed from the nucleic acid and localized to the immune cell surface. In some embodiments, the immune cell a B-lymphocyte, T-lymphocyte, thymocyte, dendritic cell, natural killer (NK) cell, monocyte, macrophage, granulocyte, eosinophil, basophil, neutrophil, myelomonocytic cell, megakaryocyte, peripheral blood mononuclear cell, myeloid progenitor cell, or a hematopoietic stem cell. In some embodiments, the immune cell is a T cell. In some embodiments, the T cell is a cytotoxic T cell, a helper T cell, a natural killer T cell, a suppressor T cell, a CD8⁺ T cell, a CD4⁺ T cell, a CD8⁺/CD4⁺ T cell, γδ T cell, or a T-regulatory (T-reg) cell.

In some embodiments, immune cells expressing a CAR provided herein are obtained from a subject. Where the immune cells are used to treat (e.g., according to the treatment methods described herein below) the same subject from which they are obtained, they are referred to as autologous cells. Where they are obtained from a different subject, they are referred to as heterologous cells. Immune cells can be isolated from peripheral blood using techniques well known in the art, include Ficoll density gradient centrifugation followed by negative selection to remove undesired cells. In some embodiments, heterologous immune cells useful for the methods provided herein comprise allogeneic T cells, as described in, e.g., Bedoya et al., 2021, Front. Immunol. 12:640082.

In vitro methods are also suitable for preparing monovalent antibodies or antigen binding fragments thereof. Digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen.

The Fab fragments produced in antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing proteins comprising the provided antibodies or fragments is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides, or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

Following expression, the antibodies and fragments thereof can be isolated. An antibody or fragment thereof can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed antibody or fragments thereof is necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### VI. Pharmaceutical Compositions and Formulations

The B7-H3 antibodies and antigen binding portions thereof described herein, as well as the various molecules comprising said antibodies and antigen binding portions thereof (e.g., CARs) are suitable for administration in vitro or in vivo. Compositions comprising a B7-H3 antibody or antigen binding fragment thereof of the present disclosure and a pharmaceutically acceptable carrier (excipient) are provided. In some embodiments, the compositions comprise a CAR comprising the B7-H3 antibody or antigen binding fragment thereof. A pharmaceutically acceptable carrier (excipient) is a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject. The compositions may further comprise a diluent, solubilizer, emulsifier, preservative, and/or adjuvant to be used with the methods disclosed herein. Such compositions can be used, for example, in a subject with cancer or inflammation that would benefit from any of the B7-H3 antibodies or antigen binding fragments thereof described herein.

Suitable carriers and their formulations are described in Remington: The Science and Practice of Pharmacy, 21^{st} Edition, Philip P. Gerbino, ed., Lippincott Williams & Wilkins (2006). In certain embodiments, acceptable formulation materials preferably are nontoxic to recipients at the dosages and concentrations employed. In certain embodiments, the formulation material(s) are for subcutaneous and/or intravenous administration. In certain embodiments, the formulation comprises an appropriate amount of a pharmaceutically-acceptable salt to render the formulation isotonic. In certain embodiments, the pharmaceutical composition can contain formulation materials for modifying, maintaining, or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. In certain embodiments, the optimal pharmaceutical composition is determined by one skilled in the art depending upon, for example, the intended route of administration, delivery format and desired dosage. See, for example, Remington: The Science and Practice of Pharmacy, 22^{nd} Edition, Lloyd V. Allen, Jr., ed., The Pharmaceutical Press (2014). In certain embodiments, such compositions may influence the physical state, stability, rate of in vivo release and/or rate of in vivo clearance of the B7-H3-specific antibody or antigen binding fragment thereof.

In certain embodiments, the primary vehicle or carrier in a pharmaceutical composition can be either aqueous or non-aqueous in nature. For example, in certain embodiments, a suitable vehicle or carrier can be sterile water for injection, physiological saline solution, buffered solutions like Ringer's solution, dextrose solution, or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising a B7-H3-specific antibody or antigen binding fragment thereof disclosed herein can be prepared for storage by mixing the selected composition having the desired degree of purity with optional formulation agents (see Remington: The Science and Practice of Pharmacy, 22^{nd} Edition, Lloyd V. Allen, Jr., ed., The Pharmaceutical Press (2014)) in the form of a lyophilized cake or an aqueous solution. Further, in certain embodiments, a composition comprising a B7-H3-specific antibody or antigen binding fragment thereof disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parenteral delivery (e.g., through injection by intravenous, intraperitoneal, intracerebral (intra-parenchymal), intracerebral, intraventricular, intramuscular, subcutaneous, intra-ocular, intraarterial, intraportal, or intralesional routes). Preparations for parenteral administration can be in the form of a pyrogen-free, parenterally acceptable aqueous solution (i.e., water, alcoholic/aqueous solutions, emulsions, or suspensions, including saline and buffered media) comprising a B7-H3-specific antibody or antigen binding fragment thereof in a pharmaceutically acceptable vehicle. Preparations for parenteral administration can also include non-aqueous solutions, suspensions, and emulsions. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, the compositions can be selected for inhalation or for delivery through the digestive tract, such as orally. Compositions for oral administration include powders or granules, suspension or solutions in water or non-aqueous media, capsules, sachets, or tables. Thickeners, flavorings, diluents, emulsifiers, dispersing aids or binders are optionally desirable.

In certain embodiments, the compositions can be selected for topical delivery. Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8. 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. In some instances, the pH of the pharmaceutical composition may be in the range of 4.0-5.5 such as, for example, 4.0-4.3, 4.0-4.5, 4.2-4.8, 4.5-4.8, 4.5-5.0, 4.8-5.2, or 5.0-5.5.

In certain embodiments, a pharmaceutical composition can comprise an effective amount of a B7-H3 antibody or antigen binding fragment thereof in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving a B7-H3-specific antibody or antigen binding fragment thereof in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; U.S. Pat. Nos. 8,383,153; 4,767,628; International Application Publication No. WO1998043615, Calo et al. (2015) Eur. Polymer J 65:252-267 and European Patent No. EP 058,481), including, for example, chemically synthesized polymers, starch based polymers, and polyhydroxyalkanoates (PHAs), copolymers of L-glutamic acid and gamma ethyl-L-glutamate (Sidman et al. (1993) Biopolymers 22:547-556), poly (2-hydroxyethyl-methacrylate) (Langer et al. (1981) J Biomed Mater Res. 15: 167-277; and Langer (1982) Chem Tech 12:98-105), ethylene vinyl acetate (Hsu & Langer (1985) J Biomed Materials Res 19(4):445-460) or poly-D(−)-3-hydroxybutyric acid (European Patent No. EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692; European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject. In certain examples, the B7-H3-specific antibodies or antigen-binding fragments thereof can be administered at a dose of 1 mg/kg, 2 mg/kg, 3 mg/kg, 4 mg/kg, or 5 mg/kg once every other day at least four times. An exemplary treatment regime may include administration once per day, once per week, twice a week, once every two weeks, once every three weeks, once every four weeks, once a month, once every 3 months, or once every three to 6 months. In some cases, the treatment comprises administering B7-H3-specific antibodies or antigen-binding fragments thereof according to one of the aforementioned dosing regimens for a first period and another of the aforementioned dosing regimens for a second period. In some cases, the treatment discontinues for a period of time before the same or a different dosing regimen resumes. For example, a patient may be on a B7-H3-specific antibody dosing regimen for two weeks, off for a week, on for another two weeks, and so on. Dosage regimens for B7-H3-specific antibodies or antigen-binding fragments thereof of this disclosure include 0.1 mg/kg body weight, 0.3 mg/kg body weight, 2 mg/kg body weight, 3 mg/kg body weight, or 10 mg/kg via intravenous administration, with the B7-H3-specific antibodies or antigen-binding fragments thereof being given using one of the following dosing schedules: (i) every four weeks for six dosages, then every three months; (ii) every three weeks; (iii) 3 mg/kg body weight once followed by 1 mg/kg body weight every three weeks.

In still another aspect, unit dose forms comprising a B7-H3-specific antibody or antigen binding fragment thereof as described in this disclosure are provided. A unit dose form can be formulated for administration according to any of the routes described in this disclosure. In one example, the unit dose form is formulated for intravenous or intraperitoneal administration. In still another aspect, pharmaceutical packages comprising unit dose forms of a B7-H3-specific antibody or antigen binding fragment thereof are provided.

In some instances, the B7-H3 antibody or antigen-binding fragment may be an isolated B7-H3 antibody or antigen-binding fragment thereof as described in this disclosure. The term “isolated,” as used with reference to a protein (or nucleic acid), denotes that the protein (or nucleic acid) is essentially free of other cellular components with which it is associated in the natural state. It is preferably in a homogeneous state. Purity and homogeneity are typically determined using analytical chemistry techniques such as electrophoresis (e.g., polyacrylamide gel electrophoresis) or chromatography (e.g., high performance liquid chromatography). In some embodiments, an isolated protein (or nucleic acid) is at least 85% pure, at least 90% pure, at least 95% pure, or at least 99% pure.

In some instances, the B7-H3 antibody or antigen-binding fragment thereof may be a formulated into virus-like particles (VLPs). VLPs comprise viral protein(s) derived from the structural proteins of a virus. Methods for making and using virus like particles are described in, for example, Garcea and Gissmann, Current Opinion in Biotechnology 15:513-7 (2004).

In some instances, the B7-H3 antibody or antigen-binding fragment thereof may be a formulated into subviral dense bodies (DBs). DBs transport proteins into target cells by membrane fusion. Methods for making and using DBs are described in, for example, Pepperl-Klindworth et al., Gene Therapy 10:278-84 (2003).

### VI. Kits and Packaging

The B7-H3 antibodies and antigen binding fragments thereof disclosed herein may be used for the preparation of a kit (e.g., a diagnostic test kit, a radioligand therapy kit, or kit for the treatment of a patient). In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the method.

In some embodiments, one of the containers may comprise a B7-H3 antibody or antigen binding fragment thereof as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. For example, a kit for imaging a tumor in a subject with a B7-H3 expressing cancer is provided herein. In another example, a kit for radioligand therapy of a tumor in a subject with a B7-H3 expressing cancer is provided herein. In some embodiments, a kit for imaging a subject with inflammation expressing B7-H3 is provided herein. In some embodiments, the kit comprises a container containing a labeled B7-H3 antibody or antigen binding fragment thereof. In some embodiments, the kit comprises separate containers containing a B7-H3 antibody or antigen binding fragment thereof and a detectable label or a radioisotope.

A B7-H3 antibody or antigen binding fragment thereof as described in this disclosure for use in treating cancer patients may be delivered in a pharmaceutical package or kit to doctors, healthcare providers, treatment facilities, or cancer patients. Such packaging is intended to improve patient convenience and compliance with the treatment plan. Typically, the packaging comprises paper (cardboard) or plastic. In some embodiments, the kit or pharmaceutical package further comprises instructions for use (e.g., for administering according to a method as described herein).

In some embodiments, a B7-H3 antibody or antigen binding fragment thereof as described in this disclosure for use in treating patients with inflammation may be delivered in a pharmaceutical package or kit to doctors, healthcare provides, treatment facilities, or patients with inflammation (e.g., to improve patient convenience and compliance with the treatment plan). Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of a B7-H3 antibody or antigen binding fragment. In some embodiments, the pharmaceutical package or kit further comprises unit dose forms of one or more of a chemotherapeutic agent, a cytotoxic agent, a radiotherapeutic agent, or an immunotherapeutic agent.

In one embodiment, the kit or pharmaceutical package comprises a B7-H3 antibody or antigen binding fragment in a defined, therapeutically effective dose in a single unit dosage form or as separate unit doses. The dose and form of the unit dose (e.g., pre-filled syringe, tablet, capsule, immediate release, delayed release, etc.) can be any doses or forms as described herein.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

In some embodiments, the kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a therapeutically effective amount of a therapeutic agent.

### VIII. Methods of Use

Provided herein are methods to treat or inhibit a disease, condition, or disorder associated with elevated levels of B7-H3, such as cancer, such as renal, pancreatic, colorectal, non-small cell lung, ovarian, bladder, melanoma, prostate, and neuroectodermal cancer. Functioning of B7-H3 may be reduced by any suitable drugs. Preferably, such substances would be an anti-B7-H3 antibody or antigen binding fragment thereof as described in this disclosure. The methods comprise administering to a subject a therapeutically effective amount of a composition comprising an isolated B7-H3-specific antibody or antigen binding portion thereof described herein. Also, provided are prognostic and diagnostic methods for cancer based on detection and/or quantitation of B7-H3 using a B7-H3 antibody or antigen binding fragment as described in this disclosure. Also provided are methods of detecting the presence of B7-H3 protein in a sample using the described B7-H3 antibodies or antigen binding fragments.

Provided herein are methods to treat or inhibit an inflammatory disease, condition, or disorder associated with elevated levels of B7-H3. Functioning of B7-H3 may be reduced by any suitable drugs. Preferably, such substances would be an anti-B7-H3 antibody or antigen binding fragment thereof as described in this disclosure. The methods comprise administering to a subject a therapeutically effective amount of a composition comprising an isolated B7-H3-specific antibody or antigen binding portion thereof described herein. Also, provided are prognostic and diagnostic methods for inflammation based on detection and/or quantitation of B7-H3 using a B7-H3 antibody or antigen binding fragment as described in this disclosure. Also provided are methods of detecting the presence of B7-H3 protein in a sample using the described B7-H3 antibodies or antigen binding fragments thereof.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably, and the term patient or subject includes human and veterinary subjects. The B7-H3 antibody or antigen binding portion thereof described herein are useful for treating cancer in humans, including, without limitation, pediatric and geriatric populations, and in animals, e.g., veterinary applications. In one embodiment, the subject is a human. In some embodiments, the B7-H3 antibody or antigen binding portion thereof described herein are useful for treating inflammation in humans, including, without limitation, pediatric and geriatric populations, and in animals, e.g., veterinary applications. In one embodiment, the subject is a human.

As used herein the terms “cancer” and “tumor” are used to indicate malignant tissue. The term, “cancer” is also used to refer to the disease associated with the presence of malignant tumor cells in an individual, and the term “tumor” is used to refer to a plurality of cancer cells that are physically associated with each other. Cancer cells are malignant cells that give rise to cancer, and tumor cells are malignant cells that can form a tumor and thereby give rise to cancer.

As used herein the term “inflammation” is used to indicate a local or whole body response to cellular injury that is marked by capillary dilation, leukocytic infiltration, redness, heat, pain, swelling, and often loss of function and that serves as a mechanism initiating the elimination of noxious agents and of damaged tissues.

### A. Methods of Treatment

Provided herein are methods to treat cancer in a subject using a B7-H3 antibody or antigen binding fragment thereof as described in this disclosure.

In another embodiment provided herein are methods for treating inflammation in a subject using a B7-H3 antibody or antigen binding fragment thereof as described in this disclosure.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

An “inflammatory condition” or “inflammatory disorder” or the like refers to any inflammation in an individual, and can be transient (e.g., in response to exposure to a pathogen or allergen) or chronic. Inflammation is characterized by pro-inflammatory cytokines, such as IFN-gamma, IL-6, and TNF-alpha, that recruit and activate macrophages and other leukocytes. In some cases, inflammation can develop into a chronic, harmful condition or autoimmune condition (e.g., multiple sclerosis, lupus, rheumatoid arthritis, Crohn's disease). Inflammation can be evident locally (e.g., at a localized site of infection or exposure) or systemically (e.g., atherosclerosis, high blood pressure).

As used herein, an “effective amount” means the amount of an agent that is effective for producing a desired effect in a subject. The actual dose that comprises the effective amount may depend upon the route of administration, the size and health of the subject, the disorder being treated (e.g., cancer or inflammation), and the like.

In some embodiments, the B7-H3 antibody or antigen binding fragment thereof can directly inhibit growth and induce cell death of cancer cells. In some instances, the B7-H3 antibody or antigen binding fragment thereof may inhibit tumor initiation, e.g., by binding to B7-H3 expressed by undifferentiated leukemia cells or cancer stem cells. In some instances, the B7-H3 antibody or antigen binding fragment thereof can sensitize cancer cells to other cancer therapies (e.g., chemotherapy). In some instances, treating a subject according to the methods described herein inhibits at least one of formation of a tumor, the proliferation of tumor cells, the growth of tumor cells, survival of tumor cells in circulation, or metastasis of tumor cells in the individual. In another embodiment, treating a subject according to the methods described herein may result in tumor growth stasis, reduction of tumor size and, in some instances, elimination of one or more tumors in the subject.

In some embodiments, the B7-H3 antibody or antigen binding fragment thereof itself may not be therapeutic but may be used to target a therapeutic agent to cancer stem cells, as discussed further herein. In such instances, the B7-H3 antibody or antigen binding fragment thereof need only bind specifically to the B7-H3 protein. Thus, in some embodiments, the B7-H3 antibody or antigen binding fragment thereof may be conjugated to a therapeutic pharmaceutical agent, as described herein.

In some embodiments, the B7-H3 antibody or antigen binding fragment thereof can directly inhibit inflammation in cells. In some instances, the B7-H3 antibody or antigen binding fragment thereof can sensitize inflammation in cells to other anti-inflammation therapies. In some instances, treating a subject according to the methods described herein inhibits inflammation in cells in the individual. In another embodiment, treating a subject according to the methods described herein may result in inflammation stasis, reduction of inflammation and, in some instances, elimination of inflammation in the subject.

In some embodiments, the B7-H3 antibody or antigen binding fragment thereof itself may not be therapeutic but may be used to target a therapeutic agent to inflammation cells, as discussed further herein. In such instances, the B7-H3 antibody or antigen binding fragment thereof need only bind specifically to the B7-H3 protein. Thus, in some embodiments, the B7-H3 antibody or antigen binding fragment thereof may be conjugated to a therapeutic pharmaceutical agent, as described herein.

In some embodiments, the B7-H3 antibody or antigen binding fragment thereof may be conjugated to a radioisotope for use as a radioligand therapeutic. Radioactively labeled monoclonal antibodies and antibody fragments of the present disclosure may be produced according to well-known methods in the art. For example, monoclonal antibodies can be iodinated by contact with sodium and/or potassium iodide and a chemical oxidizing agent such as sodium hypochlorite, or an enzymatic oxidizing agent, such as lactoperoxidase. In some embodiments, the B7-H3 antibody or antigen binding fragment thereof according to this disclosure may be labeled with technetium-99m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl2, a buffer solution such as sodium-potassium phthalate solution, and the antibody. Intermediary functional groups that incorporate chelators, which are often used to bind radioisotopes that exist as metallic ions to an antibody are diethylene-triamine-pentaacetic acid (DTPA), ethylene diamine-tetraacetic acid (EDTA), monomeric or dendrimeric 1,4,7,10-tetraazacyclododecane-1,4,7,10-tetraacetic acid (DOTA), 1,4,7-triazacyclononane-1,4,7-triacetic acid (NOTA), deferoxamine (DFO), or 1-hydroxy-2(1H)-pyridinone derivatives (e.g., 3,4,3-LI(1,2-HOPO) or HOPO).

Also provided are cancer treatment methods using a radioligand therapeutic comprising a B7-H3 antibody or antigen binding fragment thereof as described in this disclosure. Dosage ranges for radioisotopes vary widely, and depend on the half-life of the isotope, the strength and type of radiation emitted, and the uptake by the neoplastic cells. Exemplary dosing regimens can be found in U.S. Pat. Nos. 5,595,721 and 6,015,542, which are each incorporated herein by reference in their entirety. For example, a radiolabeled antibody may be administered in a single dose designed to deliver a high amount of radioactivity. In such a method, it is contemplated that a radiometric dose of greater than 200 cGy is delivered to the whole body of the patient. In this “high-dose” method, bone marrow transplantation, or some other means of reconstituting hematopoietic function in the patient, is required.

A therapeutic dose of radiolabeled antibody may be administered, however, wherein the radiometric dose received by the patient is limited to a level that toxicity to bone marrow is not significant and reconstitution of hematopoietic function, by bone marrow transplantation or other means, is not required. For example, a dose range effective in this method is one which delivers between 25 and 200 cGy, preferably 25 to 150 cGy to the whole body of the patient.

Also provided are cancer treatment methods using a CAR comprising a B7-H3 antibody or antigen binding fragment thereof as described in this disclosure. In some embodiments, these methods comprise using the CAR to redirect the specificity of an immune effector cell (e.g., a T cell) to target a cancer cell (e.g., a B7-H3 expressing cancer cell). Thus, provided herein are methods of stimulating an effector cell-mediated response (such as a T cell-mediated immune response) to a target cell population or tissue comprising cancer cells in a mammal, comprising the step of administering to the mammal an effector cell (such as a T cell) that expresses a CAR as described herein. In some embodiments, “stimulating” an immune cell refers to eliciting an effector cell-mediated response (such as a T cell-mediated immune response), which is different from activating an immune cell. CAR-expressing effector cells described herein can be infused to a subject in need of treatment (e.g., a cancer patient). In some embodiments, the infused cell is able to kill (or lead to the killing of) cancer cells in the subject. Formulations and methods for making CAR-expressing effector cells and using them in therapeutic methods are known in the art (see, e.g., Feins et al., 2019, Am. J. Hematol. 94(S1): S3-S9).

Also provided are treatment methods for inflammation using a CAR comprising a B7-H3 antibody or antigen binding fragment thereof as described in this disclosure. In some embodiments, these methods comprise using the CAR to redirect the specificity of an immune effector cell to target inflammation (e.g., a B7-H3 expressing cell). CAR-expressing effector cells described herein can be infused to a subject in need of treatment (e.g., a patient having or experiencing inflammation). In some embodiments, the infused cell is able to reduce inflammation in the subject. Formulations and methods for making CAR-expressing effector cells and using them in therapeutic methods are known in the art (see, e.g., Feins et al., 2019, Am. J. Hematol. 94(S1): S3-S9).

The subject to be treated by any of the methods herein may have one of various of different cancers, including, for example, lymphoma, follicular lymphoma (FL), mantle cell lymphoma (MCL), diffuse large B-cell lymphoma (DLBCL), leukemia, chronic lymphocytic leukemia (CLL), marginal zone lymphoma, breast cancer, ovarian cancer, colon cancer, lung cancer, skin cancer, pancreatic cancer, testicular cancer, bladder cancer, uterus cancer, prostate cancer, or adrenal cancer. In some instances, the subject may have a primary cancer. In other instances, the subject may have metastatic cancer. In some embodiments, the cancer comprises cells that abnormally express B7-H3 at a level above basal expression in corresponding normal/non-cancer cells (i.e., a B7-H3 expressing cancer). In some embodiments, the subject can have chronic lymphocytic leukemia. In some embodiments, the subject can have mantle cell lymphoma. In some embodiments, the subject can have breast cancer. In some embodiments, the subject can have lung cancer.

The subject to be treated by any of the methods herein may have one of various of different inflammatory conditions or disorders. In some embodiments, the inflammatory condition or disorder can be one selected from the group consisting of systemic lupus erythematosus (SLE), Sjogren's syndrome, dermatitis, Type 1 diabetes, Type 2 diabetes, thyroiditis, Addison disease, pernicious anemia, autoimmune hepatitis, inflammatory bowel disease, multiple sclerosis, encephalitis, rheumatoid arthritis, myasthenia gravis, neuritis, primary biliary cholangitis, Goodpasture's disease, primary membranous nephropathy, cystitis, ovarian insufficiency, autoimmune orchitis, chronic obstructive pulmonary disease (COPD), asthma, pneumonitis, high blood pressure, heart disease, myositis, myocarditis, inflammatory arteritis (Takayasu arteritis, giant cell arteritis), lymphangitis, Parkinson's disease, or graft versus host disease. Other such autoimmune diseases are known in the art (see, e.g., Ludwig et al. (2017, Frontiers in Immunol 8:Article 603; Hofmann et al., 2018, Frontiers in Immunol 9:Article 835). In some embodiments, the inflammatory condition is in response to exposure to a pathogen (e.g., bacteria, virus, fungi), an environmental chemical, or radiation.

In some embodiments, B7-H3 expression (e.g., in cancer cells or inflammation) can be examined by using one or more routine biochemical analyses. In some embodiments, B7-H3 expression is determined by detecting protein expression using methods such as mass spectrometry, western blot analysis, flow cytometry, and immunohistochemistry staining. In some embodiments, such methods comprise use of a B7-H3 antibody or antigen binding portion thereof (e.g., as described in this disclosure). In some embodiments, B7-H3 expression is determined by detecting mRNA levels using methods such as RT-PCR, RNA sequencing, microarray analysis, and northern blot analysis. In some instances, a combination of these methods may be used, or additional methods known in the art may also be used.

In one embodiment, CAR T cells comprising B7-H3 specific antibodies provided herein induce significant lysis of cells expressing human B7-H3 (e.g., B7-H3 expressing L cells and cells from B-cell lymphoma cell lines including Jeko-1, sp53, and CA46), but not cells that do not express B7-H3 (e.g., parental L cells and cells from a B7-H3-negative leukemia cell line NK92).

“Treat,” “treatment,” and the like are used herein to generally mean obtaining a desired pharmacologic and/or physiologic effect. In some embodiments, “treating” or “treatment” may refer to any indicia of success in the treatment or amelioration of cancer. “Treating” or “treatment” includes the administration of an agent to impede growth of a cancer, to do one or more of the following: cause a cancer to shrink by weight or volume, extend the expected survival time of the subject, or extend the expected time to progression of the tumor, or the like. In some embodiments, “treating” or “treatment” may refer to any indicia of success in the treatment or amelioration of inflammation (e.g., reduction of redness, pain, heat). The effect of treatment can be compared to an individual or pool of individuals not receiving the treatment, or to the same patient prior to treatment or at a different time during treatment.

The term “administer,” as used herein, refers to a method of delivering agents, compounds, or compositions to the desired site of biological action. The pharmaceutical compositions (e.g., as described above) are prepared for administration in a number of ways, including but not limited to injection, ingestion, transfusion, implantation, or transplantation, depending on whether local or systemic treatment is desired, and on the area to be treated. The preparation of such pharmaceutically acceptable compositions is within the ability of one skilled in the art. The compositions are administered via any of several routes of administration, including topical, oral, parenteral, intravenous, intra-articular, intraperitoneal, intramuscular, subcutaneous, intracavity, intralesional, transdermal, intradermal, intrahepatical, intrathecal, intracranial, rectal, transmucosal, intestinal, ocular, intra-ocular, otic, nasal, inhalation, or intrabronchial delivery, or any other method known in the art. In some embodiments, the B7-H3 antibody or antigen binding fragment thereof is administered orally, intravenously, or intraperitoneally.

In one aspect, provided is a method of treating a subject with cancer, the method comprising administering to the patient a therapeutically effective amount of a composition comprising a B7-H3 antibody or antigen binding portion thereof as described in this disclosure. The composition may further comprise a pharmaceutically acceptable carrier.

In another aspect, provided is a method of treating a subject with inflammation, the method comprising administering to the patient a therapeutically effective amount of a composition comprising a B7-H3 antibody or antigen binding portion thereof as described in this disclosure. The composition may further comprise a pharmaceutically acceptable carrier.

In some instances, the B7-H3 antibody or antigen-binding fragment thereof can be administered via virus-like particles. Virus-like particles may be formulated as described as above.

In some instances, the B7-H3 antibody or antigen-binding fragment thereof can be administered by subviral dense bodies. Dense bodies may be formulated as described above.

In some instances, the B7-H3 antibody or antigen-binding fragment thereof can be administered by tegument aggregates. Methods for making and using tegument aggregates are described in International Publication No. WO 2006/110728.

In another aspect, provided is a method of treating a subject with cancer, the method comprising administering to the patient cells that have been genetically engineered, using methods such as those described herein, to express and secrete a B7-H3 antibody or antigen binding portion thereof as described in this disclosure.

In another aspect, provided is a method of treating a subject with cancer, the method comprising administering to the patient immune cells that express a CAR comprising a B7-H3 antibody or antigen binding portion thereof as described herein.

In another aspect, provided is a method of treating a subject with cancer, the method comprising administering to the patient a vector comprising a nucleic acid sequence encoding the B7-H3 antibody or antigen binding fragment thereof as described in this disclosure. In some embodiments, the nucleic acid molecule comprises a nucleotide sequence that is at least 90% identical to any one of SEQ ID NOs: 27-30. In some embodiments, the nucleic acid molecules are at least 90% identical (for example, at least 90%, 91%, 92%, 93%, 94%, 95%, 96%, 97%, 98% or 99% identical) to any of SEQ ID NOs: 27-30 and comprise a heavy chain variable region having a tryptophan corresponding to position 47, a methionine corresponding to position 48, a valine corresponding to position 68, and an arginine corresponding to position 72 of SEQ ID NO: 3; and a light chain variable region having an arginine corresponding to position 45 and a tyrosine corresponding to position 70 of SEQ ID NO: 4.

In yet another aspect, provided is a method of treating a subject with inflammation, the method comprising administering to the patient cells that have been genetically engineered, using methods such as those described herein, to express and secrete a B7-H3 antibody or antigen binding portion thereof as described in this disclosure.

In another aspect, provided is a method of treating a subject with inflammation, the method comprising administering to the patient immune cells that express a CAR comprising a B7-H3 antibody or antigen binding portion thereof as described herein.

In another aspect, provided is a method of treating a subject with inflammation, the method comprising administering to the patient a vector comprising a nucleic acid sequence encoding the B7-H3 antibody or antigen binding fragment thereof as described in this disclosure.

In some embodiments, the nucleic acid molecule comprises a nucleotide sequence that is at least 90% identical to any one of SEQ ID NOs: 27-30. In some embodiments, the nucleic acid molecules are at least 90% identical (for example, at least 90%, 91%, 92%, 93%, 94%, 95%, 96%, 97%, 98% or 99% identical) to any of SEQ ID NOs: 27-30 and comprise a heavy chain variable region having a tryptophan corresponding to position 47, a methionine corresponding to position 48, a valine corresponding to position 68, and an arginine corresponding to position 72 of SEQ ID NO: 3; and a light chain variable region having an arginine corresponding to position 45 and a tyrosine corresponding to position 70 of SEQ ID NO: 4.

There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based delivery systems. Such methods are well known in the art and readily adaptable for use with the compositions and methods described herein.

As used herein, plasmid or viral vectors are agents that transport the disclosed nucleic acids into the cell without undesired degradation and include a promoter yielding expression of the nucleic acid molecule and/or adapter polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors. In some instances, the nucleic acid molecules encoding the B7-H3 antibodies or antigen-binding fragments thereof can be delivered via virus-like particles.

Non-viral based delivery methods, can include expression vectors comprising nucleic acid molecules and nucleic acid sequences encoding the adapter polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, WI), Clonetech (Pal Alto, CA), Stratagene (La Jolla, CA), and Invitrogen/Life Technologies (Carlsbad, CA). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

In certain embodiments, the effective amount of a pharmaceutical composition comprising a B7-H3-specific antibody or antigen binding fragment thereof to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a B7-H3-specific antibody or antigen binding fragment thereof is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the B7-H3-specific antibody or antigen binding fragment thereof in the formulation used. Such pharmacokinetic parameters are well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J Clin. Pharmacol. 24:103-108; the latest Remington's, supra). In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data

In certain embodiments, the route of administration of the pharmaceutical composition is in accord with known methods, e.g., orally, through injection by intravenous, intraperitoneal, intracerebral (intra-parenchymal), intracerebral, intraventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of a combination therapy may be administered by different routes.

In certain embodiments, the composition can be administered locally, e.g., during surgery or topically. Optionally local administration is via implantation of a membrane, sponge, or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

In certain embodiments, it can be desirable to use a pharmaceutical composition comprising a B7-H3 antibody or antigen binding fragment thereof in an ex vivo manner. In such instances, cells that have been removed from a subject may be exposed to a pharmaceutical composition comprising a B7-H3 antibody or antigen binding fragment thereof after which the cells are subsequently implanted back into the subject.

In some embodiments, the provided methods may include administering to the subject a B7-H3-specific antibody or antigen binding fragment thereof that is conjugated to a therapeutic agent. The therapeutic agent may be at least one of a cytotoxic agent, a chemotherapeutic agent, a radiotherapeutic agent, a phototherapeutic agent, or an immunosuppressive agent. Such therapeutic agents are described herein.

In some embodiments, the provided methods may include administering a B7-H3-specific antibody or antigen binding fragment thereof and a second form of cancer therapy to the subject. The second form of cancer therapy may include a cytotoxic agent, a chemotherapeutic agent, a radiotherapeutic agent, a phototherapeutic agent, an immunosuppressive agent (including immune checkpoint inhibitors), or radiation therapy. In some embodiments, the second form of cancer therapy is an antibody (e.g., a monoclonal antibody). Monoclonal antibodies which may be administered as a second form of cancer therapy include, but are not limited to, rituximab (e.g., for treatment of B-cell lymphomas), trastuzumab (e.g., for treatment of breast cancer), and cetuximab (e.g., for treatment of lung cancer).

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radioligand therapy, external beam radiotherapy, surgical therapy, immunotherapy, or radioimmunotherapy.

The methods and compositions, including combination therapies, enhance the therapeutic or protective effect, and/or increase the therapeutic effect of another anti-inflammatory therapy. Therapeutic and prophylactic methods and compositions can be provided in a combined amount effective to achieve the desired effect, such as the reduction of inflammation. This process may involve contacting the cells with both an antibody or antibody fragment, or a cell-based therapy expressing an antibody fragment, and a second therapy. A tissue, tumor, or cell can be contacted with one or more compositions or pharmacological formulation(s) comprising one or more of the agents (i.e., antibody or antibody fragment or an anti-inflammatory agent), or by contacting the tissue, tumor, and/or cell with two or more distinct compositions or formulations, wherein one composition provides 1) an antibody or antibody fragment, 2) an anti-inflammatory agent, or 3) both an antibody or antibody fragment and an anti-inflammatory agent. In some embodiments, the anti-inflammatory agent is selected from a group consisting of corticosteroids, DMARDs, or anti-cytokine therapies, or a combination thereof. In some embodiments, the process may involve low doses of radioactive conjugates as described herein.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some instances, the B7-H3 antibody or antigen binding fragment thereof can be labeled, conjugated, or fused with a therapeutic agent or diagnostic agent (such as an imaging agent). The linkage can be covalent or noncovalent (e.g., ionic). Such antibodies and antibody fragments are referred to antibody-drug conjugates (ADC) or immunoconjugates. The antibody conjugates are useful for the local delivery of therapeutic agents, particularly cytotoxic or cytostatic agents, i.e., drugs to kill or inhibit tumor cells in the treatment of cancer, allowing targeted delivery of the drug moiety to tumors, and intracellular accumulation therein, where systemic administration of these unconjugated drug agents may result in unacceptable levels of toxicity to normal cells as well as the tumor cells sought to be eliminated. Therapeutic agents include but are not limited to toxins, including but not limited to plant and bacterial toxins, small molecules, radioligands, bifunctional chelates, peptides, polypeptides, and proteins. Genetically engineered fusion proteins, in which genes encoding for an antibody, or fragments thereof including the Fv region, or peptides can be fused to the genes encoding a toxin to deliver a toxin to the target cell are also provided. As used herein, a target cell or target cells are B7-H3 positive cells.

In some embodiments, the B7-H3 antibody or antigen binding fragment thereof is conjugated to a moiety that specifically binds to an immune cell. In some embodiments, provided is a bispecific antibody comprising a B7-H3 antibody or antigen binding fragment thereof as described herein and an antibody or antigen binding fragment thereof that specifically binds to an immune cell. In some embodiments, the bispecific antibody comprises a B7-H3-specific antibody or antigen-binding portion thereof and an antibody moiety that specifically binds to T cells. Such a molecule is referred to as a bispecific T cell engager and may induce T cell-mediated cytotoxicity of B7-H3 expressing cancer cells (see, e.g., Zhou et al., 2021, Biomarker Research 9:38). In some embodiments, the bispecific antibody comprises a B7-H3-specific antibody or antigen-binding portion thereof and an antibody moiety that specifically binds to natural killer cells (NK cells). Such a molecule is referred to as a NK cell engager and may induce NK cell-mediated cytotoxicity of B7-H3 expressing cancer cells (see, e.g., Demaria et al., 2021, European Journal of Immunology 51(8):1934-1942).

Other examples of therapeutic agents include chemotherapeutic agents, a radiotherapeutic agent, a phototherapeutic agent, and immunotherapeutic agent, as well as combinations thereof. In this way, the antibody or peptide complex delivered to the subject can be multifunctional, in that it exerts one therapeutic effect by binding to the B7-H3 protein and a second therapeutic effect by delivering a supplemental therapeutic agent.

The therapeutic agent can act extracellularly, for example by initiating or affecting an immune response, or it can act intracellularly, either directly by translocating through the cell membrane or indirectly by, for example, affecting transmembrane cell signaling. The therapeutic agent is optionally cleavable from the B7-H3 antibody or antigen binding fragment thereof. Cleavage can be autolytic, accomplished by proteolysis, or affected by contacting the cell with a cleavage agent.

In some instances, the therapeutic agent is a cytotoxic agent. A cytotoxin or cytotoxic agent includes any agent that is detrimental to cells. Examples of toxins or toxin moieties include diphtheria, ricin, streptavidin, and modifications thereof. Additional examples include paclitaxel, cisplatin, carboplatin, cytochalasin B, gramicidin D, ethidium bromide, emetine, etoposide, tenoposide, colchicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof. Therapeutic agents include, but are not limited to, antimetabolites (e.g., methotrexate, 6-mercaptopurine, 6-thioguanine, cytarabine, 5-fluorouracil, decarbazine), alkylating agents (e.g., mechlorethamine, thiotepa, chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e. g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Cytotoxic peptides such as auristatin (antineoplastic) peptides auristatin E (AE) and monomethylauristatin (MMAE), which are synthetic analogs of dolastatin, may also be conjugated to the B7-H3-specific antibody or antigen binding fragment thereof. In some instances, the B7-H3-specific antibody or antigen binding fragment thereof may be conjugated to a radioactive metal ion.

As referred to herein, a chemotherapeutic agent is a chemical compound useful in the treatment of cancer. Examples of chemotherapeutic agents include erlotinib (such as TARCEVA®, Genentech/OSI Pharm.), bortezomib (such as VELCADE®, Millenium Pharm.), fulvestrant (such as FASLODEX®, AstraZeneca), sutent (such as SU11248, Pfizer), letrozole (such as FEMARA®, Novartis), imatinib mesylate (such as GLEEVEC®, Novartis), PTK787/ZK222584 (Novartis), oxaliplatin (such as Eloxatin®, Sanofi), 5-fluorouracil (5-FU), leucovorin, rapamycin (also known as sirolimus) (such as RAPAMUNE®, Wyeth), lapatinib (such as TYKERB®, GSK572016, GlaxoSmithKline), lonafarnib (such as SCH 66336), sorafenib (such as BAY43-9006, Bayer Labs.), capecitabine (such as XELODA®, Roche), docetaxel (such as TAXOTERE®), and gefitinib (such as IRESSA®, Astrazeneca), AG1478, AG1571 (such as SU 5271; Sugen Inc.), alkylating agents such as thiotepa and cyclosphosphamide (such as CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, particularly calicheamicin γ₁¹ and calicheamicin θ₁¹); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (such as ADRIAMYCIN®, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; Trametes versicolor polysaccharide-K (Krestin, PSK) (JHS Natural Products, Eugene, OR); razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; cytarabine (cytosine arabinoside, “Ara-C”); cyclophosphamide; thiotepa; taxoids, e.g., paclitaxel (such as TAXOL®, Bristol-Myers Squibb Oncology, Princeton, N.J.), ABRAXANE™ (a Cremophor-free, albumin-engineered nanoparticle formulation of paclitaxel (American Pharmaceutical Partners, Schaumberg, IL)), and doxetaxel (such as TAXOTERE®, Rhône-Poulenc Rorer, Antony, France); chloranbucil; gemcitabine (such as GEMZAR®); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (such as NAVELBINE®); novantrone; teniposide; edatrexate; daunomycin; aminopterin; xeloda; ibandronate; CPT-11; topoisomerase inhibitor RFS 2000; difluorometlhylomithine (DMFO); retinoids such as retinoic acid; capecitabine; and pharmaceutically acceptable salts, acids or derivatives of any of the above.

Chemotherapeutic agents, as used herein, also refers to (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY 117018, onapristone, and toremifene (such as FARESTON®); (ii) aromatase inhibitors that inhibit the enzyme aromatase, which regulates estrogen production in the adrenal glands, such as, for example, 4(5)-imidazoles, aminoglutethimide, megestrol acetate (such as MEGASE®), exemestane (such as AROMASIN®), formestanie, fadrozole, vorozole (such as RIVISOR®), letrozole (such as FEMARA®), and anastrozole (such as ARIMIDEX®); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) VEGF receptor and angiogenesis inhibitors (including ribozymes such as ANGIOZYME®) and a HER2 expression inhibitor; (ix) vaccines such as gene therapy vaccines, for example, ALLOVECTIN-7@vaccine (plasmid/lipid complex containing the DNA sequences encoding HLA-B7 and 82 microglobulin), LEUVECTIN® vaccine (plasmid DNA expression vector encoding interleukin-2 (IL-2) complexed with a lipid delivery vehicle (DMRIE/DOPE)), and VAXID® vaccine (patient-specific naked DNA vaccine); IL-2 or aldesleukin (such as PROLEUKIN®); topoisomerase 1 inhibitors (such as TOPOTECAN®); gonadotropin-releasing hormone antagonists (such as ABARELIX®); (x) anti-angiogenic agents such as bevacizumab (such as AVASTIN®, Genentech); and (xi) pharmaceutically acceptable salts, acids or derivatives of any of the above.

In some instances, the treatment methods provided herein may further comprise administering an immunosuppressive agent such as an immune checkpoint inhibitor as part of the method. These treatments work by “taking the brakes off” the immune system (are immunosuppressive), allowing it to mount a stronger and more effective attack against cancer. Several different types of checkpoint inhibitors, targeting different checkpoints or “brakes” on immune cells, are currently in use. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumour necrosis factor receptor-related protein (GITR), HLA-DRB 1, ICOS (also known as CD278), HLA-DQAl, HLA-E, indoleamine 2,3-dioxygenase 1 (IDOl), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-Ll, also known as CD274), PDCD1LG2, PSMB 10, ST A T1, T cell immunoreceptor with lg and ITIM domains (TI GIT), T-cell immunoglobulin domain and mucin domain 3 (TIM-3), and V-domain lg suppressor of T cell activation (VISTA, also known as C10orf54). In particular, the immune checkpoint inhibitors target the PD-1 axis and/or CTLA-4. Exemplary immunosuppressive agents are PD-1 inhibitors (such as nivolumab and pembrolizumab), PD-L1 inhibitors (such as atezolizumab, durvalumab, and avelumab), and CTLA-4 inhibitors (such as ipilimumab). In one example, the second form of cancer therapy comprises a PD-L1 inhibitor, a PD-1 inhibitor, or a CTLA4 inhibitor. In some instances, combinations of such inhibitors can be administered. In some instances, the PD-L1 inhibitor, the PD-1 inhibitor, and/or the CTLA4 inhibitor may be an inhibitory antibody that binds specifically to PD-L1, PD-1, or CTLA4, respectively.

In some instances, the treatment methods provided herein may further comprise administering radiation therapy to the subject. Radiation therapy uses high-energy radiation to shrink tumors and kill cancer cells. X-rays, gamma rays, and charged particles are types of radiation used for cancer treatment. The radiation may be delivered by a machine outside the body (external-beam radiation therapy), or it may come from radioactive material placed in the body near cancer cells (internal radiation therapy, also called brachytherapy) or by radioligand therapy. Systemic radioligand therapy uses radioactive substances, such as radioactive isotopes, that travel in the blood to kill cancer cells as described above.

### B. Diagnostic Methods

In another aspect, provided are methods of assessing eligibility of a subject for inclusion in or exclusion from a clinical trial of a B7-H3 targeted therapy using a B7-H3 antibody or antigen binding fragment thereof. The method comprises (a) measuring in a tumor sample, serum sample, cerebrospinal fluid (CSF) sample, urine sample, tear sample, or accessible fluid sample from a subject the amount of B7-H3; (b) determining if a subject has a cancer characterized as having a high level of B7-H3 expression; and (c) indicating that the subject is eligible for a clinical trial of a B7-H3 targeted therapy if the subject's cancer is characterized as having a high level of B7-H3 expression, i.e., above a predetermined threshold or that the subject is ineligible for a clinical trial of B7-H3 targeted therapy if the subject's cancer is characterized as having a low level of B7-H3 expression, i.e., below a predetermined threshold. In some instances, the threshold level is a median amount of B7-H3 determined in a reference population of patients having the same kind of cancer as the subject. In another instance, the threshold level is an optimal amount of B7-H3 determined in a reference population of patients having the same kind of cancer as the subject. “Optimal cutoff” as used herein, refers to the value of a predetermined measure on subjects exhibiting certain attributes that allow the best discrimination between two categories of an attribute. For example, finding a value for an optimal cutoff that allows one to best discriminate between two categories (subgroups) of patients for determining at least one of overall survival, time to disease progression, progression-free survival, and likelihood to respond to treatment (e.g., based on clinical assessment using the RECIST criteria, e.g., Eisenhauer, E. A., et al., Eur. J. Cancer 45:228-247 (2009) or the like as recognized in the medical field). Optimal cutoffs are used to separate the subjects with values lower than or higher than the optimal cutoff to optimize the prediction model, for example, without limitation, to maximize the specificity of the model, maximize the sensitivity of the model, maximize the difference in outcome, or minimize the p-value from hazard ratio or a difference in response.

In another aspect, provided are methods for assessing responsiveness of a subject with cancer to a B7-H3 antibody or antigen binding fragment thereof comprising: (a) measuring in a tumor sample from a subject the amount of B7-H3; (c) determining if the subject has a cancer characterized as having a high level of B7-H3 expression; and (d) indicating that the subject is more likely to respond to the B7-H3 antibody or antigen binding fragment thereof if the subject's cancer is characterized as having a high level of B7-H3 expression. Conversely, if the subject's cancer is characterized as having a low level of B7-H3 expression, the subject is less likely to respond to a B7-H3 antibody or antigen binding fragment thereof. In some instances, the amount of B7-H3 in the tumor sample is measured using a B7-H3 antibody or antigen binding fragment thereof as described herein.

In another aspect, provided are methods to diagnose cancer in a subject. Specifically, the diagnosis may be of a B7-H3 expressing cancer. The method may comprise measuring in a sample from a subject the amount of B7-H3 and diagnosing the subject with cancer if the amount of B7-H3 expression in the sample is high. In some instances, the method may comprise (a) measuring in a tumor sample from a subject the amount of B7-H3 using a B7-H3 antibody or antigen binding fragment thereof; and (c) determining if the subject has a cancer characterized as having a high level of B7-H3 expression. Conversely, if the amount of B7-H3 expression in the sample or the subject's cancer low level, the subject may not be diagnosed with cancer or may not be diagnosed with a B7-H3 expressing cancer.

In some embodiments, to diagnose cancer in a subject, or to characterize a subject's cancer, a biopsy is typically taken from a subject having an abnormal tissue growth, such as a tumor. Samples may be formalin-fixed, paraffin-embedded tissue samples obtained from the subject's cancer (tumor). In other embodiments, such as where circulating tumor cells or exosomes or target antigen (e.g., sB7-H3) are to be assessed, the sample from the subject is a blood, plasma, urine, saliva, CSF, or lymph sample. Typically, the tissue or cells of the patient sample are examined under a microscope or processed in order to confirm the diagnosis and/or assess information about the tumor. In some cases, additional tests may need to be performed on the proteins, DNA, and/or mRNA of the cells in the ample to verify the diagnosis or characterization.

In yet another aspect, provided are methods of assessing eligibility of a subject for inclusion in or exclusion from a clinical trial of a B7-H3 targeted therapy using a B7-H3 antibody or antigen binding fragment thereof. The method comprises (a) measuring in a blood sample, serum sample, cerebrospinal fluid (CSF) sample, urine sample, tear sample, or accessible fluid sample from a subject the amount of B7-H3; (b) determining if a subject has inflammation characterized as having a high level of B7-H3 expression; and (c) indicating that the subject is eligible for a clinical trial of a B7-H3 targeted therapy if the subject's inflammation is characterized as having a high level of B7-H3 expression, i.e., above a predetermined threshold or that the subject is ineligible for a clinical trial of B7-H3 targeted therapy if the subject's inflammation is characterized as having a low level of B7-H3 expression, i.e., below a predetermined threshold. In some instances, the threshold level is a median amount of B7-H3 determined in a reference population of patients having the same kind of inflammation as the subject. In another instance, the threshold level is an optimal amount of B7-H3 determined in a reference population of patients having the same kind of inflammation as the subject. “Optimal cutoff” as used herein, refers to the value of a predetermined measure on subjects exhibiting certain attributes that allow the best discrimination between two categories of an attribute. Optimal cutoffs are used to separate the subjects with values lower than or higher than the optimal cutoff to optimize the prediction model, for example, without limitation, to maximize the specificity of the model, maximize the sensitivity of the model, maximize the difference in outcome, or minimize the p-value from hazard ratio or a difference in response.

In another aspect, provided are methods for assessing responsiveness of a subject with inflammation to a B7-H3 antibody or antigen binding fragment thereof comprising: (a) measuring in a sample from a subject the amount of B7-H3; (c) determining if the subject has inflammation characterized as having a high level of B7-H3 expression; and (d) indicating that the subject is more likely to respond to the B7-H3 antibody or antigen binding fragment thereof if the subject's inflammation is characterized as having a high level of B7-H3 expression. Conversely, if the subject's inflammation is characterized as having a low level of B7-H3 expression, the subject is less likely to respond to a B7-H3 antibody or antigen binding fragment thereof. In some instances, the amount of B7-H3 in the sample is measured using a B7-H3 antibody or antigen binding fragment thereof as described herein.

In another aspect, provided are methods to diagnose inflammation in a subject. Specifically, the diagnosis may be of a B7-H3 expressing patient sample. The method may comprise measuring in a sample from a subject the amount of B7-H3 and diagnosing the subject with inflammation if the amount of B7-H3 expression in the sample is high. In some instances, the method may comprise (a) measuring in a sample from a subject the amount of B7-H3 using a B7-H3 antibody or antigen binding fragment thereof, and (c) determining if the subject has inflammation characterized as having a high level of B7-H3 expression. Conversely, if the amount of B7-H3 expression in the sample of the subject's with inflammation is a low level, the subject may not be diagnosed with inflammation or may not be diagnosed with a B7-H3 expressing inflammation.

### C. Methods of Detecting B7-H3

In another aspect, provided are methods for detecting the presence of B7-H3 expressing cells in a biological sample comprising: (a) contacting said sample with a composition comprising an isolated B7-H3 antibody or antigen binding portion thereof as described in this disclosure; and (b) detecting an amount of binding of the isolate antibody or antigen binding portion thereof as a determination of the presence of said B7-H3 expressing cells. In some embodiments, the biological sample comprises a tumor sample. In some embodiments, the composition further comprises a pharmaceutically acceptable carrier.

In some embodiments, B7-H3 expression in cancer cells or inflammation in a patient can be examined by using one or more routine biochemical analyses. In some embodiments, B7-H3 expression is determined by detecting protein expression using methods such as Western blot analysis, flow cytometry, and immunohistochemistry, ELISA, or sequencing based detection of nucleic acid labeled antibodies, or staining using a B7-H3 antibody or antigen binding portion thereof as described in this disclosure. In some instances, a combination of these methods may be used, or additional methods may also be used such as microarray analysis and RT-PCR.

In some instances, a threshold amount of B7-H3 protein expression is used to characterize B7-H3 expression as either high or low. A high level of B7-H3 protein expression refers to a measure of B7-H3 protein expression above a particular threshold. For example, the threshold may be a normal, an average, or a median amount of B7-H3 protein expression as measured in a particular set of samples, referred to as a reference population. In some instances, the reference population may be a population of normal/healthy subjects. In other instances, the reference population may be a population of subjects having a particular type of cancer (the same type of cancer that the subject being assessed has). A low level of B7-H3 expression refers to the converse of the above. For example, the threshold may be determined by identifying two distinct subgroups in the reference population by dividing samples around a mathematically determined point, such as, without limitation, a median, thus creating a subgroup whose measure is high (i.e., higher than the median) and another subgroup whose measure is low.

Also provided are methods of imaging a tumor in a subject with a B7-H3 expressing cancer, the method comprising administering to the subject an isolated antibody or antigen binding portion thereof that is specific for B7-H3 that is conjugated to an imaging label, and detecting the imaging label in the subject. Imaging methods may be used to assess tumor size and changes in tumor size over or after the course of a treatment administered to the subject. The methods may be useful to assess response of the subject to an administered treatment. In some instances, the methods may be useful to grade the subject's cancer.

Also provided are methods of monitoring response of a subject with a B7-H3 expressing cancer to cancer therapy. The methods include administering to the subject a B7-H3-specific antibody or antigen-binding fragment thereof conjugated to an imaging label at a first time point prior to the subject before the subject receives cancer therapy, detecting the imaging label in the subject to obtain a first image of the tumor, administering to the subject a B7-H3-specific antibody or antigen-binding fragment thereof conjugated to an imaging label at a second time point after the subject receives cancer therapy, detecting the imaging label in the subject to obtain a second image of the tumor; and comparing the first image to the second image to determine whether a change in tumor size has occurred. In some instances, the steps of administering to the subject a B7-H3-specific antibody or antigen-binding fragment thereof conjugated to an imaging label at a first time point after the subject receives cancer therapy, detecting the imaging label in the subject to obtain a second image of the tumor; and comparing the first image to the second image to determine whether a change in tumor size has occurred may be repeated at a third time point (or additional time points) after the subject receives cancer therapy.

Also provided are methods of imaging a subject with B7-H3 expressing inflammation, the method comprising administering to the subject an isolated antibody or antigen binding portion thereof that is specific for B7-H3 that is conjugated to an imaging label, and detecting the imaging label in the subject. Imaging methods may be used to assess inflammation size and changes in inflammation over or after the course of a treatment administered to the subject. The methods may be useful to assess response of the subject to an administered treatment.

Also provided are methods of monitoring response of a subject with a B7-H3 expressing inflammation to therapy. The methods include administering to the subject a B7-H3-specific antibody or antigen-binding fragment thereof conjugated to an imaging label at a first time point prior to the subject before the subject receives therapy, detecting the imaging label in the subject to obtain a first image of the tumor, administering to the subject a B7-H3-specific antibody or antigen-binding fragment thereof conjugated to an imaging label at a second time point after the subject receives therapy, detecting the imaging label in the subject to obtain a second image of the tumor; and comparing the first image to the second image to determine whether a change in inflammation has occurred. In some instances, the steps of administering to the subject a B7-H3-specific antibody or antigen-binding fragment thereof conjugated to an imaging label at a first time point after the subject receives therapy, detecting the imaging label in the subject to obtain a second image of the subject; and comparing the first image to the second image to determine whether a change in inflammation has occurred may be repeated at a third time point (or additional time points) after the subject receives cancer therapy. In some embodiments, the B7-H3 antibody described herein may be used to treat inflammation in a subject.

In one embodiment, a subject is administered a labeled B7-H3 antibody or antigen binding fragment thereof as described in this disclosure that is conjugated to an imaging agent. The labeled B7-H3 antibody or antigen binding fragment thereof is allowed to incubate in vivo and bind to B7-H3 in the subject's tissues. The imaging label is thereby localized to tumor cells or tissues, and the localized imaging label is detected using an appropriate imaging device as known to those skilled in the art.

The imaging agent may carry a bioluminescent or chemiluminescent label. Such labels include polypeptides known to be fluorescent, bioluminescent or chemiluminescent, or that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof. A substrate for the bioluminescent or chemiluminescent polypeptide may also be used in imaging. For example, the chemiluminescent polypeptide can be luciferase and the reagent luciferin. A substrate for a bioluminescent or chemiluminescent label can be administered before, at the same time (e.g., in the same formulation), or after administration of the agent.

In some embodiments, the imaging agent may carry a fluorescent label. For example, the fluorescent labels for use as conjugates may include, but are not limited to, Alexa 350, Alexa 430, Alexa 594, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

The imaging agent may include a paramagnetic compound, such as a polypeptide chelated to a metal (e.g., a metalloporphyrin). The paramagnetic compound may also include a monocrystalline nanoparticle, e.g., a nanoparticle including a lanthanide (e.g., Gd) or iron oxide; or a metal ion such as a lanthanide. Examples of elements that are useful in magnetic resonance imaging include gadolinium, terbium, tin, iron, or isotopes thereof.

Whole body imaging techniques using radioisotope labeled agents can be used for locating diseased cells and tissues (e.g., primary tumors and tumors which have metastasized). In some cases, the labeled agents for locating the tumor tissue or cells are administered intravenously. The bio-distribution of the label can be monitored by scintigraphy, and accumulations of the label are related to the presence of B7-H3 or other tumor markers. Whole body imaging techniques are described in, e.g., U.S. Pat. Nos. 4,036,945 and 4,311,688.

An image according to this disclosure can be generated by computer assisted tomography (CAT), magnetic resonance spectroscopy (MRS) image, magnetic resonance imaging (MRI), positron emission tomography (PET), single-photon emission computed tomography (SPECT), fluorescent, photoacoustic, or bioluminescence imaging (BLI) or equivalent.

Computer assisted tomography (CAT) and computerized axial tomography (CAT) systems and devices well known in the art can be used to generate an image. (See, for example, U.S. Pat. Nos. 6,151,377; 5,946,371; 5,446,799; 5,406,479; 5,208,581; and 5,109, 97.) The imaging methods may also utilize animal imaging modalities, such as MicroCAT™. (ImTek, Inc.).

Magnetic resonance imaging (MRI) systems and devices well known in the art can be used for imaging. For a description of MRI methods and devices, see, for example, U.S. Pat. Nos. 6,151,377. MRI and supporting devices are commercially available, for example, from Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics.

Positron emission tomography imaging (PET) systems and devices well known in the art can be used for imaging. For example, an imaging method of this disclosure may use the system designated PET VI located at Brookhaven National Laboratory. For descriptions of PET systems and devices, see, for example, U.S. Pat. Nos. 6,151,377. Animal imaging modalities such as micro-PETs (Concorde Microsystems, Inc.) can also be used.

Single-photon emission computed tomography (SPECT) systems and devices well known in the art can be used for imaging. (See, for example, U.S. Pat. Nos. 6,115,446; 6,072,177; 5,608,221; 5,600,145; 5,210,421; 5,103,098.) Imaging methods may also use animal imaging modalities, such as micro-SPECTs.

Sensitive photon detection systems can be used to detect bioluminescent and fluorescent proteins externally; see for example, Contag (2000), Neoplasia 2:41-52; and Zhang (1994), Clin. Exp. Metastasis, 12:87-92. The imaging methods of the disclosure can be practiced using any such photon detection device, for example, an intensified charge-coupled device (ICCD) camera coupled to an image processor. Photo detection devices are also commercially available from Xenogen/Perkin-Elmer, Hamamatsue.

Disclosed herein are materials, compositions, and methods that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are various additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### EXAMPLES

The following examples are offered to illustrate, but not to limit, the claimed methods and compositions.

### Example 1. Analysis of MIL33B Chimeric Antibody

A humanized MIL33B antibody was generated based on the murine MIL33B antibody sequence (i.e., a murine antibody that specifically binds B7-H3 as described in PCT Publication No. WO 2021/101991). The DNA sequences encoding the chimeric antibody heavy and light chains, as listed in Table 6, were synthesized, and inserted into pcDNA3.4 vector to construct expression plasmids of full-length IgGs. Expression of chimeric antibody was conducted in Expi293 cell culture, and the supernatants were purified with protein A affinity column. The purified antibody was buffer-exchanged into PBS using PD-10 desalting column. The concentration and purity of the purified proteins were determined by OD280 and SDS-PAGE, respectively. Binding confirmation and affinity were evaluated by Surface Plasmon resonance (SPR) using Biacore™ 8K/T200.

TABLE 6
VH and VL nucleotide sequences for chimeric and grafted antibody.

| Antibody | | |
|---|---|---|
| ID | VH sequence | VL sequence |
| Chimeric | SEQ ID NO: 5 | SEQ ID NO: 6 |
| | ATGGGCTGGTCATGCATTATTCTGTTTCTGGT | ATGGGCTGGTCATGTATTATTCTGTTT |
| | CGCAACTGCTACAGGCGTGCATAGTGAGGTG | CTGGTCGCAACTGCTACAGGGGTCCA |
| | CAGCTGCAGCAGAGCGGACCTGAGCTGGTCA | TAGTCAGATCGTGCTGACCCAGAGCC |
| | AGCCCGGCGCTTCTGTGAAAATGAGCTGTAA | CTGCCATCATGAGCGCCTCCCCTGGC |
| | AGCCAGCGGCTACACCTTCACCTCTTATGTG | GAGAAGGTGACCATGACCTGTAGCG |
| | ATGCACTGGGTGCGGCAGTCTCCTGGACAAG | TGTCCAGCTCTGTGAACTACATGCAC |
| | GCCTGGAAGGCATCGGCTACATCAACAGCTA | TGGTATCAGCAGAAGAGCGGCACAT |
| | CAGCGACGGCACAAAGTACAACGAGAAGTT | CTCCAAAGCGGTGGATCTACGATACC |
| | CAAGGGCAAGGCTACACTGACCTCCGATAAG | TCTAAACTGGCCAGCGGCGTCCCCGC |
| | TCCAGCTCTACAGCCTACATGGAACTGAGCG | TAGATTCAGTGCCTCTGGAAGCGGAA |
| | GCCTGACCAGCGAGGACAGCGCCGTGTACTA | CCAGCTACAGCCTGACAATCAGCAGC |
| | CTGCGCCAGATGGGGAGGACTGGGCAATGGC | ATGGAAGCCGAGGACGCCGCTACAT |
| | GCCATGGACTATTGGGGCCAGGGCACCAGCG | ACTACTGCCAGCAATGGACAAGCAA |
| | TGACCGTGTCCAGCGCCAGCACCAAGGGCCC | CCCTCTGACCTTCGGCGCCGGCACCA |
| | TTCCGTGTTTCCACTGGCCCCCTCCTCTAAAT | AGCTGGAACTGAAGAGGACAGTGGC |
| | CCACATCTGGCGGCACCGCCGCCCTGGGCTG | CGCCCCAAGCGTGTTCATCTTTCCCC |
| | TCTGGTGAAGGACTACTTCCCAGAGCCTGTG | CTTCCGACGAGCAGCTGAAGTCTGGC |
| | ACAGTGTCCTGGAACTCTGGCGCCCTGACAT | ACCGCCAGCGTGGTGTGCCTGCTGAA |
| | CCGGCGTGCACACATTTCCAGCCGTGCTGCA | CAACTTCTACCCTCGGGAGGCCAAGG |
| | GAGCTCCGGCCTGTACAGCCTGTCTAGCGTG | TCCAGTGGAAGGTGGATAACGCCCTG |
| | GTGACAGTGCCCTCCTCTAGCCTGGGCACAC | CAGTCTGGCAATAGCCAGGAGTCCGT |
| | AGACCTATATCTGCAACGTGAATCACAAGCC | GACCGAGCAGGACTCTAAGGATAGC |
| | AAGCAATACCAAGGTGGACAAGAAGGTGGA | ACATATTCCCTGTCTAGCACCCTGAC |
| | GCCCAAGTCCTGTGATAAGACACACACCTGC | ACTGAGCAAGGCCGATTACGAGAAG |
| | CCCCCTTGTCCTGCTCCCGAGCTGCTGGGCGG | CACAAGGTGTATGCCTGTGAAGTCAC |
| | CCCTAGCGTGTTCCTGTTTCCACCCAAGCCTA | CCATCAGGGGCTGTCATCACCCGTCA |
| | AGGACACCCTGATGATCTCCCGGACACCCGA | CTAAGTCATTCAATCGCGGAGAATGC |
| | GGTGACCTGCGTGGTGGTGGACGTGTCTCAC | TGATAA |
| | GAGGATCCTGAGGTGAAGTTCAACTGGTATG | |
| | TGGATGGCGTGGAGGTGCACAATGCCAAGAC | |
| | CAAGCCCAGAGAGGAGCAGTACAACTCTACA | |
| | TATAGGGTGGTGAGCGTGCTGACCGTGCTGC | |
| | ACCAGGACTGGCTGAACGGCAAGGAGTATA | |
| | AGTGCAAGGTGTCCAATAAGGCCCTGCCCGC | |
| | CCCCATCGAGAAGACAATCAGCAAGGCCAA | |
| | GGGCCAGCCTCGGGAGCCACAGGTGTACACC | |
| | CTGCCTCCATCCAGAGACGAGCTGACAAAGA | |
| | ACCAGGTGTCTCTGACATGTCTGGTGAAGGG | |
| | CTTCTATCCTAGCGATATCGCCGTGGAGTGG | |
| | GAGTCCAATGGCCAGCCAGAGAACAATTACA | |
| | AGACCACACCCCCTGTGCTGGACTCCGATGG | |
| | CTCCTTCTTTCTGTATTCCAAGCTGACCGTGG | |
| | ATAAGTCTCGGTGGCAGCAGGGCAACGTGTT | |
| | CAGCTGTTCCGTGATGCACGAAGCCCTGCAT | |
| | AATCACTATACTCAGAAATCCCTGTCCCTGTC | |
| | ACCTGGAAAGTGATAA | |
| Grafted | SEQ ID NO: 7 | SEQ ID NO: 8 |
| | ATGGGCTGGTCATGCATTATTCTGTTTCTGGT | ATGGGCTGGTCATGTATTATTCTGTTT |
| | CGCAACTGCTACAGGCGTGCATAGTCAGGTG | CTGGTCGCAACTGCTACAGGGGTCCA |
| | CAGCTGGTGCAGAGCGGCGCTGAGGTGAAG | TAGTGAGATCGTGCTGACCCAGAGCC |
| | AAGCCCGGCGCCAGCGTGAAAGTGTCTTGTA | CAGCCACACTGAGCCTGAGCCCTGGC |
| | AAGCCTCTGGCTACACCTTCACCAGCTACGT | GAGCGGGCCACCCTCTCCTGCAGCGT |
| | GATGCACTGGGTCAGACAGGCCCCTGGACA | GTCCAGCTCCGTCAACTACATGCACT |
| | GCGGCTGGAATGGATGGGCTATATCAACAGC | GGTATCAGCAGAAACCTGGCCAGGC |
| | TACAGCGACGGCACCAAGTACAACGAGAAG | CCCTAGACTGCTGATCTACGACACCA |
| | TTCAAGGGCAGAGTGACAATCACCCGGGAC | GCAAGCTGGCCTCTGGAATCCCTGCT |
| | ACCAGCGCCAGCACAGCCTACATGGAACTGT | AGATTCAGCGGATCTGGCAGCGGCA |
| | CTAGCCTGAGAAGCGAGGATACCGCCGTGTA | CCGATTTTACCCTGACAATCTCTAGC |
| | CTACTGCGCCAGATGGGGCGGCCTGGGCAAT | CTGGAACCCGAGGACTTCGCCGTGTA |
| | GGCGCTATGGACTATTGGGGACAAGGAACC | CTACTGTCAGCAATGGACCAGCAACC |
| | CTGGTTACAGTGTCCTCCGCCAGCACCAAGG | CCCTGACATTCGGCCAGGGCACCAAG |
| | GCCCTTCCGTGTTTCCACTGGCCCCCTCCTCT | CTGGAAATCAAGAGGACAGTGGCCG |
| | AAATCCACATCTGGCGGCACCGCCGCCCTGG | CCCCAAGCGTGTTCATCTTTCCCCCTT |
| | GCTGTCTGGTGAAGGACTACTTCCCAGAGCC | CCGACGAGCAGCTGAAGTCTGGCAC |
| | TGTGACAGTGTCCTGGAACTCTGGCGCCCTG | CGCCAGCGTGGTGTGCCTGCTGAACA |
| | ACATCCGGCGTGCACACATTTCCAGCCGTGC | ACTTCTACCCTCGGGAGGCCAAGGTC |
| | TGCAGAGCTCCGGCCTGTACAGCCTGTCTAG | CAGTGGAAGGTGGATAACGCCCTGC |
| | CGTGGTGACAGTGCCCTCCTCTAGCCTGGGC | AGTCTGGCAATAGCCAGGAGTCCGTG |
| | ACACAGACCTATATCTGCAACGTGAATCACA | ACCGAGCAGGACTCTAAGGATAGCA |
| | AGCCAAGCAATACCAAGGTGGACAAGAAGG | CATATTCCCTGTCTAGCACCCTGACA |
| | TGGAGCCCAAGTCCTGTGATAAGACACACAC | CTGAGCAAGGCCGATTACGAGAAGC |
| | CTGCCCCCCTTGTCCTGCTCCCGAGCTGCTGG | ACAAGGTGTATGCCTGTGAAGTCACC |
| | GCGGCCCTAGCGTGTTCCTGTTTCCACCCAA | CATCAGGGGCTGTCATCACCCGTCAC |
| | GCCTAAGGACACCCTGATGATCTCCCGGACA | TAAGTCATTCAATCGCGGAGAATGCT |
| | CCCGAGGTGACCTGCGTGGTGGTGGACGTGT | GATAA |
| | CTCACGAGGATCCTGAGGTGAAGTTCAACTG | |
| | GTATGTGGATGGCGTGGAGGTGCACAATGCC | |
| | AAGACCAAGCCCAGAGAGGAGCAGTACAAC | |
| | TCTACATATAGGGTGGTGAGCGTGCTGACCG | |
| | TGCTGCACCAGGACTGGCTGAACGGCAAGG | |
| | AGTATAAGTGCAAGGTGTCCAATAAGGCCCT | |
| | GCCCGCCCCCATCGAGAAGACAATCAGCAA | |
| | GGCCAAGGGCCAGCCTCGGGAGCCACAGGT | |
| | GTACACCCTGCCTCCATCCAGAGACGAGCTG | |
| | ACAAAGAACCAGGTGTCTCTGACATGTCTGG | |
| | TGAAGGGCTTCTATCCTAGCGATATCGCCGT | |
| | GGAGTGGGAGTCCAATGGCCAGCCAGAGAA | |
| | CAATTACAAGACCACACCCCCTGTGCTGGAC | |
| | TCCGATGGCTCCTTCTTTCTGTATTCCAAGCT | |
| | GACCGTGGATAAGTCTCGGTGGCAGCAGGG | |
| | CAACGTGTTCAGCTGTTCCGTGATGCACGAA | |
| | GCCCTGCATAATCACTATACTCAGAAATCCC | |
| | TGTCCCTGTCACCTGGAAAGTGATAA | |

The affinity of the chimeric antibody to the B7-H3 antigen was determined using Surface Plasmon Resonance biosensor, Biacore 8K/T200 (GE Healthcare). Antibody was captured to the sensor chip through Fc capture method. B7-H3 antigen was used as the analyte. The data of dissociation (kd) and association (ka) rate constants were obtained using Biacore 8K/T200 evaluation software. The equilibrium dissociation constants (KD) were calculated from the ratio of kd over ka. Binding kinetics were evaluated using SPR according to the following parameters. The system had a capture time of 30 (s), at a flow rate of 10 (μl/min). Association contact time was 120 seconds with a dissociation contact time of 360 seconds at a flow rate of 30 (μl/min) and sample concentrations of 1.171875, 2.34375, 4.6875, 9.375, 18.75, 37.5, and 75 nM. The resulting binding kinetics of the chimeric antibody were as follows: a ka of 6.09E05 (1/Ms), a kd of 1.21E-04 (1/s), a KD of 1.99E-10 (M), and an Rmax of 65.4 (RU).

Following testing and validation of the chimeric antibody as described in this Example, the grafted antibody also was tested according to similar methods (data not shown).

### Example 2. Generating Humanized B7-H3 Antibody Libraries

A library of point mutations was generated that included the framework region around the CDR that targeted the least conserved mutations from the mouse framework compared to the human framework. The humanized Fast Screening for Expression Biophysical-properties and Affinity (FASEBA) screening library that contained all back mutants in the inner core of the antibody structure (Fab) was designed under contract by GenScript. Construction of the library was carried out following GenScript's standard operating procedures (SOP).

Specifically, a total of 48 clones for both the VH and VL regions were generated for sequencing. Analysis of the sequences revealed 41 VL sequences for further testing with 7 bad sequences and 38 VH sequences for further testing with 10 bad sequences. Through sequence alignments of the clones, a total of 8 unique VL sequences and 14 unique VH sequences were identified. FASEBA screening against antigen protein was conducted. The individual Fab clones were expressed in a 96-well plate and crude protein secreted by E. coli into the medium was assayed by ELISA against bovine serum albumin (BSA) and the target antigen protein for the assessment of expression and binding activity. The 15 clones that demonstrated the best binding affinity were selected for DNA sequencing (i.e., AHF15937, AHF15938, AHF15939, AHF15940, AHF15941, AHF15942, AHF15943, AHF15944, AHF15945, AHF15946, AHF15947, AHF15948, AHF15949, AHF15950, and AHF15951). Of the 15 listed, 11 unique clones were used for binding affinity ranking.

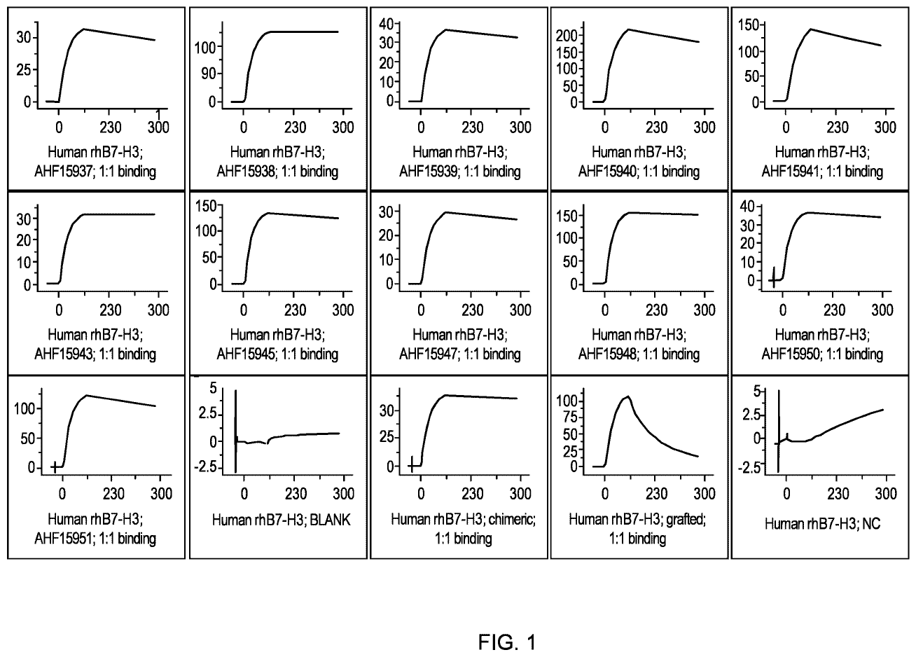

For affinity ranking, albumin was immobilized onto the sensor chip using the amine coupling method. The selected Fab-single-domain fused to a single-domain antibody against serum albumin (Fab-SASA) secreted into the culture medium was injected and captured by albumin on the chip (capture phase). After equilibration, B7-H3 antigen was injected for 180 seconds (association phase) followed by the injection of running buffer for 420 seconds (dissociation phase). The surface was regenerated before the injection of another Fab-SASA clone. The process was repeated until all Fab-SASA clones were analyzed. Responses of reference flow cells were subtracted from the Fab-SASA flow cells during each cycle. The off-rates of Fab-SASA clones were obtained from fitting the experimental data locally to a 1:1 interaction model using the Biacore 8K/T200 evaluation software. The Fab-SASA clones were ranked by their dissociation rate constants (off-rates, kd). The clones that have similar or higher binding affinity than the chimeric Fab-SASA were sequenced. Based on the sequencing results, the clones with higher affinity and less back mutation sites were selected. The results of the binding affinity for the 11 clones selected can be seen in Table 7 below with the corresponding sensor-grams shown in FIG. 1.

TABLE 7
Binding affinity for 11 selected Fab-SASA clones.

| | | | | | | Rmax |
|---|---|---|---|---|---|---|
| Ligand | Analyte | Chi²⁻ (RU²) | ka (1/Ms) | kd (1/s) | KD (M) | (RU) |
| AHF15937 | Human rhB7-H3 | 3.91E−01 | 2.45E05 | 4.30E−04 | 1.76E−09 | 60.9 |
| AHF15938 | Human rhB7-H3 | 5.87E00 | 2.45E05 | 1.97E−06 | 8.04E−12 | 135.1 |
| AHF15939 | Human rhB7-H3 | 1.78E−01 | 2.63E05 | 3.04E−04 | 1.16E−09 | 38.4 |
| AHF15940 | Human rhB7-H3 | 7.30E00 | 2.24E05 | 5.06E−04 | 2.26E−09 | 237.9 |
| AHF15941 | Human rhB7-H3 | 4.74E−01 | 1.62E05 | 7.07E−04 | 4.35E−09 | 167.3 |
| AHF15943 | Human rhB7-H3 | 2.75E−01 | 2.41E05 | 1.00E−06 | 4.15E−12 | 33.6 |
| AHF15945 | Human rhB7-H3 | 2.13E00 | 2.74E05 | 2.44E−04 | 8.88E−10 | 140.6 |
| AHF15947 | Human rhB7-H3 | 1.07E−01 | 2.33E05 | 2.88E−04 | 1.23E−09 | 31.8 |
| AHF15948 | Human rhB7-H3 | 2.68E00 | 2.55E05 | 9.35E−05 | 3.66E−10 | 164.1 |
| AHF15950 | Human rhB7-H3 | 1.28E−01 | 2.71E05 | 1.96E−04 | 7.20E−10 | 38.2 |
| AHF15951 | Human rhB7-H3 | 1.31E00 | 2.56E05 | 4.11E−04 | 1.60E−09 | 128.4 |
| Blank | Human rhB7-H3 | NA | NA | NA | NA | NA |
| Chimeric | Human rhB7-H3 | 5.36E−01 | 2.62E05 | 1.31E−04 | 4.99E−10 | 65.9 |
| Grafted | Human rhB7-H3 | 1.21E00 | 4.13E05 | 1.17E−02 | 2.85E−08 | 140 |
| NC (BSA) | Human rhB7-H3 | NA | NA | NA | NA | NA |

### Example 3. Constructing and Producing Humanized IgGs

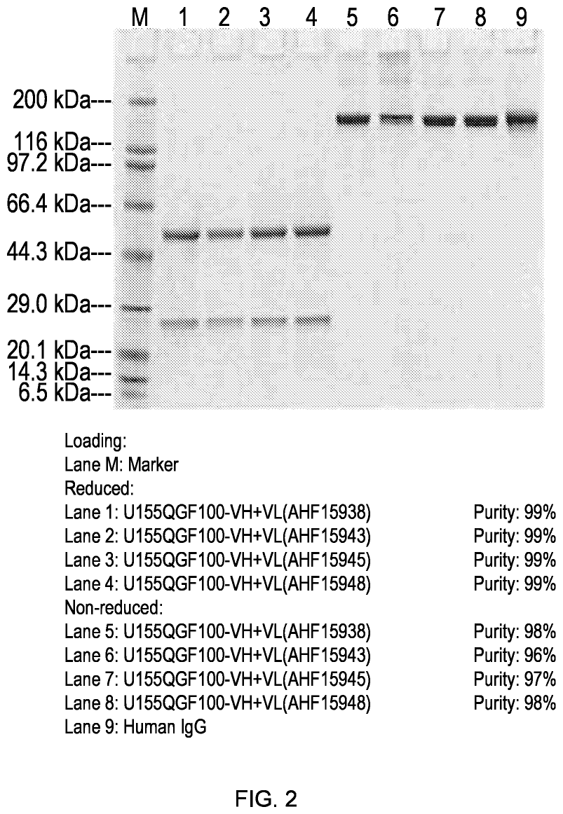

From the affinity ranking in Table 7, five clones were selected for full-length IgG1 expression, purification, and further study. The DNA sequences encoding the top binder sequences were inserted into pcDNA3.4 vector to make expression plasmids of full-length IgGs. The heavy and light chain expression plasmids were used to co-transfect Expi293 cells. The recombinant IgGs secreted into the medium were purified using protein A affinity chromatography following GenScript's SOP. Western blot analysis was performed on the five humanized antibodies, as shown in FIG. 2. The purified IgGs migrated as about 150 kDa bands in sodium dodecyl-sulfate polyacrylamide gel electrophoresis (SDS-PAGE) under non-reducing conditions and around 50 kDa and 25 kDa bands under reducing conditions.

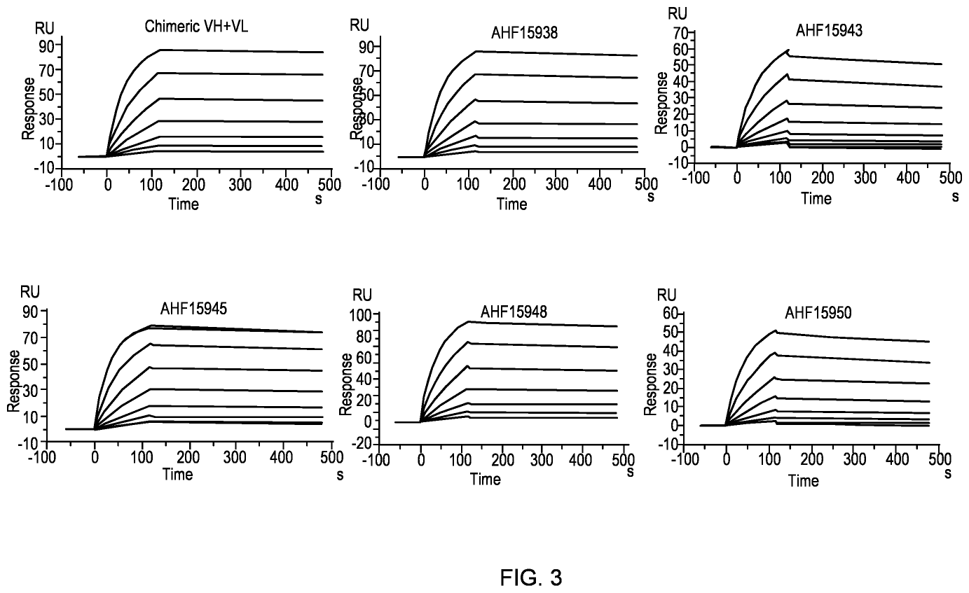

The 5 humanized antibodies were purified and tested for binding affinity to human rhB7-H3. Binding confirmations were tested by Surface Plasmon Resonance (SPR) using Biacore 8K/T200. The binding kinetics of the humanized antibodies can be found in Table 8, below, and corresponding sensor-grams found in FIG. 3. Three humanized monoclonal antibodies were further tested based on the binding affinity results in Table 8 (i.e., AHF15945 (also known as MIL33B-H1), AHF15938 (also known as MIL33B-H2), and AHF15948 (also known as MIL33B-H3)).

TABLE 8
Binding affinity for humanized antibodies to rhB7-H3 by SPR.

| | | | | | | Rmax |
|---|---|---|---|---|---|---|
| Ligand | Analyte | Chi²⁻ (RU²) | ka (1/Ms) | kd (1/s) | KD (M) | (RU) |
| Chimeric | Human rhB7-H3 | 0.0523 | 4.82E05 | 7.16E−05 | 1.48E−10 | 92.06 |
| VH + VL | | | | | | |
| AHF 15938 | Human rhB7-H3 | 0.0557 | 4.64E05 | 1.05E−04 | 2.26E−10 | 91.59 |
| AHF 15945 | Human rhB7-H3 | 0.0801 | 5.97E05 | 1.39E−04 | 2.33E−10 | 79.99 |
| AHF 15948 | Human rhB7-H3 | 0.0652 | 4.81E05 | 1.17E−04 | 2.43E−10 | 100.7 |
| AHF 15950 | Human rhB7-H3 | 0.0583 | 4.15E05 | 2.83E−04 | 6.79E−10 | 55.74 |
| AHF 15943 | Human rhB7-H3 | 0.0793 | 3.84E05 | 2.92E−04 | 7.60E−10 | 62.54 |

### Example 4. Humanized MIL33B Antibodies Selectively Bind to Human 4Ig-B7-H3 vs 2Ig-B7-H3

The in vitro binding affinity of three humanized MIL33B antibodies for human 4Ig-B7-H3 and 2Ig-B7-H3 were assessed by biolayer interferometry. For comparison, MIL33B-mIgG2a (murine antibody) and the chimeric antibody were run in tandem. The extracellular domains of the indicated 4Ig-B7-H3 and 2Ig-B7-H3 proteins were purchased from R&D systems. All proteins were validated by the vendor to be both pure and functional. KD values were determined utilizing capture biolayer interferometry (Octet, Molecular Devices) in “affinity” mode, wherein the MILB33 antibody was captured on the tip of the probe and placed into solutions containing different concentrations of target B7-H3 extracellular domain. KD, KD error, and R² values are reported in Table 9. Data demonstrated that the derived human MIL33B antibodies (MIL33B-H1, MIL33B-H2, or MIL33B-H3) maintained high affinity to B7-H3 with higher selectivity for 4Ig-B7-H3 vs 2Ig-B7-H3 when compared to the murine MIL33B antibody. MIL33B-H3 hIgG1 demonstrated the highest selectivity, with a 4Ig-B7-H3 over 2Ig-B7-H3 ratio of 360, a property that may prove critical for successful use of the antibody in vivo.

TABLE 9
Binding of various MIL33B antibodies to the extracellular
domain of human 4Ig vs 2Ig B7-H3.

| | | Antibodies | | | | |
|---|---|---|---|---|---|---|
| | | MIL33B- | MIL33B- | MIL33B-H1- | MIL33B-H2- | MIL33B-H3- |
| | | mIgG2a | chimera | hIgG1 | hIgG1 | hIgG1 |
| 4Ig-B7- | KD (M) | 8.07E−11 | 1.60E−10 | 1.53E−10 | 1.20E−10 | 1.21E−10 |
| H3 | KD error | 9.59E−12 | 1.45E−11 | 1.20E−11 | 1.17E−11 | 2.16E−11 |
| protein | (M) | | | | | |
| | R² | 0.961 | 0.954 | 0.977 | 0.980 | 0.973 |
| 2Ig-B7- | KD (M) | 7.81E−10 | 1.32E−08 | 2.16E−08 | 1.40E−08 | 4.35E−08 |
| H3 | KD error | 4.39E−11 | 5.90E−11 | 5.24E−10 | 7.04E−11 | 3.39E−10 |
| protein | (M) | | | | | |
| | R² | 0.9523 | 0.9985 | 0.9771 | 0.9964 | 0.995 |

### Example 5. Humanized MIL33B-H3 Binds to 4Ig Porcine and Cynomolgus Protein

The extracellular domains of the indicated 4Ig-B7-H3 and 2Ig-B7-H3 proteins were purchased from R&D systems. All proteins were validated by the vendor to be both pure and functional. KD values were determined utilizing capture biolayer interferometry (Octet, Molecular Devices) in “affinity” mode, wherein the MILB33 antibody was captured on the tip of the probe and placed into solutions containing different concentrations of target extracellular domain. KD, KD error, and R² values are reported in Table 10. The data demonstrated that the preferred human MIL33B antibody (MIL33B-H3) had comparable binding to porcine and cynomolgus 4Ig-B7-H3 proteins as the previously reported mouse antibody (MIL33B-mIgG2a). Additionally, these results demonstrated that the humanized MIL33B antibody binds with sub nanomolar affinity to Cynomolgus and Porcine 4Ig-B7-H3.

TABLE 10
Binding of MIL33B-H3-hIgG1 and MIL33B-mIgG2a antibodies
to Porcine and Cynomolgus 4Ig-B7-H3 protein.

| | | Antibodies | |
|---|---|---|---|
| | | MIL33B-mIgG2a | MIL33B-H3-hIgG1 |
| Porcine | KD (M) | 1.02E−10 | 5.59E−10 |
| 4Ig-B7H-3 | KD error (M) | 1.46E−11 | 1.20E−11 |
| protein | R² | 0.9893 | 0.9931 |
| Cynomolgus | KD (M) | — | 2.80E−10 |
| 4Ig-B7H-3 | KD error (M) | — | 1.03E−11 |
| protein | R² | — | 0.998 |

### Example 6. Fluorescence Microscopy of Tumor Cell Binding

Net tumor cell binding and retention were studied using a human HeLa tumor cell binding model and MIL33B-H3 conjugated with the fluorescent tag AlexaFluor594. Fluorescence microscopy of live cells confirmed that the MIL33B-H3 antibody binds human tumor cells more intensely compared to the murine MIL33B antibody (data not shown).

### Example 7. Generating and Characterizing MIL33B-Specific CAR T Cells

To generate CAR cells, healthy donor cells can be lentivirally transduced with an anti-CD276 (4Ig-B7-H3)-specific CAR containing the MIL33B-H3 variable region sequences (e.g., SEQ ID NO: 42), CD8a hinge, CD8a transmembrane, and 4-1BB and CD3ζ signaling domains. To evaluate surface expression of anti-CD276 CAR, flow cytometry can be employed using FITC-conjugated CD276 extracellular domain protein to evaluate the transduction efficiency. These are mostly commonly CAR-T cells, but could be leveraged with CAR-NK cells, CAR-macrophages, and CAR-neutrophils.

Anti-CD276 CAR cells (e.g., CAR-T cells) can be co-cultured with CD276+ B-cell lymphoma cell lines (e.g., Jeko-1, sp53, and CA46) and CD276-negative NK cell leukemia cell lines (e.g., NK92). Under co-culture conditions, it is understood that the anti-CD276 CAR-T cells may induce cell death (lysis) in CD276+ cells while not in parental cells or CD276-negative cell lines.

### Example 8. M1 and M2 Macrophages Express Human 4Ig-B7-H3

To generate M1 and M2 macrophages, 150,000 THP-1 cells were seeded in 200 μL of RPMI-1640 medium containing 10% FBS, 1% L-Glu, and 0.05 mM 2-mercaptoethanol with or without polarization cytokines (M1-like: IFNγ 20 ng/mL; 250 ng/mL LPS) for 48 hours. After incubating at 37° C. 5% CO₂, cells were activated using a final concentration of 2.5 μg/μL PMA or 4 mM. For imaging, cells were fixed using 4% PFA for 10 minutes and washed two times with PBS. Immunofluorescence staining was performed using AF594-labeled MIL33B for 1 hour at 4° C. DAPI staining of nuclei was performed for 10 minutes at room temperature. Cells were washed three times with PBS and epifluorescence images were captured using a fluorescence microscope (Nikon TiE) equipped with a Hamamatsu Orca flash 4 camera.

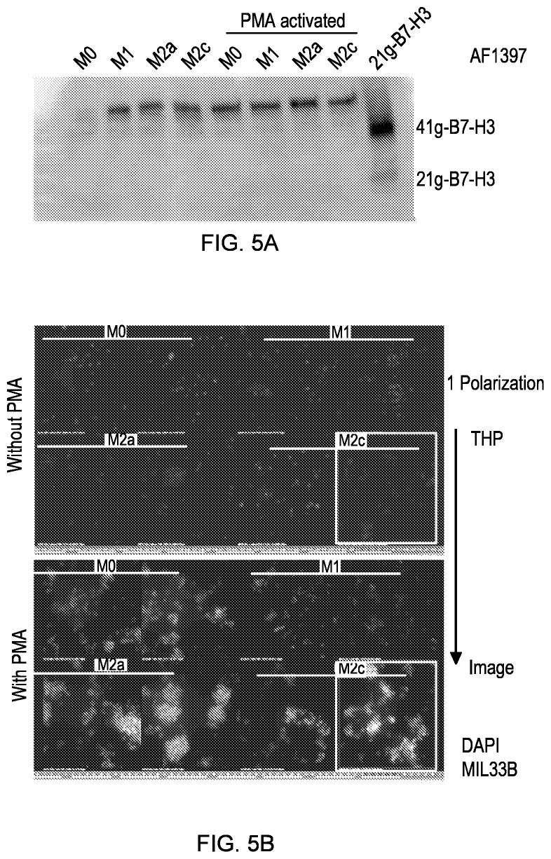

Western blot analysis was performed according to standard protocols to assess the molecular weight of 4Ig-B7-H3 and 2Ig-B7-H3. FIG. 5A shows the Western blot analysis of 4Ig-B7-H3 and 2Ig-B7-H3 expression in non-activated leukemia monocytic cells (THP-1) in M0, M1, M2a, and M2c-like states and phorbol myristate acetate (PMA) activated THP-1 cells in M0, M1, M2a, and M2c-like states. In brief, the M1 and M2 macrophages both express human 4Ig-B7-H3. While not being bound by any theory, it is believed that the larger than expected molecular weight is due to changes in the core glycosylation with a weaker secondary band consistent with 4Ig-B7-H3 as found on most tumors.

FIG. 5B shows a panel of microscopy images of immunofluorescence staining of THP-1 cells in M0, M1, M2a, and M2c-like states and treated with (bottom panels) or without (top panels) PMA and further incubated with MIL33B antibody. In brief, MIL33B still binds to the epitope in live cells in all states with higher expression in PMA-activated states. Additionally, the results demonstrate the possibility for using the antibodies described herein having antibody-dependent cellular cytotoxicity (ADCC), antibody-dependent cellular phagocytosis (ACDP), or acting as a blocking antibody to target activated myeloid cells in hemophagocytic lymphohistiocytosis (HLH).

### Example 9. Humanized MIL33B-H3 in Treatment of Reactive Oxygen and Nitrogen Species (RONS) Bursts

To assess the potential for M1 and M2 macrophage generation in THP-1 cells, 150,000 THP-1 cells were seeded in 200 μL of RPMI-1640 medium containing 10% FBS, 1% L-Glu, and 0.05 mM 2-mercaptoethanol with or without polarization cytokines (M1-like: IFNγ 20 ng/mL; 250 ng/mL LPS) for 48 hours. After incubating at 37° C. 5% C02, the cells were activated using a final concentration of 2.5 μg/μL PMA and imaged using 100 M L-012 in Modified Earl's Balanced Salt Solution (MEBSS). Bioluminescence images were captured with a supercooled CCD camera (IVIS Spectrum), images were acquired every 5 minutes over a 30-minute timeframe with 300 s exposure.

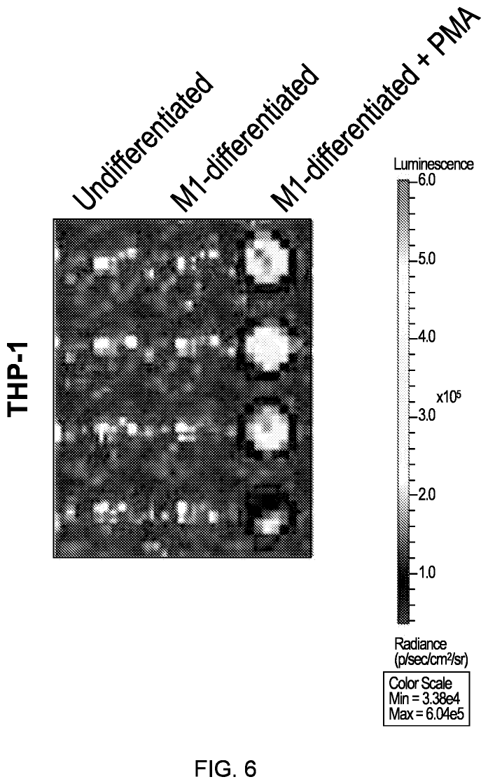

THP-1 cells differentiated into an M1 like state with LPS and interferon gamma treatment did not have respiratory burst similar to the undifferentiated state, while the THP-1 cells differentiated into an M1 like state and further treated with PMA was higher when compared to both the non-treated PMA macrophages and the undifferentiated population (FIG. 6).

### Example 10. Humanized MIL33B-H3 in Treatment of RONS Bursts in Murine Models

### RONS Burst Imaging Protocol

Mice were pre-imaged using intraperitoneal injection of L-012 sodium salt in saline (20 mg kg-1). Whole animal imaging was performed in bioluminescence and reflectance mode 10 minutes after injection of L-012 on a bioluminescence imaging system (IVIS Spectrum). Images were acquired with a 19-cm FOV, F/1.1, binning 4×4, 300 s or 5 minutes.

### Imaging and Treatment Protocol

17-week old SGM3 mice obtained from Jackson Labs (701362) were pre-imaged for RONS prior to treatment initiation. Upon treatment initiation, mice were treated twice weekly with 200 μL PBS, or 200 μL H3-MIL33B (Final dose 200 g/mouse/treatment, intraperitoneal injections). Mice were imaged again on days 7 and 14. After this point treatment was stopped. During the two-week treatment pause, mice were imaged on days 21 and 28. Treatment was re-initiated for 1 week, followed by a final whole animal L-012 imaging on day 35.

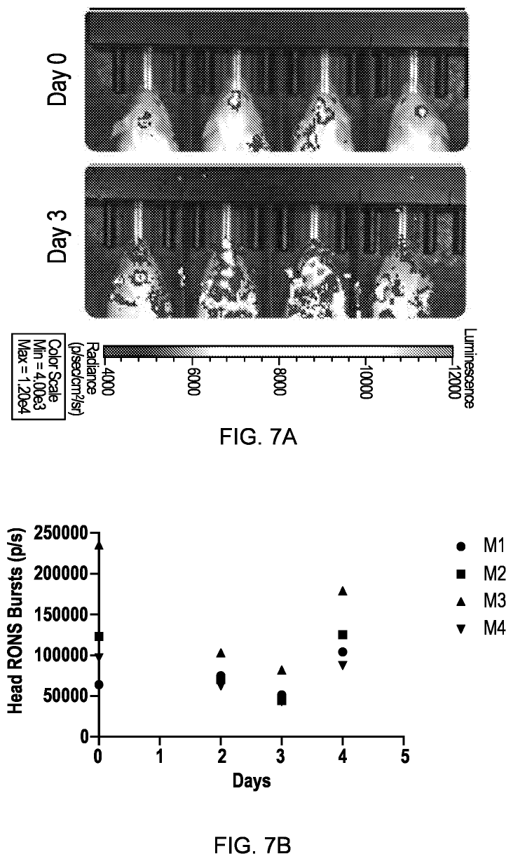

FIG. 7A provides a panel of SGM3 mice intraperitoneally injected with L-012 sodium salt in saline and pre-imaged (Day 0) and subsequently imaged following 3 days of incubation (day 3). The SGM3 mice treated with PBS only showed expression of L-012 above camera background. FIG. 7B is a graph of the head reactive oxygen and nitrogen species (RONS) bursts following 5 days of treatment in SGM3 mice injected intraperitoneally with L-012 sodium salt in saline as determined from the mouse model. Taken together, the results revealed that HLH occurs in the brain spontaneously over time in the mouse model. These findings are consistent with the literature and set a baseline for the studies performed below.

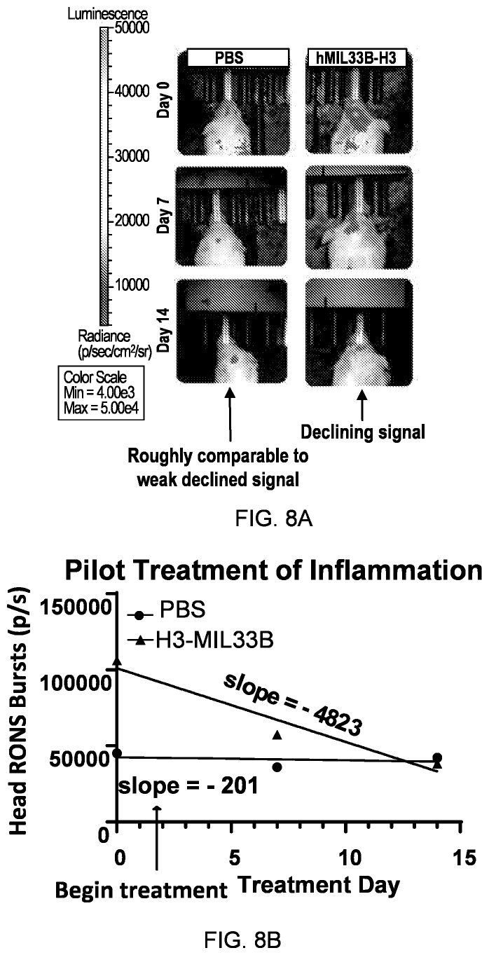

Following three days of incubation, the mice were treated with hMIL33B-H3 antibody on day 7 and day 14. FIG. 8A provides a panel of SGM3 mice injected intraperitoneally with 200 μg of humanized hMIL33B-H3 in phosphate buffer saline (PBS) two times per week for 14 days or SGM3 mice injected intraperitoneally with PBS buffer as a control group. FIG. 8B provides a graph of the head RONS bursts in SGM3 mice at Day 0, Day 7, and Day 14. The slope of the line for mice treated with hMIL33B-H3 is −4823 (photons/(sec*day)) while in the control group the slope is −201 (photons/(sec*day)). The signal in L-012 in mice treated with the hMIL33B-H3 antibody decreased indicating the reduced RONS in mice treated with the hMIL33B-H3. The mice treated with PBS as a control group did not have a declined signal intensity in RONS.

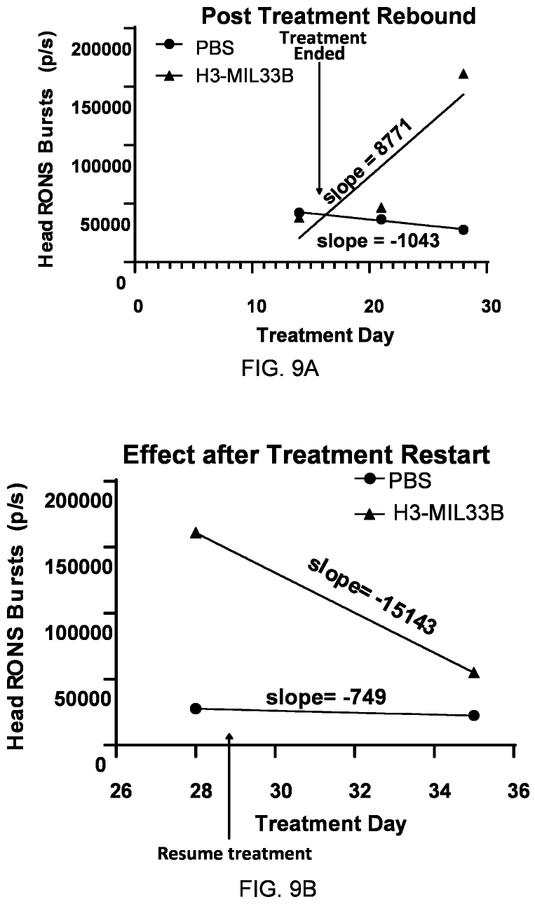

After day 14 of treatment, treatment was halted and RONS bursts were measured on day 21 and day 28 to evaluate if RONS generated in the brain re-escalates. FIG. 9A provides a graph of the head RONS burst in SGM3 mice post treatment with the hMIL33B-H3 antibody or PBS for 14-days. Treatment ended on day 16 and SGM3 mice were imaged on days 21 and 28. The slope of the line for mice treated with hMIL33B-H3 is 8771 (photons/(sec*day)) while in the control group the slope is −1043 (photons/(sec*day)). The results indicate that treatment with the hMIL33B-H3 antibody reduce the generation of RONS in the head and halting treatment of the antibody re-initiates production of RONS in the brain. Following the halt of treatment for 2-weeks (depicted in FIG. 9A), the mice were subject to a second round of treatment for another week. FIG. 9B provides a graph of the head RONS bursts following re-treatment with the hMIL33B-H3 antibody (triangles) and with PBS (circles). The slope of the line for mice treated with hMIL33B-H3 antibody is −15143 (photons/(sec*day)) while in the control group the slope is −749 (photons/(sec*day)). The depletion of head RONS bursts in the mice following re-treatment demonstrate the direct effect the hMIL-33B-H3 antibody has on reducing RONS in the brain. Taken collectively, these results demonstrate the role of the hMIL33B-H3 as an effective modulator of inflammation.

ADDITIONAL SEQUENCES

SEQ ID NO: 27: AHF15945 VH nucleotide sequence
ATGGGCTGGTCATGCATTATTCTGTTTCTGGTCGCAACTGCTACAGGCGTGCATAGTC
AAGTTCAGCTGGTGCAGAGCGGCGCCGAGGTGAAAAAGCCCGGCGCTAGCGTGAAA
GTGTCCTGCAAGGCCAGCGGATACACCTTCACCTCTTATGTGATGCACTGGGTGCGG
CAGGCCCCTGGCCAGCGGCTGGAATGGATGGGCTACATCAACAGCTACAGCGACGG
CACCAAGTACAACGAGAAGTTCAAGGGCAGAGTGACCATCACCTCCGATACAAGCG
CCTCTACCGCCTACATGGAACTGAGCAGCCTGAGAAGCGAGGACACAGCCGTGTACT
ACTGTGCCAGATGGGGAGGCCTGGGAAATGGCGCTATGGACTACTGGGGCCAGGGC
ACACTGGTCACCGTGTCTAGCGCCAGCACCAAGGGCCCTTCCGTGTTTCCACTGGCC
CCCTCCTCTAAATCCACATCTGGCGGCACCGCCGCCCTGGGCTGTCTGGTGAAGGAC
TACTTCCCAGAGCCTGTGACAGTGTCCTGGAACTCTGGCGCCCTGACATCCGGCGTG
CACACATTTCCAGCCGTGCTGCAGAGCTCCGGCCTGTACAGCCTGTCTAGCGTGGTG
ACAGTGCCCTCCTCTAGCCTGGGCACACAGACCTATATCTGCAACGTGAATCACAAG
CCAAGCAATACCAAGGTGGACAAGAAGGTGGAGCCCAAGTCCTGTGATAAGACACA
CACCTGCCCCCCTTGTCCTGCTCCCGAGCTGCTGGGCGGCCCTAGCGTGTTCCTGTTT
CCACCCAAGCCTAAGGACACCCTGATGATCTCCCGGACACCCGAGGTGACCTGCGTG
GTGGTGGACGTGTCTCACGAGGATCCTGAGGTGAAGTTCAACTGGTATGTGGATGGC
GTGGAGGTGCACAATGCCAAGACCAAGCCCAGAGAGGAGCAGTACAACTCTACATA
TAGGGTGGTGAGCGTGCTGACCGTGCTGCACCAGGACTGGCTGAACGGCAAGGAGT
ATAAGTGCAAGGTGTCCAATAAGGCCCTGCCCGCCCCCATCGAGAAGACAATCAGC
AAGGCCAAGGGCCAGCCTCGGGAGCCACAGGTGTACACCCTGCCTCCATCCAGAGA
CGAGCTGACAAAGAACCAGGTGTCTCTGACATGTCTGGTGAAGGGCTTCTATCCTAG
CGATATCGCCGTGGAGTGGGAGTCCAATGGCCAGCCAGAGAACAATTACAAGACCA
CACCCCCTGTGCTGGACTCCGATGGCTCCTTCTTTCTGTATTCCAAGCTGACCGTGGA
TAAGTCTCGGTGGCAGCAGGGCAACGTGTTCAGCTGTTCCGTGATGCACGAAGCCCT
GCATAATCACTATACTCAGAAATCCCTGTCCCTGTCACCTGGAAAGTGATAA
SEQ ID NO: 28: AHF15938 VH nucleotide sequence
ATGGGCTGGTCATGCATTATTCTGTTTCTGGTCGCAACTGCTACAGGCGTGCATAGTC
AAGTGCAGCTGGTCCAGAGCGGCGCCGAGGTGAAAAAGCCCGGCGCTTCTGTTAAA
GTGTCCTGCAAGGCCTCTGGATATACATTCACCTCCTACGTGATGCACTGGGTGCGG
CAGGCCCCTGGCCAGCGGCTGGAATGGATGGGCTACATCAACAGCTACAGCGACGG
CACAAAGTACAACGAGAAGTTCAAGGGCAGAGCCACAATCACCAGAGATACCAGCG
CCAGCACCGCCTACATGGAACTGAGCAGCCTGAGAAGCGAGGACACCGCCGTGTAC
TACTGTGCCAGATGGGGCGGACTGGGCAATGGCGCTATGGACTACTGGGGACAGGG
CACCCTGGTGACCGTGTCTAGCGCCAGCACCAAGGGCCCTTCCGTGTTTCCACTGGC
CCCCTCCTCTAAATCCACATCTGGCGGCACCGCCGCCCTGGGCTGTCTGGTGAAGGA
CTACTTCCCAGAGCCTGTGACAGTGTCCTGGAACTCTGGCGCCCTGACATCCGGCGT
GCACACATTTCCAGCCGTGCTGCAGAGCTCCGGCCTGTACAGCCTGTCTAGCGTGGT
GACAGTGCCCTCCTCTAGCCTGGGCACACAGACCTATATCTGCAACGTGAATCACAA
GCCAAGCAATACCAAGGTGGACAAGAAGGTGGAGCCCAAGTCCTGTGATAAGACAC
ACACCTGCCCCCCTTGTCCTGCTCCCGAGCTGCTGGGCGGCCCTAGCGTGTTCCTGTT
TCCACCCAAGCCTAAGGACACCCTGATGATCTCCCGGACACCCGAGGTGACCTGCGT
GGTGGTGGACGTGTCTCACGAGGATCCTGAGGTGAAGTTCAACTGGTATGTGGATGG
CGTGGAGGTGCACAATGCCAAGACCAAGCCCAGAGAGGAGCAGTACAACTCTACAT
ATAGGGTGGTGAGCGTGCTGACCGTGCTGCACCAGGACTGGCTGAACGGCAAGGAG
TATAAGTGCAAGGTGTCCAATAAGGCCCTGCCCGCCCCCATCGAGAAGACAATCAGC
AAGGCCAAGGGCCAGCCTCGGGAGCCACAGGTGTACACCCTGCCTCCATCCAGAGA
CGAGCTGACAAAGAACCAGGTGTCTCTGACATGTCTGGTGAAGGGCTTCTATCCTAG
CGATATCGCCGTGGAGTGGGAGTCCAATGGCCAGCCAGAGAACAATTACAAGACCA
CACCCCCTGTGCTGGACTCCGATGGCTCCTTCTTTCTGTATTCCAAGCTGACCGTGGA
TAAGTCTCGGTGGCAGCAGGGCAACGTGTTCAGCTGTTCCGTGATGCACGAAGCCCT
GCATAATCACTATACTCAGAAATCCCTGTCCCTGTCACCTGGAAAGTGATAA
SEQ ID NO: 29: AHF15948 VH nucleotide sequence
ATGGGCTGGTCATGCATTATTCTGTTTCTGGTCGCAACTGCTACAGGCGTGCATAGT
CAAGTGCAGCTGGTTCAGAGCGGCGCCGAGGTGAAAAAGCCCGGAGCTAGCGTGAA
AGTGTCCTGCAAGGCCTCTGGCTACACCTTCACCTCCTACGTGATGCACTGGGTCAG
ACAGGCCCCTGGCCAGCGGCTGGAATGGATGGGCTATATCAACAGCTACAGCGACG
GCACAAAGTACAACGAGAAGTTCAAGGGCAGAGTGACCATCACCAGAGATACCAG
CGCCTCTACAGCCTACATGGAACTGAGCAGCCTGCGGAGCGAGGACACCGCCGTGT
ACTACTGTGCCAGATGGGGAGGACTGGGCAATGGCGCTATGGACTACTGGGGCCAG
GGCACCCTGGTGACAGTGTCTAGCGCCAGCACCAAGGGCCCTTCCGTGTTTCCACTG
GCCCCCTCCTCTAAATCCACATCTGGCGGCACCGCCGCCCTGGGCTGTCTGGTGAAG
GACTACTTCCCAGAGCCTGTGACAGTGTCCTGGAACTCTGGCGCCCTGACATCCGGC
GTGCACACATTTCCAGCCGTGCTGCAGAGCTCCGGCCTGTACAGCCTGTCTAGCGTG
GTGACAGTGCCCTCCTCTAGCCTGGGCACACAGACCTATATCTGCAACGTGAATCAC
AAGCCAAGCAATACCAAGGTGGACAAGAAGGTGGAGCCCAAGTCCTGTGATAAGAC
ACACACCTGCCCCCCTTGTCCTGCTCCCGAGCTGCTGGGCGGCCCTAGCGTGTTCCT
GTTTCCACCCAAGCCTAAGGACACCCTGATGATCTCCCGGACACCCGAGGTGACCTG
CGTGGTGGTGGACGTGTCTCACGAGGATCCTGAGGTGAAGTTCAACTGGTATGTGGA
TGGCGTGGAGGTGCACAATGCCAAGACCAAGCCCAGAGAGGAGCAGTACAACTCTA
CATATAGGGTGGTGAGCGTGCTGACCGTGCTGCACCAGGACTGGCTGAACGGCAAG
GAGTATAAGTGCAAGGTGTCCAATAAGGCCCTGCCCGCCCCCATCGAGAAGACAAT
CAGCAAGGCCAAGGGCCAGCCTCGGGAGCCACAGGTGTACACCCTGCCTCCATCCA
GAGACGAGCTGACAAAGAACCAGGTGTCTCTGACATGTCTGGTGAAGGGCTTCTAT
CCTAGCGATATCGCCGTGGAGTGGGAGTCCAATGGCCAGCCAGAGAACAATTACAA
GACCACACCCCCTGTGCTGGACTCCGATGGCTTCCTTCTTTCTGTATTCCAAGCTGAC
CGTGGATAAGTCTCGGTGGCAGCAGGGCAACGTGTTCAGCTGTTCCGTGATGCACGA
AGCCCTGCATAATCACTATACTCAGAAATCCCTGTCCCTGTCACCTGGAAAGTGATA
A
SEQ ID NO: 30: AHF 15938 VL nucleotide sequence (same for AHF15945 and 15948)
ATGGGCTGGTCATGTATTATTCTGTTTCTGGTCGCAACTGCTACAGGGGTCCATAGT
GAGATCGTGCTGACACAGAGCCCTGCTACACTGAGCCTGAGCCCCGGCGAGCGGGC
CACACTCTCCTGCAGCGTGTCCAGCTCTGTCAACTACATGCACTGGTATCAGCAGAA
ACCTGGCCAGGCCCCTAGAAGACTGATCTACGACACCAGCAAGCTGGCCTCTGGAA
TCCCAGCCAGATTCAGCGGATCTGGCAGCGGCACCGATTACACCCTGACCATCAGC
AGCCTGGAACCTGAGGACTTCGCCGTGTACTACTGTCAGCAATGGACCTCCAACCCC
CTGACCTTTGGCCAGGGCACCAAGCTGGAAATCAAGAGGACAGTGGCCGCCCCAAG
CGTGTTCATCTTTCCCCCTTCCGACGAGCAGCTGAAGTCTGGCACCGCCAGCGTGGT
GTGCCTGCTGAACAACTTCTACCCTCGGGAGGCCAAGGTCCAGTGGAAGGTGGATA
ACGCCCTGCAGTCTGGCAATAGCCAGGAGTCCGTGACCGAGCAGGACTCTAAGGAT
AGCACATATTCCCTGTCTAGCACCCTGACACTGAGCAAGGCCGATTACGAGAAGCA
CAAGGTGTATGCCTGTGAAGTCACCCATCAGGGGCTGTCATCACCCGTCACTAAGTC
ATTCAATCGCGGAGAATGCTGATAA
SEQ ID NO: 31 U155QGF100-chimeric-VH amino acid sequence
MGWSCIILFLVATATGVHSEVQLQQSGPELVKPGASVKMSCKASGYTFTSYVMHWVRQ
SPGQGLEGIGYINSYSDGTKYNEKFKGKATLTSDKSSSTAYMELSGLTSEDSAVYYCAR
WGGLGNGAMDYWGQGTSVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
K
SEQ ID NO: 32 U1J155QGF100-grafted-VH amino acid sequence
MGWSCIILFLVATATGVHSQVQLVQSGAEVKKPGASVKVSCKASGYTFTSYVMHWVR
QAPGQRLEWMGYINSYSDGTKYNEKFKGRVTITRDTSASTAYMELSSLRSEDTAVYYC
ARWGGLGNGAMDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
K
SEQ ID NO: 33 U155QGF100-chimeric-VL amino acid sequence
MGWSCIILFLVATATGVHSQIVLTQSPAIMSASPGEKVTMTCSVSSSVNYMHWYQQKSG
TSPKRWIYDTSKLASGVPARFSASGSGTSYSLTISSMEAEDAATYYCQQWTSNPLTFGA
GTKLELKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
SEQ ID NO: 34 U155QGF100-grafted-VL amino acid sequence
MGWSCIILFLVATATGVHSEIVLTQSPATLSLSPGERATLSCSVSSSVNYMHWYQQKPG
QAPRLLIYDTSKLASGIPARFSGSGSGTDFTLTISSLEPEDFAVYYCQQWTSNPLTFGQGT
KLEIKRTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
SEQ ID NO: 35 FLAG
DYKDDDDK
SEQ ID NO: 36 polyhistidine (6-His)
HHHHHH
SEQ ID NO: 37 alternative His tag
HEHEHE
SEQ ID NO: 38 hemagglutinin (HA)
YPYDVPDYA
SEQ ID NO: 39: CD8α hinge
TTTPAPRPPTPAPTIASQPLSLRPEACRPAAGGAVHTRGLDFACD
SEQ ID NO: 40: CD8α transmembrane domain
IYIWAPLAGTCGVLLLSLVITLYC
SEQ ID NO: 41: 4-1BB signaling domain
KRGRKKLLYIFKQPFMRPVQTTQEEDGCSCRFPEEEEGGCEL
SEQ ID NO: 42: scFv
EIVLTQSPATLSLSPGERATLSCSVSSSVNYMHWYQQKPGQAPRRLIYDTSKLASGIPARFSGSGS
GTDYTLTISSLEPEDFAVYYCQQWTSNPLTFGQGTKLEIKGSGSGSGSGSGSGSGSQVQLVQSG
AEVKKPGASVKVSCKASGYTFTSYVMHWVRQAPGQRLEWMGYINSYSDGTKYNEKFKGRVTI
TRDTSASTAYMELSSLRSEDTAVYYCARWGGLGNGAMDYWGQGTLVTVSS
SEQ ID NO: 43: scFv linker
GGGGSGGGGSGGGGS
SEQ ID NO: 44: scFv linker
GSGSGSGSGSGSGSGS

1. An isolated antibody or antibody fragment, comprising:
a heavy chain variable region (VH) having at least 90% identity to SEQ ID NO: 3 and comprising a VHCDR1 amino acid sequence comprising SEQ ID NO: 9, 15, or 21, a VHCDR2 amino acid sequence comprising SEQ ID NO: 10, 16, or 22, and a VHCDR3 amino acid sequence comprising SEQ ID NO: 11, 17, or 23 and having a tryptophan at position 47, a methionine at position 48, a valine at position 68, and an arginine at position 72 of SEQ ID NO: 3; and
a light chain variable region (VL) having at least 90% identity to SEQ ID NO: 4 and comprising a VLCDR1 amino acid sequence comprising SEQ ID NO: 12, 18, or 24, a VLCDR2 amino acid sequence comprising SEQ ID NO: 13, 19 or 25, and a VLCDR3 amino acid sequence comprising SEQ ID NO: 14, 20, or 26 and having an arginine at position 45 and a tyrosine at position 70 of SEQ ID NO: 4.

2.-3. (canceled)

4. The isolated antibody or antibody fragment of claim 1, wherein said antibody or antibody fragment comprises a heavy chain variable sequence as set forth in SEQ ID NO: 3 and a light chain variable sequence as set forth in SEQ ID NO: 4.

5. The isolated antibody or antibody fragment of claim 1, wherein the antibody fragment is a monovalent scFv (single chain fragment variable) antibody, divalent scFv, Fab fragment, F(ab′)2 fragment, F(ab′)3 fragment, Fv fragment, or single chain antibody.

6.-7. (canceled)

8. The isolated antibody or antibody fragment of claim 1, wherein the antibody or antibody fragment has
a) increased binding affinity for a 4Ig isoform of B7-H3; and
b) at least 350-fold increased selectivity for the 4Ig isoform of B7-H3 as compared to a 2Ig isoform.

9. The isolated antibody or antibody fragment of claim 1, wherein the antibody is conjugated or fused to an imaging agent, a cytotoxic agent, a metal, or a radioactive moiety.

10.-14. (canceled)

15. An isolated nucleic acid encoding the antibody heavy chain variable region and/or light chain variable region of the antibody or antibody fragment of claim 1.

16. (canceled)

17. A hybridoma or engineered cell comprising a nucleic acid encoding the antibody or antibody fragment of claim 1.

18. (canceled)

19. A method of making an isolated antibody or antibody fragment, comprising culturing the hybridoma or engineered cell of claim 17 under conditions that allow expression of the antibody and, optionally, isolating the antibody from the culture.

20. A chimeric antigen receptor (CAR) protein comprising an antigen binding domain comprising a heavy chain variable region (VH) having at least 90% identity to SEQ ID NO: 1, SEQ ID NO: 2, or SEQ ID NO: 3 and comprising a VHCDR1 amino acid sequence comprising SEQ ID NO: 9, 15, or 21, a VHCDR2 amino acid sequence comprising SEQ ID NO: 10, 16, or 22, and a VHCDR3 amino acid sequence comprising SEQ ID NO: 11, 17, or 23; and
a light chain variable region (VL) having at least 90% identity to SEQ ID NO: 4 and comprising a VLCDR1 amino acid sequence comprising SEQ ID NO: 12, 18, or 24, a VLCDR2 amino acid sequence comprising SEQ ID NO: 13, 19 or 25, and a VLCDR3 amino acid sequence comprising SEQ ID NO: 14, 20, or 26 and comprising an arginine corresponding to position 45 and a tyrosine corresponding to position 70 of SEQ ID NO: 4.

21-23. (canceled)

24. The CAR of claim 20, wherein the antigen binding domain comprises a heavy chain variable sequence having a sequence set forth in SEQ ID NO: 3 and a light chain variable sequence having a sequence set forth in SEQ ID NO: 4.

25.-30. (canceled)

31. A nucleic acid molecule encoding the CAR of claim 20.

32-33. (canceled)

34. An engineered cell comprising a nucleic acid molecule encoding the chimeric antigen receptor (CAR) of claim 20.

35-38. (canceled)

39. A pharmaceutical composition comprising a population of cells in accordance with claim 34 and a pharmaceutically acceptable carrier.

40. A method of treating cancer or an inflammatory condition in a human patient in need thereof comprising administering to the patient an anti-tumor effective amount of the pharmaceutical composition of claim 39.

41-49. (canceled)

50. A pharmaceutical composition comprising the isolated antibody or antibody fragment of claim 1 and a pharmaceutically acceptable carrier.

51. A method of treating cancer or an inflammatory condition in a human patient in need thereof comprising administering to the patient an anti-tumor effective amount of the pharmaceutical composition of claim 50.

52. The method of claim 51, wherein the patient has a cancer that has been determined to express an elevated level of B7-H3 relative to a healthy tissue.

53. The method of claim 51, wherein the patient has a cancer selected from a group consisting of a renal cancer, a pancreatic cancer, a colorectal cancer, a non-small cell lung cancer, an ovarian cancer, a bladder cancer, a melanoma, a prostate cancer, a breast cancer, a glioma, a lymphoma, and a neuroectodermal cancer.

54-57. (canceled)

58. A method for diagnosing a patient having cancer, comprising:
detecting, and optionally quantitating, an expression of B7-H3 in the cancer using an isolated antibody or antigen binding fragment of claim 1.

59-68. (canceled)

69. The method of claim 51, wherein the inflammatory condition is selected from the group consisting of systemic lupus erythematosus (SLE), Sjogren's syndrome, dermatitis, Type 1 diabetes, Type 2 diabetes, thyroiditis, Addison disease, pernicious anemia, autoimmune hepatitis, inflammatory bowel disease, multiple sclerosis, encephalitis, rheumatoid arthritis, myasthenia gravis, neuritis, primary biliary cholangitis, Goodpasture's disease, primary membranous nephropathy, cystitis, ovarian insufficiency, autoimmune orchitis, chronic obstructive pulmonary disease (COPD), asthma, pneumonitis, high blood pressure, heart disease, myositis, myocarditis, inflammatory arteritis (Takayasu arteritis, giant cell arteritis), lymphangitis, Parkinson's disease, or graft versus host disease.

70.-72. (canceled)
